SEQUENCE LISTING (74 sequences)

SEQ ID NO: 1 - protein, 9 aa, synthetic construct
VVGAVGVGK

SEQ ID NO: 2 - protein, 10 aa, synthetic construct
VVVGAVGVGK

SEQ ID NO: 3 - protein, 6 aa, synthetic construct
TSGFNG

SEQ ID NO: 4 - protein, 6 aa, synthetic construct
NSMFDY

SEQ ID NO: 5 - protein, 6 aa, synthetic construct
NVLDGL

SEQ ID NO: 6 - protein, 7 aa, synthetic construct
ISSIKDK

SEQ ID NO: 7 - protein, 12 aa, synthetic construct
AVKGRNSGYALN

SEQ ID NO: 8 - protein, 12 aa, synthetic construct
AASERGRDDKII

SEQ ID NO: 9 - protein, 5 aa, synthetic construct
SGDLS

SEQ ID NO: 10 - protein, 5 aa, synthetic construct
SEHNR

SEQ ID NO: 11 - protein, 6 aa, synthetic construct
YYNGEE

SEQ ID NO: 12 - protein, 6 aa, synthetic construct
FQNEAQ

SEQ ID NO: 13 - protein, 13 aa, synthetic construct
ASTIQGAAQETQY

SEQ ID NO: 14 - protein, 12 aa, synthetic construct
ASSFSRGPETQY

SEQ ID NO: 15 - protein, 251 aa, synthetic construct
QNIDQPTEMTATEGAIVQINCTYQTSGFNGLFWYQQHAGEAPTFLSYNVLDGLEEKGRFSSFLSRSKGYSYLLLKELQMKDSASYLCAVKGRNSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 259 aa, synthetic construct
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASERGRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 291 aa, synthetic construct
GVTQTPKHLITATGQRVTLRCSPRSGDLSVYWYQQSLDQGLQFLIQYYNGEERAKGNILERFSAQQFPDLHSELNLSSLELGDSALYFCASTIQGAAQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 18 - protein, 291 aa, synthetic construct
GVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSRGPETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - DNA, 18 bp, synthetic construct
  source 1..18: mol_type = other DNA
acatctgggttcaacggg

SEQ ID NO: 20 - DNA, 18 bp, synthetic construct
  source 1..18: mol_type = other DNA
aacagcatgtttgattat

SEQ ID NO: 21 - DNA, 18 bp, synthetic construct
  source 1..18: mol_type = other DNA
aatgttctggatggtttg

SEQ ID NO: 22 - DNA, 21 bp, synthetic construct
  source 1..21: mol_type = other DNA
ataagttccattaaggataaa

SEQ ID NO: 23 - DNA, 36 bp, synthetic construct
  source 1..36: mol_type = other DNA
gctgtgaagggtaggaattccgggtatgcactcaac

SEQ ID NO: 24 - DNA, 36 bp, synthetic construct
  source 1..36: mol_type = other DNA
gcagcaagcgagcgaggcagagatgacaagatcatc

SEQ ID NO: 25 - DNA, 15 bp, synthetic construct
  source 1..15: mol_type = other DNA
tctggagacctctct

SEQ ID NO: 26 - DNA, 15 bp, synthetic construct
  source 1..15: mol_type = other DNA
tctgaacacaaccgc

SEQ ID NO: 27 - DNA, 18 bp, synthetic construct
  source 1..18: mol_type = other DNA
tattataatggagaagag

SEQ ID NO: 28 - DNA, 18 bp, synthetic construct
  source 1..18: mol_type = other DNA
ttccagaatgaagctcaa

SEQ ID NO: 29 - DNA, 39 bp, synthetic construct
  source 1..39: mol_type = other DNA
gccagcactatacagggggcggctcaagagacccagtac

SEQ ID NO: 30 - DNA, 36 bp, synthetic construct
  source 1..36: mol_type = other DNA
gccagcagcttttccagggggccagagacccagtac

SEQ ID NO: 31 - protein, 111 aa, synthetic construct
QNIDQPTEMTATEGAIVQINCTYQTSGFNGLFWYQQHAGEAPTFLSYNVLDGLEEKGRFSSFLSRSKGYSYLLLKELQMKDSASYLCAVKGRNSGYALNFGKGTSLLVTPH

SEQ ID NO: 32 - protein, 119 aa, synthetic construct
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASERGRDDKIIFGKGTRLHILPN

SEQ ID NO: 33 - DNA, 333 bp, synthetic construct
  source 1..333: mol_type = other DNA
caaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgaagggtaggaattccgggtatgcactcaacttcggcaaaggcacctcgctgttggtcacaccccat

SEQ ID NO: 34 - DNA, 357 bp, synthetic construct
  source 1..357: mol_type = other DNA
caacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgagcgaggcagagatgacaagatcatctttggaaaagggacacgacttcatattctccccaat

SEQ ID NO: 35 - protein, 113 aa, synthetic construct
GVTQTPKHLITATGQRVTLRCSPRSGDLSVYWYQQSLDQGLQFLIQYYNGEERAKGNILERFSAQQFPDLHSELNLSSLELGDSALYFCASTIQGAAQETQYFGPGTRLLVLE

SEQ ID NO: 36 - protein, 113 aa, synthetic construct
GVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSRGPETQYFGPGTRLLVLE

SEQ ID NO: 37 - DNA, 339 bp, synthetic construct
  source 1..339: mol_type = other DNA
ggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcactatacagggggcggctcaagagacccagtacttcgggccaggcacgcggctcctggtgctcgag

SEQ ID NO: 38 - DNA, 339 bp, synthetic construct
  source 1..339: mol_type = other DNA
ggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttccagggggccagagacccagtacttcgggccaggcacgcggctcctggtgctcgag

SEQ ID NO: 39 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 140 aa, synthetic construct; identical to SEQ ID NO: 39

SEQ ID NO: 41 - DNA, 423 bp, synthetic construct
  source 1..423: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 42 - DNA, 423 bp, synthetic construct; identical to SEQ ID NO: 41
  source 1..423: mol_type = other DNA

SEQ ID NO: 43 - protein, 178 aa, synthetic construct
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 44 - protein, 178 aa, synthetic construct; identical to SEQ ID NO: 43

SEQ ID NO: 45 - DNA, 534 bp, synthetic construct
  source 1..534: mol_type = other DNA
gacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 46 - DNA, 534 bp, synthetic construct; identical to SEQ ID NO: 45
  source 1..534: mol_type = other DNA

SEQ ID NO: 47 - protein, 27 aa, synthetic construct
RAKRSGSGATNFSLLKQAGDVEENPGP

SEQ ID NO: 48 - protein, 27 aa, synthetic construct
RAKRSGSGATNFSLLKQAGDVEENPGP

SEQ ID NO: 49 - DNA, 81 bp, synthetic construct
  source 1..81: mol_type = other DNA
cgtgccaagcgaagcggatctggcgccacgaacttctctctgttaaagcaagcaggagatgttgaagaaaaccccgggcct

SEQ ID NO: 50 - DNA, 81 bp, synthetic construct; identical to SEQ ID NO: 49
  source 1..81: mol_type = other DNA

SEQ ID NO: 51 - protein, 18 aa, synthetic construct
MWGVFLLYVSMKMGGTTG

SEQ ID NO: 52 - protein, 21 aa, synthetic construct
MAMLLGASVLILWLQPDWVNS

SEQ ID NO: 53 - DNA, 54 bp, synthetic construct
  source 1..54: mol_type = other DNA
atgtggggagttttccttctttatgtttccatgaagatgggaggcactacagga

SEQ ID NO: 54 - DNA, 63 bp, synthetic construct
  source 1..63: mol_type = other DNA
atggccatgctcctgggggcatcagtgctgattctgtggcttcagccagactgggtaaacagt

SEQ ID NO: 55 - protein, 21 aa, synthetic construct
MGFRLLCCVAFCLLGAGPVDS

SEQ ID NO: 56 - protein, 21 aa, synthetic construct
MGTSLLCWMALCLLGADHADT

SEQ ID NO: 57 - DNA, 63 bp, synthetic construct
  source 1..63: mol_type = other DNA
atgggcttcaggctcctctgctgtgtggccttttgtctcctgggagcaggcccagtggattct

SEQ ID NO: 58 - DNA, 63 bp, synthetic construct
  source 1..63: mol_type = other DNA
atgggcaccagcctcctctgctggatggccctgtgtctcctgggggcagatcacgcagatact

SEQ ID NO: 59 - protein, 608 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASTIQGAAQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVKGRNSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 619 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSRGPETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASERGRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - DNA, 1827 bp, synthetic construct
  source 1..1827: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcactatacagggggcggctcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggccgtgccaagcgaagcggatctggcgccacgaacttctctctgttaaagcaagcaggagatgttgaagaaaaccccgggcctatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgaagggtaggaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 62 - DNA, 1860 bp, synthetic construct
  source 1..1860: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttccagggggccagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggccgtgccaagcgaagcggatctggcgccacgaacttctctctgttaaagcaagcaggagatgttgaagaaaaccccgggcctatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgagcgaggcagagatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 63 - protein, 269 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVKGRNSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 280 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASERGRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - DNA, 810 bp, synthetic construct
  source 1..810: mol_type = other DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgaagggtaggaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 66 - DNA, 843 bp, synthetic construct
  source 1..843: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgagcgaggcagagatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 67 - protein, 312 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASTIQGAAQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 68 - protein, 312 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSRGPETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - DNA, 936 bp, synthetic construct
  source 1..936: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcactatacagggggcggctcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 70 - DNA, 936 bp, synthetic construct
  source 1..936: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttccagggggccagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 71 - DNA, 756 bp, synthetic construct
  source 1..756: mol_type = other DNA
caaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgaagggtaggaattccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 72 - DNA, 780 bp, synthetic construct
  source 1..780: mol_type = other DNA
caacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgagcgaggcagagatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 73 - DNA, 873 bp, synthetic construct
  source 1..873: mol_type = other DNA
ggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcactatacagggggcggctcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 74 - DNA, 873 bp, synthetic construct
  source 1..873: mol_type = other DNA
ggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttccagggggccagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

(19) United States
(12) Patent Application Publication
(10) Pub. No.: US 2026/0216333 A1
(43) Pub. Date: Jul. 30, 2026

(54) T CELL RECEPTORS TARGETING THE HIGHLY PREVALENT KRAS G12V MUTATION ON HLA-A*03:01 IN LUNG CANCER

(71) Applicant: BOARD OF REGENTS, THE UNIVERSITY OF TEXAS SYSTEM, Austin, TX (US)

(72) Inventors: Alexandre REUBEN, Houston, TX (US); Minying ZHANG, Houston, TX (US); John Victor HEYMACH, Houston, TX (US); Gregory A. LIZEE, Houston, TX (US); Peixin JIANG, Houston, TX (US)

(73) Assignee: BOARD OF REGENTS, THE UNIVERSITY OF TEXAS SYSTEM, Austin, TX (US)

(21) Appl. No.: 19/145,818

(22) PCT Filed: Jan. 4, 2024
(86) PCT No.: PCT/US2024/010344

§ 371 Date: Jul. 3, 2025

Related U.S. Application Data
(60) Provisional application No. 63/478,490, filed on Jan. 4, 2023.

(51) Int. Cl. A61K 40/32; A61K 38/00; A61K 40/11; A61K 40/42; A61P 35/00; C07K 14/705; C07K 16/40; C12N 5/0783

(52) U.S. Cl. CPC A61K 40/32; A61K 40/11; A61K 40/4244; A61P 35/00; C07K 14/7051; C07K 16/40; C12N 5/0636; A61K 38/00; C07K 2317/24; C07K 2317/31; C12N 2510/00

(57) ABSTRACT

The present disclosure is directed to recombinant T-cell Receptors (TCRs) that binds Kirsten Rat Sarcoma Viral Oncogene Homolog (KRAS) protein comprising a G12V mutation presented on a human leucocyte antigen-A (HLA-A)*03:01, polynucleotides encoding such TCRs, engineered T-cells comprising such TCRs, and methods of using the same.

### CROSS REFERENCE TO RELATED APPLICATIONS

The present application claims the priority benefit of U.S. Provisional Application No. 63/478,490, filed Jan. 4, 2023, which is hereby incorporated by reference in its entirety.

### REFERENCE TO SEQUENCE LISTING SUBMITTED ELECTRONICALLY

The content of the electronically submitted sequence listing (Name: 4443_029PC01_Seqlisting_ST26.xml; Size: 84,094 bytes; and Date of Creation: Nov. 16, 2023), filed with the application, is incorporated herein by reference in its entirety.

### FIELD OF THE DISCLOSURE

The present disclosure pertains to the medical field, including oncology and immunotherapy therapy. For example, the present disclosure provides T-cell Receptors (TCRs), T-cells, and T-cell therapies comprising the same, and methods of using the same.

### BACKGROUND

Lung cancer is a common form of cancer in men and women, and it is responsible for the highest number of cancer-related deaths globally. Because of the high rate (>50%) of late diagnosis, the 5 year overall survival rate of patients with lung cancer has improved very little over the past 3 decades, hovering around the 13%-16% range.

In recent years, therapies based on the activation of the patient's immune system (e.g., immunotherapy) have been used to treat cancer. In general, cancer immunotherapy aims to elicit and/or magnify the cytotoxic activity of tumor-cell-targeting immune cells, overcome immunosuppression in the tumor tissues, and boost the host immune system to fight against cancer. Adoptive cell transfer (ACT) therapy is a type of immunotherapy in which immune cells that target human leukocyte antigen (HLA)-antigen complexes are isolated from a patient, amplified or engineered in vitro, and subsequently infused back into the patient to achieve precise cytotoxicity on tumor cells expressing the targeting antigens.

Neoantigens (a class of tumor-specific antigens) have been targeted for inducing an immune response in cancer patients. Cytolytic T lymphocytes (CTLs) that recognize mutated peptides (e.g., neoantigens) presented at the tumor cell surface by HLA class I molecules represent antitumor immune cells that are capable of causing regression of large tumors in cancer patients. T-cell receptors (TCRs) derived from such tumor antigen-specific T cells can be cloned and isolated to create engineered TCR-T cells.

However, as a natural consequence of high HLA diversity and the individual nature of most tumor-associated mutations, very few neoantigen targets are shared between patients, hampering the use of engineered neoantigen-targeting TCR-T cells as therapeutic agents.

### BRIEF SUMMARY

Certain aspects of the disclosure are directed to a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the disclosure is directed to recombinant polynucleotides encoding such TCRs, and to vectors comprising such recombinant polynucleotides encoding such TCRs. In some aspects, the (HLA-A)*03:01 allele presents an epitope comprising the amino acid sequence of VVGAVGVGK (SEQ ID NO: 1). In some aspects, the (HLA-A)*03:01 allele presents an epitope comprising the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

Certain aspects of the disclosure are directed to modified cells (e.g., engineered T cells) expressing a TCR, or an antigen-binding fragment thereof, which is capable of binding to an epitope of a KRAS protein comprising a G12V mutation presented on a HLA-A*03:01 allele, and/or capable of recognition of a cancer cell expressing a KRAS protein comprising a G12V mutation, which comprises an epitope presented on a human leucocyte antigen-A (HLA-A)*03:01 allele. In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

Certain aspects of the disclosure are directed to methods for prophylaxis and/or therapy of a subject diagnosed with, suspected of having or at risk for developing or recurrence of a cancer (e.g., a lung cancer), comprising administering to the subject a TCR or antigen-binding fragment thereof disclosed herein; a polynucleotide encoding such a TCR or antigen-binding fragment thereof, a vector comprising a polynucleotide encoding such a TCR or antigen-binding fragment thereof, or a modified cell (e.g., an engineered T cell) expressing such a TCR or antigen-binding fragment thereof on the cell surface.

In some aspects, the T-cell Receptor (TCR), or antigen-binding fragment thereof, comprises: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO:17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68, wherein the TCR binds an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the TCR specifically binds to the epitope. In some aspects, the TCR is a recombinant TCR. In some aspects, the TCR alpha chain comprises CDR1α, CDR2α, and CDR3α of SEQ ID NO: 15, 31 or 63. In some aspects, the TCR alpha chain comprises CDR1α, CDR2α, and CDR3α of SEQ ID NO: 16, 32, or 64. In some aspects, the TCR beta chain comprises CDR1β, CDR2β, and CDR3β of SEQ ID NO: 17, 35, or 67. In some aspects, the TCR beta chain comprises CDR1β, CDR2β, and CDR3β of SEQ ID NO: 18, 36, or 68.

In some aspects, the T cell receptor (TCR), or antigen-binding fragment thereof, of the disclosure comprises: (i) a variable TCR alpha chain complementarity determining region 1 (CDR1α) comprising the amino acid sequence of SEQ ID NO: 3, or 4 (ii) a variable TCR alpha chain complementarity determining region 2 (CDR2α) comprising the amino acid sequence of SEQ ID NO: 5, or 6, and (iii) a variable TCR alpha chain complementarity determining region 3 (CDR3α) comprising the amino acid sequence of SEQ ID NO: 7, or 8, (iv) a variable TCR beta chain complementarity determining region 1 (CDR1β) comprising the amino acid sequence of SEQ ID NO: 9, or 10, (v) a variable TCR beta chain complementarity determining region 2 (CDR2β) comprising the amino acid sequence of SEQ ID NO: 11, or 12, and (vi) a variable TCR beta chain complementarity determining region 3 (CDR3β) comprising the amino acid sequence of SEQ ID NO: 13, or 14. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the T cell receptor (TCR), or antigen-binding fragment thereof, of the disclosure comprises (a) an alpha chain (TCR-α) comprising a CDR1α having the sequence of SEQ ID NO: 3, or 4, a CDR2α having the sequence of SEQ ID NO: 5, or 6, a CDR3α having the sequence of SEQ ID NO: 7, or 8; and (b) a beta chain (TCR-β) comprising a CDR1β having the sequence of SEQ ID NO: 9, or 10, a CDR2β having the sequence of SEQ ID NO: 11, or 12, and a CDR3β having the sequence of SEQ ID NO: 13, or 14. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the T cell receptor (TCR), or antigen-binding fragment thereof of the disclosure comprises an alpha chain (TCR-α) and a beta chain (TCR-β), wherein the TCR-α comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64, and the TCR-β comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68. In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31 or 63. In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 17, 35, or 67. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 18, 36, or 68.

In some aspects, the T cell receptor (TCR), or antigen-binding fragment thereof, of the disclosure comprises (a) an alpha chain (TCR-α) comprising an amino acid sequence of SEQ ID NO: 15, 16, 31, 32, 63, or 64, and (b) a beta chain (TCR-β) comprising an amino acid sequence of SEQ ID NO: 17, 18, 35, 36, 67, or 68. In some aspects, the TCR alpha chain comprises an amino acid sequence of SEQ ID NO: 15, 31 or 63. In some aspects, the TCR alpha chain comprises an amino acid sequence of SEQ ID NO: 16, 32, or 64. In some aspects, the TCR beta chain comprises an amino acid sequence of SEQ ID NO: 17, 35, or 67. In some aspects, the TCR beta chain comprises an amino acid sequence of SEQ ID NO: 18, 36, or 68.

In some aspects, the T cell receptor (TCR), or antigen-binding fragment thereof, of the disclosure is humanized or chimeric.

In some aspects, the disclosure is directed to a polynucleotide encoding the TCR, or antigen-binding fragment thereof, of the disclosure. In some aspects, the polynucleotide encodes the alpha chain (TCR-α) of the recombinant TCR, or antigen-binding fragment thereof, of the disclosure. In some aspects, the polynucleotide encodes the beta chain (TCR-β) of the recombinant TCR, or an antigen-binding fragment thereof, of the disclosure. In some aspects, the polynucleotide encodes the alpha chain (TCR-α) and the beta chain (TCR-β) of the recombinant TCR, or antigen-binding fragments thereof, of the disclosure.

In some aspects, the disclosure is directed to an expression vector comprising the polynucleotide encoding the TCR, or antigen-binding fragment thereof, of the disclosure. In some aspects, the vector comprises a polynucleotide encoding the alpha chain (TCR-α) of the recombinant TCR, or antigen-binding fragment thereof, of the disclosure. In some aspects, the vector comprises a polynucleotide encoding the beta chain (TCR-β) of the recombinant TCR, or antigen-binding fragment thereof, of the disclosure. In some aspects, the vector comprises a polynucleotide encoding the alpha chain (TCR-α) and the beta chain (TCR-β) of the recombinant TCR, or antigen-binding fragment thereof, of the disclosure. In some aspects, the expression vector comprises a promoter.

In some aspects, the disclosure is directed to a host cell comprising the T cell receptor (TCR), or antigen-binding fragment thereof, the polynucleotide, or the expression vector, disclosed herein. In some aspects, the host cell comprises a T cell. In some aspects, the host cell comprises a CD4+ cell. In some aspects, the host cell comprises a CD8+ cell. In some aspects, the host cell comprises a CD4+/CD8+ cell.

In some aspects, the disclosure is directed to a T cell comprising the T cell receptor (TCR), or antigen-binding fragment thereof, a polynucleotide, or an expression vector, disclosed herein. In some aspects, the T cell is a CD4+ cell. In some aspects, the T cell is a CD8+ cell. In some aspects, the T cell is a CD4+/CD8+ cell.

In some aspects, the disclosure is directed to a modified T cell comprising the T cell receptor (TCR), or antigen-binding fragment thereof, the polynucleotide, or the expression vector, disclosed herein. In some aspects, the T cell receptor (TCR) is expressed on the surface of the modified T cell.

In some aspects, the modified T cell is capable of recognition of a cancer cell expressing a Kirsten Rat Sarcoma Viral Oncogene Homolog (KRAS) protein comprising a G12V mutation, which comprises an epitope presented on the human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the recognition of the cancer cell comprises a human leucocyte antigen-A (HLA-A)*03:01-restricted binding of the TCR to the epitope of the Kirsten Rat Sarcoma Viral Oncogene Homolog (KRAS) protein comprising the G12V mutation expressed by the cancer cell. In some aspects, the recombinant TCR is humanized or chimeric.

In some aspects, the disclosure is directed to a Bi-specific T-cell engager (BiTE) comprising a TCR, or antigen-binding fragment thereof, of the disclosure.

In some aspects, the disclosure is directed to a modified T cell comprising a T cell receptor (TCR) comprising: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68, wherein the TCR binds an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the TCR is expressed on the surface of the modified T cell. In some aspects, the modified T cell comprises a TCR alpha chain comprising CDR1α, CDR2α, and CDR3α of SEQ ID NO: 15, 31 or 63. In some aspects, the modified T cell comprises a TCR alpha chain comprising CDR1α, CDR2α, and CDR3α of SEQ ID NO: 16, 32, or 64. In some aspects, the modified T cell comprises a TCR beta chain comprising CDR1β, CDR2β, and CDR3β of SEQ ID NO: 17, 35, or 67. In some aspects, the modified T cell comprises a TCR beta chain comprising CDR1β, CDR2β, and CDR3β of SEQ ID NO: 18, 36, or 68.

In some aspects, the modified T cell is capable of recognition of a cancer cell expressing the Kirsten Rat Sarcoma Viral Oncogene Homolog (KRAS) protein comprising the G12V mutation, which comprises the epitope presented on the human leucocyte antigen-A (HLA-A)*03:01. In some aspects, the recognition of the cancer cell comprises a human leucocyte antigen-A (HLA-A)*03:01-restricted binding of the TCR to the epitope of the Kirsten Rat Sarcoma Viral Oncogene Homolog (KRAS) protein comprising the G12V mutation expressed by the cancer cell. In some aspects, the TCR is humanized or chimeric.

In some aspects, the disclosure is directed to a bi-specific T-cell engager (BiTE) comprising a T cell receptor (TCR) comprising: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68, wherein the TCR binds an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the BiTE comprises a TCR alpha chain comprising CDR1α, CDR2α, and CDR3α of SEQ ID NO: 15, 31 or 63. In some aspects, the BiTE comprises a TCR alpha chain comprising CDR1α, CDR2α, and CDR3α of SEQ ID NO: 16, 32, or 64. In some aspects, the BiTE comprises a TCR beta chain comprising CDR1β, CDR2β, and CDR3β of SEQ ID NO: 17, 35, or 67. In some aspects, the BiTE comprises a TCR beta chain comprising CDR1β, CDR2β, and CDR3β of SEQ ID NO: 18, 36, or 68.

In some aspects, the modified T cell or the BiTE of the disclosure, comprise (i) a CDR1α comprising the sequence of SEQ ID NO: 3, or 4, (ii) a CDR2α comprising the sequence of SEQ ID NO: 5, or 6, (iii) a CDR3α comprising the sequence of SEQ ID NO: 7, or 8, (iv) a CDR1β comprising the sequence of SEQ ID NO: 9, or 10, (v) a CDR2β comprising the sequence of SEQ ID NO: 11, or 12, and (vi) a CDR3β comprising the sequence of SEQ ID NO: 13, or 14. In some aspects, the modified T cell or the BiTE comprises CDR1α, CDR2α, and CDR3α comprising or consisting of SEQ ID NOs: 3, 5, and 7, respectively. In some aspects, the modified T cell or the BiTE comprises CDR1α, CDR2α, and CDR3α comprising or consisting of SEQ ID NOs: 4, 6, and 8, respectively. In some aspects, the modified T cell or the BiTE comprises CDR1β, CDR2β, and CDR3β comprising or consisting of SEQ ID NOs: 9, 11, and 13, respectively. In some aspects, the modified T cell or the BiTE comprises CDR1β, CDR2β, and CDR3β comprising or consisting of SEQ ID NOs: 10, 12, and 14, respectively.

In some aspects, the modified T cell or the BiTE of the disclosure, comprise a TCR alpha chain comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64; and a TCR beta chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68. In some aspects, the TCR alpha chain comprises an amino acid sequence of SEQ ID NO: 15, 16, 31, 32, 63, or 64, and the TCR beta chain comprises an amino acid sequence of SEQ ID NO: 17, 18, 35, 36, 67, or 68. In some aspects, the modified T cell or the BiTE comprises a TCR alpha chain comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31 or 63. In some aspects, the modified T cell or the BiTE comprises a TCR alpha chain comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64. In some aspects, the modified T cell or the BiTE comprises a TCR beta chain comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 17, 35, or 67. In some aspects, the modified T cell or the BiTE comprises a TCR beta chain comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 18, 36, or 68.

In some aspects, the disclosure is directed to a method for prophylaxis and/or therapy of a subject diagnosed with, suspected of having or at risk for developing or recurrence of a cancer (e.g., lung cancer), comprising administering to the subject the recombinant TCR, the polynucleotide, the vector, the host cell, the T cell, the modified T cell, or the BiTE of the disclosure. In some aspects, the cancer comprises cancer cells expressing a Kirsten Rat Sarcoma Viral Oncogene Homolog (KRAS) protein comprising a G12V mutation, which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the cancer is selected from the group consisting of: a lung cancer (e.g., a non-small cell lung cancer (NSCLC)), a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, and any combination thereof. In some aspects, the cancer is selected from the group consisting of: a lung cancer (e.g., a non-small cell lung cancer (NSCLC)), a colorectal cancer, a pancreatic cancer, and any combination thereof. In some aspects, the cancer is a non-small cell lung cancer (NSCLC). In some aspects, the cancer is a tumor, e.g., a primary tumor or metastatic tumor.

In some aspects, the subject is a human.

In some aspects, method for prophylaxis and/or therapy of the disclosure comprises removing a T cell from the subject, and modifying (e.g., engineering) the T cell by introducing into the T cell a recombinant TCR, or antigen-binding fragment thereof, a polynucleotide, or a vector of the disclosure prior to the administering to the subject.

In some aspects, the subject is a human.

### BRIEF DESCRIPTION OF THE DRAWINGS

FIG. 1 shows flow cytometry analysis indicating the proportion of Kirsten Rat Sarcoma Viral Oncogene Homolog (KRAS) protein comprising a G12V mutation presented on a human leucocyte antigen-A (HLA-A)*03:01 antigen-specific T cells from healthy donor blood following a DC stimulation.

FIG. 2 shows flow cytometry analysis indicating the proportion of Kirsten Rat Sarcoma Viral Oncogene Homolog (KRAS) protein comprising a G12V mutation presented on a human leucocyte antigen-A (HLA-A)*03:01 antigen-specific T cells from healthy donor blood following the first rapid expansion protocol.

FIG. 3 shows flow cytometry analysis indicating the proportion of Kirsten Rat Sarcoma Viral Oncogene Homolog (KRAS) protein comprising a G12V mutation presented on a human leucocyte antigen-A (HLA-A)*03:01 antigen-specific T cells from healthy donor blood following the second rapid expansion protocol, and used in killing and TCR α/β determination.

FIG. 4A-B show a flow cytometry analysis scatter plot that indicates the recombinant TCRs (TCR-1 and TCR-2) can be expressed in normal PBMC following TCR transduction and rapid expansion.

FIG. 5A-B show the ability of both TCR-1 and TCR-2 transduced T cell populations to kill targeted tumor cells expressing the antigen of interest endogenously by chromium-51 release assay.

### DETAILED DESCRIPTION OF THE DISCLOSURE

### Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA (e.g., messenger RNA (mRNA) or plasmid DNA (pDNA)). The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides. By “isolated” nucleic acid or polynucleotide is intended a nucleic acid molecule, DNA or RNA, which has been removed from its native environment. An isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotides or a nucleic acid can be or can include a regulatory element such as a promoter, ribosome binding site, or a transcription terminator. Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

As used herein, the term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell. In some aspects, the TCR or the T-cell of the disclosure is isolated.

As used herein, the term “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus).

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

As used herein, the term “transfer vector”, or “delivery vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. Therefore, a transfer vector can include autonomously replicating plasmids or viruses. The term transfer vector should also be interpreted to further include non-plasmid and non-viral compounds that facilitate the transfer of nucleic acids into cells, such as polylysine compounds, liposomes, and the like. Examples of virus transfer vectors include, but are not limited to, adenoviral vectors, adeno-associated virus vectors, retroviral vectors, lentiviral vectors, and the like.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity. An alignment for purposes of determining percent nucleic acid sequence identity can be carried out according to procedures known in the art, as described for example in EP 1 241 179 B1. For example, a skilled person can use publicly available computer software, such as using publicly available computer software such as BLAST, BLAST-2, ALIGN or Megalign (DNASTAR) software. A skilled person can determine appropriate parameters for measuring alignment, including any algorithms needed to achieve maximal alignment over the full length of the sequences subjected to comparison. According to a preferred embodiment, the % identity values can be generated using the WU-BLAST-2 computer program. According to a preferred embodiment, the following computer program and parameters are used: The identity values used herein are generated by the BLASTN module of WU-BLAST-2 set to the default parameters, with overlap span and overlap fraction set to 1 and 0.125, respectively. A % nucleic acid sequence identity value can be obtained by dividing (a) the number of matching identical nucleotides between a particular nucleic acid sequence as set forth herein which is subjected to comparison (e.g. a particular nucleic acid sequence characterized by a sequence identifier in the sequence listings), and the comparison nucleic acid molecule of interest to be compared, for example the number of matching identical nucleotides as determined by WU-BLAST-2, by (b) the total number of nucleotide residues of the particular nucleic acid sequence as set forth herein which is subjected to comparison (e.g. a particular nucleic acid sequence characterized by a sequence identifier in the sequence listings).

As used herein, the term “homology” or “identity” refers to the identity of subunit sequence between two polymer molecules, for example, between two nucleic acid molecules, such as two DNA molecules or two RNA molecules, or between two polypeptide molecules. When subunit positions in two molecules are occupied by the same monomer subunit; for example, if the position of each of two DNA molecules is occupied by adenine, they are homologous or identical at that position. The homology between two sequences is a direct function of the number of matching or homologous positions; for example, if half of the positions in the two sequences (for example, 5 positions in a polymer of 10 subunits in length) are homologous, the two sequences are 50% homologous; if 90% of the positions (for example, 9 out of 10) are matched or homologous, then the two sequences are 90% homologous.

In the context of two or more nucleic acid or polypeptide sequences, identity percent refers to two or more sequences that are the same. When comparing and aligning for maximum correspondence in a comparison window or a designated area, as measured by using one of the following sequence comparison algorithms or by manual alignment and visual inspection, if the two sequences have a specified percentage of identical amino acid residues or nucleotides (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or if not specified, over the entire sequence), then the two sequences are “substantially the same”. Optionally, the identity exists over a region of at least about 50 nucleotides (or 10 amino acids) in length, or more preferably over a region of 100 to 500 or 1000 or more nucleotides in length (Or 20, 50, 200 or more amino acids). For sequence comparison, usually a sequence serves as a reference sequence against which the test sequence is compared. When a sequence comparison algorithm is used, a test sequence and a reference sequence are input into a computer, and the sub-sequence coordinates and the sequence algorithm program parameters are specified, if necessary. Default program parameters can be used, or alternative parameters can be specified. Subsequently, the sequence comparison algorithm calculates the percent sequence identity of the test sequence relative to the reference sequence based on the program parameters. Methods of sequence alignment for comparison are well known in the art.

“Coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region.

A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques well-known in the art (e.g., molecular cloning, polymerase chain reaction (PCR), digestion with restriction enzymes, in vitro ligation, mutagenesis, site-directed mutagenesis, prokaryotic and eukaryotic cell transformation or transduction, in vitro DNA/RNA synthesis, in vitro RNA-5′-capping, in vitro RNA-polyadenylation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3, 4th edition).

As used herein, the term “recombinant”, refers to any nucleic acid (e.g., DNA, or RNA), which is made by combining genetic material from two or more different sources (e.g., a protein coding sequence cloned in an expression vector), to any peptide (e.g., oligopeptide, polypeptide, or protein) which is encoded by such recombinant nucleic acids (e.g., a protein encoded by a nuclei acid comprised in an expression vector), and any cell or organism that comprises such recombinant nucleic acids and/or recombinant peptides (e.g., proteins). In some aspects, the recombinant nucleic acid, peptide, cell or organism comprises a portion of the genetic material from at least one source. In some aspects, “recombinant DNA” molecules can include DNA molecules derived from one organism and inserted in a host organism to produce new genetic combinations. In some aspects, “recombinant RNA” molecule (e.g., recombinant mRNA molecules) can include RNA molecules derived from one organism and inserted in a host organism to produce the expression of a desired genetic product in the host organism. In some aspects, “recombinant peptide” molecules (e.g., recombinant TCRs) can include amino acid molecules derived from or comprised in an organism or cell, which are encoded by (i.e., expressed from) a recombinant nucleic acid molecules.

A “recombinant TCR”, or an “engineered TCR” can comprise one or more of the same or similar structural properties (e.g., antigen binding specificity) as a TCR protein or portion thereof of an antigen-specific T cell. In some aspects, the TCR of the antigen-specific T cell recognizes a tumor antigen of interest (e.g., neoantigen). In some aspects of the present disclosure, the tumor antigen of interest is a Kirsten Rat Sarcoma Viral Oncogene Homolog (KRAS) protein comprising a G12V mutation. In some aspects of the present disclosure, the tumor antigen of interest is a Kirsten Rat Sarcoma Viral Oncogene Homolog (KRAS) protein comprising a G12V mutation, which comprises an epitope that is presented on human leukocyte antigen-A (HLA-A). In some aspects, the tumor antigen (e.g., neoantigen) of interest is presented on a specific MHC molecule (e.g., human leucocyte antigen-A). In some aspects of the present disclosure, the tumor antigen of interest is a Kirsten Rat Sarcoma Viral Oncogene Homolog (KRAS) protein comprising a G12V mutation, which comprises an epitope that is presented on human leukocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

As used herein, the term “antigen” or “Ag” refers to a molecule that causes an immune response. The immune response can involve the production of antibodies or the activation of cells with specific immunity, or both. A skilled person in the art should understand that macromolecules including proteins or peptides can serve as an antigen. In addition, an antigen can be derived from recombinant or genomic DNA. When the term is used herein, the term includes any DNA including a nucleotide sequence or part of a nucleotide sequence encoding a protein that causes an immune response, and therefore encoding an “antigen”. In addition, the antigen need not be encoded only by the full-length nucleotide sequence of the gene. It is obvious that the present invention includes, but is not limited to, the use of partial nucleotide sequences of more than one gene, and these nucleotide sequences are arranged in different combinations to encode polypeptides eliciting a desired immune response. Moreover, the antigen does not need to be encoded by a “gene” at all. The antigen can be produced synthetically, or it can be derived from a biological sample, or it can be a macromolecule other than a polypeptide. Such biological samples may include, but are not limited to, tissue samples, tumor samples, cells or fluids with other biological components.

The term “antigen recognition unit”, or “antigen-binding fragment” as used herein can include any molecular structure containing a polypeptide chain that has a specific shape that matches the epitope and recognizes the epitope, in which one or more non-covalent binding interactions stabilize the complex between the molecular structure and the epitope.

If the antigen recognition unit binds to an antigen with greater affinity or avidity compared with binding with other reference antigens (including polypeptides or other substances), the antigen recognition unit “specifically binds” to the antigen or is “immunoreactive with the antigen”.

As used herein, the term “tumor antigen” refers to an antigen common to specific hyperproliferative diseases. In certain aspects, the hyperproliferative disorder antigens of the disclosure are derived from cancer, e.g., NSCLC. As used herein, the term “neoantigen”, or “tumor-specific antigen”, or “TSA”, refers to a type of tumor antigens derived from non-synonymous mutations. Neoantigens may originate from, for example, non-synonymous single nucleotide variants (SNVs), DNA insertions or deletions, gene fusion events, splice-site creation mutations (SCMs), mRNA intron retention, and endogenous retroelements.

As used herein, the term “cancer” refers to a broad group of various diseases characterized by the uncontrolled growth of abnormal cells (e.g., malignant cells) in the body. Unregulated cell division and growth results in the formation of malignant tumors that invade neighboring tissues through local spread and can also metastasize to distant parts of the body through the lymphatic system or bloodstream. In some aspects, the methods of the present disclosure can be used to reduce the size of a primary tumor or a metastatic tumor, or treat a primary tumor or a metastatic tumor. The conditions that can be treated or prevented by the method of the present invention include, for example, various neoplasms, including benign or malignant tumors, various hyperplasias, and the like. The method of the present disclosure can achieve the inhibition and/or reversal of the undesirable hyperproliferative cell growth involved in such conditions. In some aspects, the cancer can be lung cancer, e.g., non-small cell lung cancer (NSCLC).

The term “tumor” as used herein refers to any mass of tissue that results from excessive cell growth or proliferation, either benign (non-cancerous) or malignant (cancerous), including pre-cancerous lesions.

The term “primary tumor”, as used herein, refers to the original, or first, tumor formed in the subject's body.

The term “metastasis”, “metastatic”, “secondary tumor”, or “metastatic tumor”, as used herein, refer to cancer (e.g., a tumor) formed by cancer cells derived from a primary cancer (e.g., tumor) that spread to further locations or areas of the body.

As used herein, “refractory” as used herein refers to a disease, such as cancer, which does not respond to treatment. In an embodiment, a refractory cancer may be resistant to treatment before or at the beginning of the treatment. In other embodiments, a refractory cancer may become resistant during treatment. Refractory cancers are also called resistant cancers. In some aspects, refractory or recurrent malignant tumors can use the treatment methods disclosed herein.

As used herein, “relapsed” as used herein refers to the return of the signs and symptoms of a disease (e.g. cancer) or the return of a disease such as cancer during a period of improvement, for example, after a therapy, such as a previous treatment of cancer therapy.

As used herein, the term “administration” refers to the administration of a composition of the present disclosure to a subject or system. Administration to an animal subject (e.g., to a human) can be by any appropriate route. “Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

As used herein, the terms “effective amount,” “therapeutically effective amount,” and a “sufficient amount” refer to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system, and, as such, an “effective amount” depends on the context in which it is being applied. In some aspects, an “effective amount”, or synonym thereto, of an agent is an amount that results in a beneficial or desired result in a subject as compared to a control, e.g., a beneficial result can take the form of an improvement over baseline (i.e., an improvement over a measurement or observation made prior to initiation of therapy according to the method). When indicating “immunologically effective amount”, “anti-tumor effective amount”, “tumor-suppressing effective amount” or “therapeutically effective amount”, the precise number of immune effector cells and therapeutic agents of the present invention to be administered can be determined by a physician in consideration of the individual's age, weight, tumor size, degree of infection or metastasis, and the condition of a patient (subject). An effective amount of immune effector cells refers to, but is not limited to, the number of immune effector cells which can increase, enhance or prolong the anti-tumor activity of immune effector cells; increase the number of anti-tumor immune effector cells or activated immune effector cells; promote tumor regression, tumor shrinkage and/or tumor necrosis.

The term “prophylactically effective amount,” as used herein, includes the amount of an agent, (e.g., a modified T cell or composition thereof disclosed herein) that, when administered to a subject having or predisposed to have a disease or disorder (e.g., cancer) is sufficient to prevent, reduce the symptoms of, or ameliorate the disease or disorder or one or more symptoms of the disease or disorder. Ameliorating the disease or disorder includes slowing the course of the disease or disorder or reducing the severity of later-developing disease or disorder. The “prophylactically effective amount” can vary depending on the characteristics of the agent, e.g., modified T cell, or composition of the disclosure, how the agent is administered, the degree of risk of disease, and the history, age, weight, family history, genetic makeup, the types of preceding or concomitant treatments, if any, and other individual characteristics of the patient to be treated.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”, “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the terms “individual” and “subject” have the same meaning herein, and can be a human and animal from other species. As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. In some aspects, the patient is a subject who has a disease, disorder, or condition, or is at risk of suffering from a disease, disorder, or condition, or is otherwise in need of the compositions and methods provided herein.

As used herein, the term “immune response” refers to a biological response within an organism against a foreign agent or abnormal cell (e.g., a tumor cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, cancerous or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4+ or CD8+ T cell, or the inhibition of a regulatory T cell (Treg cell).

As used herein, the term “immune effector cell” refers to a cell that exerts an effector function during an immune response, including, for example, immune cells secreting cytokines and/or chemokines, killing microorganisms, secreting antibodies, and recognizing or eliminating tumor cells. In some embodiments, immune effector cells include T cells (cytotoxic T cells, helper T cells, and tumor-infiltrating T cells), B cells, natural killer cells, neutrophils, macrophages, and dendritic cells.

As used herein, the term “immune effector function” includes any function mediated by the composition of the immune system, which can lead to inhibition of tumor growth and/or inhibition of tumorigenesis, including inhibition the spread and metastasis of a tumor. Preferably, the immune effector function kills tumor cells.

As used herein, the term “antigen presenting cell” or “APC” refers to a cell in the immune system that display a complex of foreign antigens and major histocompatibility complex (MHC) on the surface, such as helper cells (such as B cells, dendritic cells, etc.). T cells can recognize these complexes using a T cell receptor (TCR) thereof. An APC can process the antigen and present it to T cells.

As used herein, the term “immunotherapy” refers to the treatment of a disease by inducing, enhancing, suppressing or otherwise modifying an immune response.

As used herein, the term “autologous” refers to any material derived from an individual that will later be reintroduced into that same individual.

As used herein, the term “allogeneic” refers to any material derived from a different animal or a different patient of the same species as the individual into which the material is introduced. When the genes at one or more loci are different, two or more individuals are considered to be allogeneic to each other. In some aspects, allogeneic materials from individuals of the same species may be genetically different enough for antigenic interaction to occur.

As used herein, the term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present disclosure to prevent or reduce the frequency of tumorigenesis.

The term “T cell receptor” or “TCR”, also referred to herein as a “TCR protein”, “TCR portion” or “TCR unit” is a protein complex found on the surface of all T cells. In some aspects, a TCR can bind to CD3 by a non-covalent bond to form a TCR-CD3 complex. TCRs can recognize antigens bound to major histocompatibility complex molecules. TCRs are heterodimers including two different peptide chains. There are two categories of TCRs: TCR1 and TCR2. TCR1 is composed of peptide chains gamma (γ) and delta (δ); and TCR2 is composed of peptide chains alpha (α) and beta (β). Each peptide chain can be divided into several parts, such as variable region (V region), constant region (C region), transmembrane region and cytoplasmic region, characterized in that the cytoplasmic region is typically short in length. TCR molecules are immunoglobulins, and their antigen specificity resides in the V regions (e.g., Vα and Vβ), each of which has three hypervariable regions CDR1, CDR2, and CDR3, in which CDR3 has the largest variation and determines the binding specificity of the TCR to an antigen. When a TCR recognizes a MHC-antigen peptide complex, CDR1 and CDR2 recognize and bind to the side wall of the antigen binding groove of the MHC molecule, and CDR3 binds to the antigen peptide. In some aspects, a partial genetic modification can be used to improve the “affinity” and effectiveness of TCRs to target, thereby providing a high-affinity TCR. Unless otherwise stated, the term “TCR” should be understood to encompass functional TCR fragments thereof. The term also encompasses intact or full-length TCRs, including TCRs in the αβ form or γδ form.

As used herein, a “TCR T cell” or “engineered TCR T cell” is a T cell that has been transduced with (for example, according to the methods disclosed herein) and expresses a recombinant TCR, or antigen-binding fragment thereof. In some aspects, the T cell is a CD4+ T cell, CD8+ T cell, or CD4+/CD8+ T cell. In some aspects, the T cell is a natural killer (NK) cell.

As used herein, the terms “variable region” or “variable domain” are used interchangeably and are common in the art. The variable region typically refers to a portion of an immunoglobulin molecule, generally, a portion of a light or heavy chain, which differ in sequence among immunoglobulin molecule and are used in the binding and specificity of a particular immunoglobulin molecule for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the immunoglobulin molecule with antigen.

The “hypervariable regions” in each chain are held together in close proximity by FRs, and with the hypervariable regions from the other chain, contribute to the formation of the antigen-binding site of immunoglobulin molecule (see Kabat et al, Sequences of Proteins of Immunological Interest, 1992). The hypervariable region generally comprises amino acid residues from a “complementary determining region” or “CDR”, the latter being of highest sequence variability and/or involved in antigen recognition. A number of CDR definitions are in use and are encompassed herein. The Kabat definition is based on sequence variability and is the most commonly used (Kabat E A et al., supra). Chothia refers instead to the location of the structural loops (Chothia C & Lesk A M (1987) J. Mol. Biol. 196: 901-917). The AbM definition is a compromise between the Kabat and the Chothia definitions and is used by Oxford Molecular's AbM antibody modelling software (Martin A C R et al., (1989) Proc. Natl Acad. Sci. USA, 86: 9268-72; Martin A C R et al., (1991) Methods Enzymol. 203: 121-153; Pedersen J T et al., (1992) Immunomethods, 1: 126-136; Rees A R et al., (1996) In Sternberg M. J. E. (ed.), Protein Structure Prediction. Oxford University Press, Oxford, 141-172). The contact definition has been recently introduced (Maccallum R M et al., (1996) J. Mol. Biol. 262: 732-7 45) and is based on an analysis of the complex structures available in the Protein Databank. The definition of the CDR by IMGT®, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27(1): 209-12; Ruiz M et al., (2000) Nucleic Acids Res. 28(1): 219-21; Lefranc M P (2001) Nucleic Acids Res. 29(1): 207-9; Lefranc M P (2003) Nucleic Acids Res. 31(1): 307-10; Lefranc M P et al., (2005) Dev. Comp. Immunol. 29(3): 185-203; Kaas Q et al., (2007) Briefings in Functional Genomics & Proteomics, 6(4): 253-64).

In some aspects, the Complementarity Determining Regions (CDRs) disclosed herein can be defined according to IMGT®. In some aspects, the CDRs can be defined according to Chothia. In some aspects, the CDRs can be defined according to Kabat.

In some aspects, alternate CDR sequences are provided for the same framework amino acid sequences of a variable region. In some aspects, the alternate CDR sequences are generated using software programs. In some aspects, different software can be used to generate alternate CDR sequences for the framework sequences of a variable region with different CDR sequences resulting from the use of the different software programs. In some aspects, the use of alternate CDR sequences can improve binding affinities of a bispecific antibody molecule to at least one antigen. In some aspects, alternate CDR sequences are used for affinity optimization of one or both antigen binding sites of a bispecific antibody molecule according to the present invention. In some aspects, the alternate CDRs are defined according to Kabat, Chothia, Paratome, AbM, Contact and/or IMGT annotations. In some aspects, the CDRs are defined according to more than one annotation.

In some aspects, the CDRs of a recombinant TCRs, or antigen-binding fragment thereof, can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). As used herein, the terms “constant region” and “constant domain” are interchangeable and have their meaning common in the art. The constant region of an immunoglobulin molecule generally has a more conserved amino acid sequence relative to an immunoglobulin variable domain.

As used herein, an “epitope” is a term in the art and refers to a localized region of an antigen to which a TCR, or antigen-binding region thereof, can bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, the epitope to which a TCR binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al, (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al, (2000) Acta Crystallogr D Biol Crystallogr 56(Pt 10): 1316-1323). Mutagenesis mapping studies can be accomplished using any method known to one of skill in the art. See, e.g., Champe M et al, (1995) J Biol Chem 270: 1388-1394 and Cunningham B C & Wells J A (1989) Science 244: 1081-1085 for a description of mutagenesis techniques, including alanine scanning mutagenesis techniques.

As used herein, the term “direct binding” refers to an antigen binding molecule that recognizes and binds a protein of a binding partner (such as a tumor antigen).

As used herein, the term “Kirsten Rat Sarcoma Viral Oncogene Homolog”, or “KRAS” (chromosome 12p12.1) refers to member of the RAS canonical family of genes that also includes HRAS (chromosome 11p15.5) and NRAS (chromosome 1p13.1). The three genes are highly conserved across different species and encode monomeric GTPases that cycle between active (GTPbound) and inactive (GDPbound) states in response to extracellular cues. Unlike HRAS and NRAS, KRAS undergoes alternative splicing, resulting in two proteins (KRAS4A and KRAS4B) that differ only at their carboxyl termini. RAS proteins are 188/189 amino acids in length, and the sequence of the first 165 amino acids is almost identical. This region contains highly conserved domains that are responsible for GTP binding and hydrolysis and functional interactions with regulators and downstream effectors. The hypervariable carboxyl domain is the most distinguishing feature among the RAS family members and contains sequences important for determining posttranslational modification, including the terminal CAAX domain that is responsible for membrane targeting. The importance of RAS in cancer pathogenesis was first recognized more than three decades ago when it was discovered that mutated versions of KRAS and HRAS were responsible for the transforming activities of sarcoma-inducing retroviruses in rats. We now know that somatic activating mutations in the cellular homologs of all three RAS family members occur in a wide spectrum of human cancers. Protein mutational activation of KRAS is a common oncogenic event especially in lung cancer and other epithelial cancer types. Efforts to develop therapies that counteract the oncogenic effects of mutant KRAS have been largely unsuccessful, and cancers driven by mutant KRAS remain among the most refractory to available treatments. (Westcott et al., The genetics and biology of KRAS in lung cancer, Chin J Cancer, 2013, 32(2):63-70). These mutations predominantly occur at codons 12, 13, and 61, and result in constitutive activation of RAS. Overall, RAS mutations have been found in approximately 30% of all human cancers, with KRAS as the most commonly mutated family member. Specifically, the G12V mutation is expressed by 25% of NSCLC patients with KRAS mutations.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### Recombinant T Cell Receptors (TCRs)

Certain aspects of the disclosure are directed to recombinant T cell receptors (TCRs), or antigen-binding fragments thereof, capable of binding to an epitope of a Kirsten Rat Sarcoma Viral Oncogene Homolog (KRAS) protein comprising a G12V mutation. In some aspects, the recombinant T cell receptors (TCRs), or antigen-binding fragments thereof, are capable of binding to an epitope of a Kirsten Rat Sarcoma Viral Oncogene Homolog (KRAS) protein comprising a G12V mutation presented on a human leucocyte antigen-A (HLA-A). In some aspects, the recombinant T cell receptors (TCRs), or antigen-binding fragments thereof, capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the (HLA-A)*03:01 allele presents the epitope comprising the amino acid sequence of VVGAVGVGK (SEQ ID NO: 1). In some aspects, the (HLA-A)*03:01 allele presents the epitope comprising the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the recombinant TCRs of the present disclosure include the variable chain portions of the TCR or any functional fragment thereof, such as an antigen-binding portion of a TCR (e.g., antigen-binding fragments) that binds to a specific antigenic peptide bound in an MHC molecule, i.e. MHC-peptide complex. In some aspects, the antigen-binding portion or antigen-binding fragment of a TCR, refers to a molecule that contains a portion of the structural domains of a TCR, but that binds the antigen (e.g. MHC-peptide complex) to which the full TCR binds. In some cases, an antigen-binding portion contains the variable domains of a TCR, such as a variable alpha (α) chain and a variable beta (p) chain, or a variable gamma (γ) chain and a variable delta (δ) chain of a TCR, sufficient to form a binding site for binding to a specific MHC-peptide complex, such as generally where each chain contains three complementarity determining regions (CDRs).

In some aspects, a recombinant TCR of the present disclosure, or antigen-binding fragment thereof, comprises a variable region (V region), a constant region (C region), a transmembrane region, a cytoplasmic region, or any combination thereof. In some aspects, a recombinant TCR of the present disclosure comprises a variable region (V region), a constant region (C region), a transmembrane region, and a cytoplasmic region.

In some aspects, the recombinant TCR of the present disclosure, or antigen-binding fragment thereof, is a TCR1. In some aspects, the recombinant TCR of the present disclosure, or antigen-binding fragment thereof, comprises a chain gamma (γ) and a chain delta (δ), or antigen-binding fragments thereof. In some aspects, the recombinant TCR of the present disclosure, or antigen-binding fragment thereof, comprises a variable chain gamma (γ) and a variable chain delta (δ), or antigen-binding fragments thereof. In some aspects, a recombinant TCR of the present disclosure comprises a full length chain gamma (γ) and a full length chain delta (δ).

In some aspects, the recombinant TCR of the present disclosure, or antigen-binding fragment thereof, is a TCR2. In some aspects, the recombinant TCR of the present disclosure, or antigen-binding fragment thereof, comprises a chain gamma (α) and a chain delta (β), or antigen-binding fragments thereof. In some aspects, the recombinant TCR of the present disclosure, or antigen-binding fragment thereof, comprises a variable chain gamma (α) and a variable chain delta (β), or antigen-binding fragments thereof. In some aspects, the recombinant TCR of the present disclosure comprises a full length chain gamma (α) and a full length chain delta (β).

In some aspects, a recombinant TCR of the present disclosure, or antigen-binding fragments thereof comprises a TCR α and a TCR β chain (or antigen-binding fragments thereof), or a TCR γ and a TCR δ chain (or antigen-binding fragments thereof), wherein the two chains (or antigen-binding fragments thereof) are present in a physical association with one another (e.g., in a complex) and are non-covalently joined to one another, or wherein the two chains (or antigen-binding fragments thereof) are distinct polypeptides but are covalently joined to one another, such as by a disulfide or other covalent linkage that is not a peptide bond. Other suitable linkages can comprise, for example, substituted or unsubstituted polyalkylene glycol, and combinations of ethylene glycol and propylene glycol in the form of, for example, copolymers. In other aspects, two polypeptides that constitute a TCR α and a TCR β chain or antigen-binding fragments thereof, or two polypeptides that constitute a TCR γ and a TCR δ chain or antigen-binding fragments thereof, can both be included in a single polypeptide, such as a fusion protein. In other aspects, two polypeptides that constitute a TCR α and a TCR β chain or antigen-binding fragments thereof, or two polypeptides that constitute a TCR γ and a TCR δ chain or antigen-binding fragments thereof can be included as separate polypeptides expressed on a T cell.

In some aspects, the variable domains of the TCR chains associate to form loops, or complementarity determining regions (CDRs) analogous to immunoglobulins, which confer antigen recognition and determine peptide specificity by forming the binding site of the TCR molecule and determine peptide specificity.

Typically, TCRs that exist in αβ and γδ forms are generally structurally similar, but T cells expressing them may have distinct anatomical locations or functions. The vast majority of T lymphocytes (over 90%) carry TCR2. Few T-lymphocytes with TCR1 are located beneath the mucous membranes and skin. A TCR can be found anchored to the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR can contain a variable domain, a constant domain, a transmembrane domain and/or a short cytoplasmic tail (see, e.g., Janeway et al, Immunobiology: The Immune System in Health and Disease, 3rd Ed., Current Biology Publications, p. 433, 1997). For example, in some aspects, each chain of the TCR can possess one N-terminal immunoglobulin variable domain, one immunoglobulin constant domain, a transmembrane region, and a short cytoplasmic tail at the C-terminal end. In some aspects, a TCR is associated with invariant proteins of the CD3 complex involved in mediating signal transduction.

In some aspects, the recombinant TCR of the present disclosure binds an epitope of a Kirsten Rat Sarcoma Viral Oncogene Homolog (KRAS) protein comprising a G12V mutation. In some aspects, the recombinant TCR of the present disclosure binds an epitope of a Kirsten Rat Sarcoma Viral Oncogene Homolog (KRAS) protein comprising a G12V mutation presented on a human leucocyte antigen-A (HLA-A). In some aspects, the recombinant TCR of the present disclosure binds an epitope of a Kirsten Rat Sarcoma Viral Oncogene Homolog (KRAS) protein comprising a G12V mutation presented on a human leucocyte antigen-A (HLA-A)*03:01.

In some aspects, the recombinant TCR of the present disclosure comprises a target-specific binding element, which is also referred to as an antigen recognition portion. For example, the antigen recognition portion can be selected to bind the target antigen as a cell surface marker associated with a specific disease state on the target cell (e.g., a neoantigen). In some aspects, the antigen recognition portion binds an epitope of a Kirsten Rat Sarcoma Viral Oncogene Homolog (KRAS) protein comprising a G12V mutation. In some aspects, the antigen recognition portion binds an epitope of a Kirsten Rat Sarcoma Viral Oncogene Homolog (KRAS) protein comprising a G12V mutation presented on a human leucocyte antigen-A (HLA-A). In some aspects, the antigen recognition portion binds an epitope of a Kirsten Rat Sarcoma Viral Oncogene Homolog (KRAS) protein comprising a G12V mutation presented on a human leucocyte antigen-A (HLA-A)(e.g., a HLA-A*03:01). In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the recombinant TCRs of the present disclosure specifically bind the epitope.

In some aspects, the recombinant TCRs of the disclosure, or the antigen-binding fragments thereof, can be derived from natural sources or from recombinant sources. In some aspects, the recombinant TCR, or the antigen-binding fragment thereof can be derived from any protein, especially a membrane-bound or transmembrane protein. In some aspects, the recombinant TCR of the disclosure, or the antigen-binding fragment thereof, is derived from a tumor antigen-specific T cell. In some aspects, the antigen-binding domain can associate with the transmembrane domain. In some aspects, antigen-binding fragments can particularly useful in the present invention.

In some aspects, the CDRs of a recombinant TCR, or antigen-binding fragment thereof, can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, CDR1B is at positions 26 to 35, CDR2B is at positions 51 to 57, CDR3β is at positions 93 to 102, CDR1A is at positions 27 to 32, CDR2A is at positions 50 to 52, and CDR3A is at positions 89 to 97. In some aspects, provided herein are recombinant TCRs and antigen-binding fragments thereof that bind to an epitope of a Kirsten Rat Sarcoma Viral Oncogene Homolog (KRAS) protein comprising a G12V mutation presented on a human leucocyte antigen-A (HLA-A)*03:01 and comprise the α and β CDRs of a TCR sequence disclosed herein, e.g., shown in Table 1, determined by IMGT method, for example, as described in Lefranc M-P (1999) supra and Lefranc M-P et al., (1999) supra). In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the CDRs of recombinant TCRs, or of the antigen-binding fragment thereof, can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are recombinant TCRs and antigen-binding fragments thereof that bind to an epitope of a Kirsten Rat Sarcoma Viral Oncogene Homolog (KRAS) protein comprising a G12V mutation presented on a human leucocyte antigen-A (HLA-A) (e.g., a HLA-A*03:01) and comprise the α and β CDRs of a TCR sequence disclosed herein, e.g., shown in Table 1, determined by the numbering method of MacCallum R M et al. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the CDRs of the recombinant TCRs, or of the antigen-binding fragment thereof, can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are TCRs and antigen-binding fragments thereof that bind to an epitope of a Kirsten Rat Sarcoma Viral Oncogene Homolog (KRAS) protein comprising a G12V mutation presented on a human leucocyte antigen-A (HLA-A)*03:01 and comprise the α and β CDRs of a TCR sequence disclosed herein, e.g., shown in Table 1, determined by AbM numbering method. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the CDRs of the recombinant TCRs, or of the antigen-binding domain thereof that bind to an epitope of a Kirsten Rat Sarcoma Viral Oncogene Homolog (KRAS) protein comprising a G12V mutation presented on a human leucocyte antigen-A (HLA-A)*03:01, provided herein, are determined by the IMGT numbering scheme. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the recombinant T-cell Receptor (TCR), or the antigen binding domain thereof, comprises: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68. In some aspects, the recombinant TCR binds an epitope comprising the amino acid sequence of VVGAVGVGK (SEQ ID NO: 1) of a Kirsten Rat Sarcoma Viral Oncogene Homolog (KRAS) protein comprising a G12V mutation presented on a human leucocyte antigen-A (HLA-A)*03:01. In some aspects, the recombinant TCR, or the antigen-binding domain thereof, binds an epitope comprising the amino acid sequence of VVVGAVGVGK (SEQ ID NO: 2) of a KRAS protein comprising a G12V mutation presented on a human leucocyte antigen-A (HLA-A)*03:01. In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the TCR alpha chain comprises CDR1α, CDR2α, and CDR3α of SEQ ID NO: 15, 31 or 63. In some aspects, the TCR alpha chain comprises CDR1α, CDR2α, and CDR3α of SEQ ID NO: 16, 32, or 64. In some aspects, the TCR beta chain comprises CDR1β, CDR2β, and CDR3β of SEQ ID NO: 17, 35, or 67. In some aspects, the TCR beta chain comprises CDR1β, CDR2β, and CDR3β of SEQ ID NO: 18, 36, or 68.

In some aspects, the recombinant T cell receptor (TCR), or the antigen binding domain thereof, capable of binding to an epitope of a Kirsten Rat Sarcoma Viral Oncogene Homolog (KRAS) protein comprising a G12V mutation presented on a human leucocyte antigen-A (HLA-A)*03:01, comprises: (a) a TCR-alpha chain comprising (i) a variable TCR alpha chain complementarity determining region 1 (CDR1α) having at least 85%, at least 90%, at least 91%, at least 92%, at least 93%, at least 94%, at least 95%, at least 96%, at least 97%, at least 98%, at least 99%, sequence identity to SEQ ID NO: 3, or 4, (ii) a variable TCR alpha chain complementarity determining region 2 (CDR2α) having at least 85%, at least 90%, at least 91%, at least 92%, at least 93%, at least 94%, at least 95%, at least 96%, at least 97%, at least 98%, at least 99%, sequence identity to SEQ ID NO: 5, or 6, (iii) a variable TCR alpha chain complementarity determining region 3 (CDR3α) having at least 85%, at least 90%, at least 91%, at least 92%, at least 93%, at least 94%, at least 95%, at least 96%, at least 97%, at least 98%, at least 99%, sequence identity to SEQ ID NO: 7, or 8, and (b) a TCR beta chain comprising (iv) a variable TCR beta chain complementarity determining region 1 (CDR1β) having at least 85%, at least 90%, at least 91%, at least 92%, at least 93%, at least 94%, at least 95%, at least 96%, at least 97%, at least 98%, at least 99%, sequence identity to SEQ ID NO: 9, or 10, (v) a variable TCR beta chain complementarity determining region 2 (CDR2β) having at least 85%, at least 90%, at least 91%, at least 92%, at least 93%, at least 94%, at least 95%, at least 96%, at least 97%, at least 98%, at least 99%, sequence identity to SEQ ID NO: 11, or 12, (vi) variable TCR beta chain complementarity determining region 3 (CDR3β) having at least 85%, at least 90%, at least 91%, at least 92%, at least 93%, at least 94%, at least 95%, at least 96%, at least 97%, at least 98%, at least 99%, sequence identity to SEQ ID NO: 13, or 14.

In some aspects, (i) the CDR1α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 3, (ii) the CDR2α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 5, (iii) the CDR3α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 7, (iv) the CDR1β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 9, (v) the CDR2β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 11, (vi) the CDR3β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 13.

In some aspects, (i) the CDR1α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 4, (ii) the CDR2α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 6, (iii) the CDR3α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 8, (iv) the CDR1β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 10, (v) the CDR2β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 12, (vi) the CDR3β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 14. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the recombinant TCR is capable of direct binding to the epitope. In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the recombinant T cell receptors (TCRs), or the antigen-binding fragment thereof, comprises (a) an alpha chain (TCR-α comprising (i) a variable TCR alpha chain complementarity determining region 1 (CDR1 α) comprising the amino acid sequence of SEQ ID NO: 3, or 4, (ii) a variable TCR alpha chain complementarity determining region 2 (CDR2 α) comprising the amino acid sequence of SEQ ID NO: 5, or 6, (iii) a variable TCR alpha chain complementarity determining region 3 (CDR3 α) comprising the amino acid sequence of SEQ ID NO: 7, or 8; and (b) a beta chain (TCR-β comprising (i) a variable TCR beta chain complementarity determining region 1 (CDR1 β) comprising the amino acid sequence of SEQ ID NO: 9, or 10, (ii) a variable TCR beta chain complementarity determining region 2 (CDR2 β) comprising the amino acid sequence of SEQ ID NO: 11, or 12, (iii) variable TCR beta chain complementarity determining region 3 (CDR3 β) comprising the amino acid sequence of SEQ ID NO: 13, or 14. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the recombinant T cell receptors (TCRs), or the antigen-binding fragment thereof, of the disclosure, comprise an alpha chain (TCR-α) and a beta chain (TCR-β). In some aspects, the TCR-α comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64, and the TCR-β comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68.

In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31 or 63. In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 17, 35, or 67. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 18, 36, or 68.

In some aspects, the recombinant T cell receptors (TCRs), or the antigen-binding fragment thereof, of the disclosure, comprise a TCR-α comprising an amino acid sequence of SEQ ID NO: 15, 16, 31, 32, 63, or 64, and a TCR-β comprising an amino acid sequence of SEQ ID NO: 17, 18, 35, 36, 67, or 68. In some aspects, the TCR alpha chain comprises an amino acid sequence of SEQ ID NO: 15, 31 or 63. In some aspects, the TCR alpha chain comprises an amino acid sequence of SEQ ID NO: 16, 32, or 64. In some aspects, the TCR beta chain comprises an amino acid sequence of SEQ ID NO: 17, 35, or 67. In some aspects, the TCR beta chain comprises an amino acid sequence of SEQ ID NO: 18, 36, or 68.

In some aspects, the recombinant T-cell Receptor (TCR) comprises: (a) a TCR alpha chain comprising (i) a variable TCR alpha chain complementary determining region 1 (CDR1α) which is encoded by a nucleic acid sequence of SEQ ID NO: 71, 72, 33, 34, 65, or 66, (ii) a CDR2α which is encoded by a nucleic acid sequence of SEQ ID NO: 71, 72, 33, 34, 65, or 66, and (iii) a CDR3α which is encoded by a nucleic acid sequence of SEQ ID NO: 71, 72, 33, 34, 65, or 66; and (b) a TCR beta chain comprising (i) a variable TCR beta chain complementary determining region 1 (CDR1β) which is encoded by a nucleic acid sequence of SEQ ID NO: 73, 74, 37, 38, 69, or 70, (ii) a CDR2β which is encoded by a nucleic acid sequence of SEQ ID NO: 73, 74, 37, 38, 69, or 70, and (iii) a CDR3β which is encoded by a nucleic acid sequence of SEQ ID NO: 73, 74, 37, 38, 69, or 70. In some aspects, the recombinant TCR binds an epitope comprising the amino acid sequence of VVGAVGVGK (SEQ ID NO: 1) of a Kirsten Rat Sarcoma Viral Oncogene Homolog (KRAS) protein comprising a G12V mutation presented on a human leucocyte antigen-A (HLA-A)*03:01. In some aspects, the recombinant TCR binds an epitope comprising the amino acid sequence of VVVGAVGVGK (SEQ ID NO: 2) of a KRAS protein comprising a G12V mutation presented on a human leucocyte antigen-A (HLA-A)*03:01. In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, (i) the CDR1α is encoded by a nucleic acid sequence of SEQ ID NO: 71, 33, or 65, the (ii) CDR2α is encoded by a nucleic acid sequence of SEQ ID NO: 71, 33, or 65, (iii) the CDR3α is encoded by a nucleic acid sequence of SEQ ID NO: 71, 33, or 65; (iv) the CDR1β is encoded by a nucleic acid sequence of SEQ ID NO: 73, 37, or 69, (v) the CDR2β is encoded by a nucleic acid sequence of SEQ ID NO: 73, 37, or 69, and (vi) the CDR3β is encoded by a nucleic acid sequence of SEQ ID NO: 73, 37, or 69.

In some aspects, (i) the CDR1α is encoded by a nucleic acid sequence of SEQ ID NO: 72, 34, or 66, the (ii) CDR2α is encoded by a nucleic acid sequence of SEQ ID NO: 72, 34, or 66, (iii) the CDR3α is encoded by a nucleic acid sequence of SEQ ID NO: 72, 34, or 66; (iv) the CDR1β is encoded by a nucleic acid sequence of SEQ ID NO: 74, 38, or 70, (v) the CDR2β is encoded by a nucleic acid sequence of SEQ ID NO: 74, 38, or 70, and (vi) the CDR3β is encoded by a nucleic acid sequence of SEQ ID NO: 74, 38, or 70.

In some aspects, the recombinant T cell receptor (TCR) capable of binding to an epitope of a Kirsten Rat Sarcoma Viral Oncogene Homolog (KRAS) protein comprising a G12V mutation presented on a human leucocyte antigen-A (HLA-A)*03:01, comprises: (a) a TCR-alpha chain comprising (i) a variable TCR alpha chain complementarity determining region 1 (CDR1α) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 19, or 20, (ii) a variable TCR alpha chain complementarity determining region 2 (CDR2α) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 21, or 22, (iii) a variable TCR alpha chain complementarity determining region 3 (CDR3α) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 23, or 24, and (b) a TCR beta chain comprising (iv) a variable TCR beta chain complementarity determining region 1 (CDR1β) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 25, or 26, (v) a variable TCR beta chain complementarity determining region 2 (CDR2β) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 27, or 28, (vi) variable TCR beta chain complementarity determining region 3 (CDR3β) which is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 29, or 30. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the recombinant TCR is capable of direct binding to the epitope. In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, (i) the CDR1α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 19, (ii) the CDR2α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 21, (iii) the CDR3α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 23, (iv) the CDR1β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 25, (v) the CDR2β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 27, (vi) the CDR3β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 29.

In some aspects, (i) the CDR1α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 20 (ii) the CDR2α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 22, (iii) the CDR3α is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 24, (iv) the CDR1β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 26, (v) the CDR2β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 28, (vi) the CDR3β is encoded by a nucleic acid sequence having at least 85%, at least 90%, at least 91%, at least 92%, at least 93%, at least 94%, at least 95%, at least 96%, at least 97%, at least 98%, at least 99%, sequence identity to SEQ ID NO: 30.

In some aspects, the recombinant T cell receptors (TCRs), or the antigen-binding fragment thereof, of the disclosure, comprises (a) an alpha chain (TCR-α) comprising (i) a variable TCR alpha chain complementarity determining region 1 (CDR1 α) which is encoded by a nucleic acid sequence comprising SEQ ID NO: 19, or 20, (ii) a variable TCR alpha chain complementarity determining region 2 (CDR2 α) which is encoded by a nucleic acid sequence comprising SEQ ID NO: 21, or 22, (iii) a variable TCR alpha chain complementarity determining region 3 (CDR3 α) which is encoded by a nucleic acid sequence comprising SEQ ID NO: 23 or 24; and (b) a beta chain (TCR-β) comprising (i) a variable TCR beta chain complementarity determining region 1 (CDR1 β) which is encoded by a nucleic acid sequence comprising SEQ ID NO: 25, or 26, (ii) a variable TCR beta chain complementarity determining region 2 (CDR2 β) which is encoded by a nucleic acid sequence comprising SEQ ID NO: 27, or 28, (iii) variable TCR beta chain complementarity determining region 3 (CDR3 β) which is encoded by a nucleic acid sequence comprising SEQ ID NO: 29, or 30.

In some aspects, (i) the CDR1α is encoded by a nucleic acid sequence of SEQ ID NO: 19, (ii) the CDR2α is encoded by a nucleic acid sequence of SEQ ID NO: 21, (iii) the CDR3α is encoded by a nucleic acid sequence of SEQ ID NO: 23, (iv) the CDR1β is encoded by a nucleic acid sequence of SEQ ID NO: 25, (v) the CDR2β is encoded by a nucleic acid sequence of SEQ ID NO: 27, (vi) the CDR3β is encoded by a nucleic acid sequence of SEQ ID NO: 29.

In some aspects, (i) the CDR1α is encoded by a nucleic acid sequence of SEQ ID NO: 20 (ii) the CDR2α is encoded by a nucleic acid sequence of SEQ ID NO: 22, (iii) the CDR3α is encoded by a nucleic acid sequence of SEQ ID NO: 24, (iv) the CDR1β is encoded by a nucleic acid sequence of SEQ ID NO: 26, (v) the CDR2β is encoded by a nucleic acid sequence of SEQ ID NO: 28, (vi) the CDR3β is encoded by a nucleic acid sequence of SEQ ID NO: 30.

In some aspects, the recombinant T cell receptors (TCRs), or the antigen-binding fragment thereof, of the disclosure, comprise an alpha chain (TCR-α) and a beta chain (TCR-β). In some aspects, the TCR-α comprises an amino acid sequence which is encoded by a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 72, 33, 34, 65, or 66, and the TCR-β comprises an amino acid sequence which is encoded by a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 74, 37, 38, 69, or 70.

In some aspects, the TCR-α comprises an amino acid sequence which is encoded by a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 33, or 65, and the TCR-β comprises an amino acid sequence which is encoded by a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 37, or, 69.

In some aspects, the TCR-α comprises an amino acid sequence which is encoded by a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 72, 34, or 66, and the TCR-β comprises an amino acid sequence which is encoded by a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 74, 38, or 70.

In some aspects, the recombinant T cell receptors (TCRs), or the antigen-binding fragment thereof, of the disclosure, comprise a TCR-α comprising an amino acid sequence which is encoded by a nucleic acid sequence of SEQ ID NO: 71, 72, 33, 34, 65, or 66, and a TCR-β comprising an amino acid sequence which is encoded by a nucleic acid sequence of SEQ ID NO: 73, 74, 37, 38, 69, or 70.

In some aspects, the recombinant T cell receptors (TCRs), or the antigen-binding fragment thereof, of the disclosure, comprise a TCR-α comprising an amino acid sequence which is encoded by a nucleic acid sequence of SEQ ID NO: 71, 33, or 65, and a TCR-β comprising an amino acid sequence which is encoded by a nucleic acid sequence of SEQ ID NO: 73, 37, or, 69.

In some aspects, the recombinant T cell receptors (TCRs), or the antigen-binding fragment thereof, of the disclosure, comprise a TCR-α comprising an amino acid sequence which is encoded by a nucleic acid sequence of SEQ ID NO: 72, 34, or 66, and a TCR-β comprising an amino acid sequence which is encoded by a nucleic acid sequence of SEQ ID NO: 74, 38, or 70.

In some aspects, the recombinant TCR, or the antigen-binding fragment thereof, of the disclosure is humanized or chimeric.

The term “chimeric” TCR refers to TCRs wherein the amino acid sequence is derived from two or more species. Typically, the variable regions of the TCR correspond to the variable region of TCRs derived from one species of mammals (e.g. mouse, rat, rabbit, etc.) with the desired specificity, affinity, and capability while the constant regions are homologous to the sequences in TCRs derived from another (usually human) to avoid eliciting an immune response in that species. The term “humanized” TCR refers to forms of non-human (e.g. murine) TCRs that contain minimal non-human (e.g., murine) sequences. Typically, humanized TCRs are human TCRs in which residues from the complementarity determining regions (CDRs) are replaced by residues from the CDRs of a non-human species (e.g. mouse, rat, rabbit, and hamster) that have the desired specificity, affinity, and capability (“CDR grafted”. Examples of methods used to generate humanized TCRs are described in (Chen et al., A humanized TCR retaining authentic specificity and affinity conferred potent anti-tumour cytotoxicity, Immunology, 2018, 155(1): 123-136).

In some aspects, if the recombinant TCR, or the antigen-binding fragment thereof, contains a CD3 gamma, delta, or epsilon polypeptide, the cytoplasmic domain of the engineered TCR can include intracellular signaling domains; and TCR alpha and TCR beta subunits usually lack signaling domains. The intracellular signaling domain is generally responsible for activating at least one normal effector function of immune cells into which the TCR has been introduced.

In some aspects, the disclosure is directed to polynucleotides encoding one or more of the recombinant TCRs of the disclosure, or one or more of the antigen-binding fragments thereof. In some aspects, the polynucleotides encode the alpha chain (TCR-α) of the recombinant TCR, or of the antigen-binding fragment thereof, of the disclosure. In some aspects, the polynucleotides encode a CDR1α, a CDR2α, and a CDR3a. In some aspects, the polynucleotides encode a variable alpha chain (TCR-α). In some aspects, the polynucleotides encode a full length alpha chain (TCR-α). In some aspects, the polynucleotides encode beta chain (TCR-β) of the recombinant TCR, or of the antigen-binding fragment thereof, of the disclosure. In some aspects, the polynucleotides encode the alpha chain (TCR-α) and the beta chain (TCR-β) of the recombinant TCR, or of the antigen-binding fragment thereof, of the disclosure. In some aspects, the polynucleotides encode a CDR1β, a CDR2β, and a CDR3β. In some aspects, the polynucleotides encode a variable beta chain (TCR-β). In some aspects, the polynucleotides encode a full length beta chain (TCR-β).

In some aspects, the disclosure is directed to vectors (e.g., expression vectors) comprising the polynucleotides encoding the recombinant TCR, or the antigen-binding fragment thereof, of the disclosure. The polynucleotide comprised in such vectors may encode the alpha chain (TCR-α), or the beta chain (TCR-β), or both the alpha chain (TCR-α) and the beta chain (TCR-β) of the recombinant TCR, or of the antigen-binding fragment thereof, of the disclosure. In some aspects, the polynucleotides encode a CDR1α, a CDR2α, a CDR3α, CDR1β, a CDR2β, and a CDR3β. In some aspects, the polynucleotides encode a variable alpha chain (TCR-α) and a variable beta chain (TCR-β). In some aspects, the polynucleotides encode a full length alpha chain (TCR-α) and a full length beta chain (TCR-β). In some aspects, the polynucleotides comprised in such vectors may encode the variable alpha chain (TCR-α), or the variable beta chain (TCR-β), or both the variable alpha chain (TCR-α) and the variable beta chain (TCR-β). In some aspects, the polynucleotides comprised in such vectors may encode the full length alpha chain (TCR-α), or the full length beta chain (TCR-β), or both the full length alpha chain (TCR-α), or the full length beta chain (TCR-β).

The polynucleotides and the vectors of the disclosure may be obtained by any of techniques well-known in the art for the manipulation of nucleic acid sequences outside an organism. For example, the nucleotide sequences encoding the desired T cell receptors (TCRs), or antigen-binding fragments thereof, derived from tumor antigen-specific T cells of interest, may be isolated and cloned in a vector (e.g., expression vector) by any of the techniques well-known in the art (see for example Green & Sambrook Molecular Cloning: A Laboratory Manual, volumes 1-3, 4^{th} edition). In some embodiments, the recombinant T cell receptors (TCRs), or antigen-binding fragment thereof, are cloned from naturally occurring T cells. In some embodiments, the recombinant T cell receptors (TCRs), or antigen-binding fragment thereof, are artificially synthesized. In some embodiments, the recombinant TCR, or antigen-binding fragment thereof, comprises an a and a β chain (also known as TCRα and TCRβ, respectively) or a γ and a δ chain (also known as TCRγ and TCRδ, respectively), or antigen-binding fragments thereof. In some embodiments, the recombinant TCR, or antigen-binding fragment thereof, is in the αβ form. In some aspects, the recombinant TCR, or antigen-binding fragment thereof, is in the γδ form. In some embodiments, the recombinant TCR, or antigen-binding fragment thereof (e.g., antigen-binding fragment thereof), is capable of binding to an antigen peptide (i.e., epitope) bound to a MHC receptor (e.g., a human leucocyte antigen-A (HLA-A)*03:01 allele). In some embodiments, the epitope is derived from a Kirsten Rat Sarcoma Viral Oncogene Homolog (KRAS) protein comprising a G12V mutation. In some aspects, the epitope is presented on a human leucocyte antigen-A (HLA-A). In some embodiments, the epitope is presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the polynucleotides of the disclosure, encoding one or more of the recombinant TCRs, or of the antigen-binding fragments thereof, disclosed herein, are cloned into vectors including but not limited to plasmids, phagemids, phage derivatives, animal viruses, and cosmids. Viruses that can be used as vectors include, but are not limited to, retrovirus, adenovirus, adeno-associated virus, herpes virus, and lentivirus. The present invention is not limited to the use of constitutive promoters, while inducible promoters are also considered. The use of an inducible promoter provides a molecular switch that can initiate the expression of an operably linked polynucleotide sequence when expression is required, or close the expression when expression is not required. Examples of inducible promoters include, but are not limited to, NFAT6 promoter, metallothionein promoter, glucocorticoid promoter, progesterone promoter, and tetracycline-regulated promoter.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell.

In some aspects, the polynucleotides of the disclosure encode recombinant T cell receptors (TCRs), or antigen-binding fragments thereof, comprising an alpha chain (TCR-α) and/or a beta chain (TCR-β), or antigen-binding fragments thereof. In some aspects, the polynucleotide of the disclosure encodes a TCR-α comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64. In some aspects, the polynucleotide of the disclosure encodes a TCR-β comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68. In some aspects, the polynucleotide of the disclosure encodes a TCR-α comprising an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64, and a TCR-β comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68.

In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31 or 63. In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 17, 35, or 67. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 18, 36, or 68.

In some aspects, the polynucleotide of the disclosure encodes a TCR-α comprising an amino acid sequence of SEQ ID NO: 15, 16, 31, 32, 63, or 64. In some aspects, the polynucleotide of the disclosure encodes a TCR-β comprising an amino acid sequence of SEQ ID NO: 17, 18, 35, 36, 67, or 68. In some aspects, the polynucleotide of the disclosure encodes a TCR-α comprising an amino acid sequence of SEQ ID NO: 15, 16, 31, 32, 63, or 64, and a TCR-β comprising an amino acid sequence of SEQ ID NO: 17, 18, 35, 36, 67, or 68.

In some aspects, the TCR alpha chain comprises an amino acid sequence of SEQ ID NO: 15, 31 or 63. In some aspects, the TCR alpha chain comprises an amino acid sequence of SEQ ID NO: 16, 32, or 64. In some aspects, the TCR beta chain comprises an amino acid sequence of SEQ ID NO: 17, 35, or 67. In some aspects, the TCR beta chain comprises an amino acid sequence of SEQ ID NO: 18, 36, or 68. In some aspects, a polynucleotide of the disclosure encodes an alpha chain (TCR-α) comprising (i) a variable TCR alpha chain complementarity determining region 1 (CDR1α) of SEQ ID NO: 3, or 4, (ii) a variable TCR alpha chain complementarity determining region 2 (CDR2α) of SEQ ID NO: 5, or 6, (iii) a variable TCR alpha chain complementarity determining region 3 (CDR3α) of SEQ ID NO: 7, or 8. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively.

In some aspects, the polynucleotides of the disclosure encode a beta chain (TCR-β) comprising (i) a variable TCR beta chain complementarity determining region 1 (CDR1β) of SEQ ID NO: 9, or 10, (ii) a variable TCR beta chain complementarity determining region 2 (CDR2β) of SEQ ID NO: 11, or 12, (iii) variable TCR beta chain complementarity determining region 3 (CDR3 β) of SEQ ID NO: 13, or 14. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the polynucleotides of the disclosure encode an alpha chain (TCR-α) comprising (i) a variable TCR alpha chain complementarity determining region 1 (CDR1α) of SEQ ID NO: 3, or 4, (ii) a variable TCR alpha chain complementarity determining region 2 (CDR2α) of SEQ ID NO: 5, or 6, and (iii) a variable TCR alpha chain complementarity determining region 3 (CDR3α) of SEQ ID NO: 7, or 8; and (b) a beta chain (TCR-β) comprising (i) a variable TCR beta chain complementarity determining region 1 (CDR1β) of SEQ ID NO: 9, or 10, (ii) a variable TCR beta chain complementarity determining region 2 (CDR2β) of SEQ ID NO: 11, or 12, and (iii) variable TCR beta chain complementarity determining region 3 (CDR3 β) of SEQ ID NO: 13, or 14. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the polynucleotides of the disclosure encode recombinant T cell receptors (TCRs), or antigen-binding fragments thereof, comprising an alpha chain (TCR-α) and/or a beta chain (TCR-D), or antigen-binding fragments thereof. In some aspects, the polynucleotide of the disclosure encoding a TCR-α comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 72, 33, 34, 65, or 66. In some aspects, the polynucleotides of the disclosure encoding a TCR-β comprises a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 74, 37, 38, 69, or 70. In some aspects, the polynucleotide of the disclosure encoding a TCR-α and a TCR-β comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 72, 33, 34, 65, or 66, and a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 74, 37, 38, 69, or 70.

In some aspects, the polynucleotides of the disclosure encoding a TCR-α comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 33, or 65. In some aspects, the polynucleotide of the disclosure encoding a TCR-β comprises a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 37, or, 69. In some aspects, the polynucleotide of the disclosure encoding a TCR-α and a TCR-β comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 71, 33, or 65, and a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 73, 37, or, 69.

In some aspects, the polynucleotides of the disclosure encoding a TCR-α comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 72, 34, or 66. In some aspects, the polynucleotide of the disclosure encoding a TCR-β comprises a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 74, 38, or 70. In some aspects, the polynucleotide of the disclosure encoding a TCR-α and a TCR-β comprises a nucleic acid sequence having at least 85%, at least 90%, at least 91% at least 92%, at least 93%, at least 94%, at least 95%, at least 96%, at least 97%, at least 98%, or at least 99% sequence identity to SEQ ID NO: 72, 34, or 66, and a nucleic acid sequence having at least 85%, at least 90%, at least 91%, at least 92%, at least 93%, at least 94%, at least 95%, at least 96%, at least 97%, at least 98%, or at least 99% sequence identity to SEQ ID NO: 74, 38, or 70.

In some aspects, the polynucleotides of the disclosure encoding a TCR-α comprise a nucleic acid sequence of SEQ ID NO: 71, 72, 33, 34, 65, or 66. In some aspects, the polynucleotide of the disclosure encoding a TCR-β comprises a nucleic acid sequence of SEQ ID NO: 73, 74, 37, 38, 69, or 70. In some aspects, the polynucleotide of the disclosure encoding a TCR-α and a TCR-β comprises a nucleic acid sequence of SEQ ID NO: 71, 72, 33, 34, 65, or 66, and a nucleic acid sequence of SEQ ID NO: 73, 74, 37, 38, 69, or 70.

In some aspects, the polynucleotides of the disclosure encoding a TCR-α comprise a nucleic acid sequence of SEQ ID NO: 71, 33, or 65. In some aspects, the polynucleotide of the disclosure encoding a TCR-β comprises a nucleic acid sequence of SEQ ID NO: 73, 37, or, 69. In some aspects, the polynucleotide of the disclosure encoding a TCR-α and a TCR-β comprises a nucleic acid sequence of SEQ ID NO: 71, 33, or 65, and a nucleic acid sequence of SEQ ID NO: 73, 37, or, 69.

In some aspects, the polynucleotides of the disclosure encoding a TCR-α comprise a nucleic acid sequence of SEQ ID NO: 72, 34, or 66. In some aspects, the polynucleotide of the disclosure encoding a TCR-β comprises a nucleic acid sequence of SEQ ID NO: 74, 38, or 70. In some aspects, the polynucleotide of the disclosure encoding a TCR-α and a TCR-β comprises a nucleic acid sequence of SEQ ID NO: 72, 34, or 66, and a nucleic acid sequence of SEQ ID NO: 74, 38, or 70.

In some aspects, the polynucleotides of the disclosure comprise a nucleotide sequence of (i) SEQ ID NO: 19, or 20, (ii) SEQ ID NO: 21, or 22, and (iii) SEQ ID NO: 23 or 24. In some aspects, a polynucleotide of the disclosure comprises a nucleotide sequence of (i) SEQ ID NO: 19, (ii) SEQ ID NO: 21, and (iii) SEQ ID NO: 23. In some aspects, a polynucleotide of the disclosure comprises a nucleotide sequence of (i) SEQ ID NO: 20, (ii) SEQ ID NO: 22, and (iii) SEQ ID NO: 24.

In some aspects, the polynucleotides of the disclosure comprise a nucleotide sequence of (i) SEQ ID NO: 25, or 26, (ii) SEQ ID NO: 27, or 28, and (iii) SEQ ID NO: 29, or 30. In some aspects, a polynucleotides of the disclosure comprises a nucleotide sequence of (i) SEQ ID NO: 25, (ii) SEQ ID NO: 27, and (iii) SEQ ID NO: 29. In some aspects, a polynucleotides of the disclosure comprises a nucleotide sequence of (i) SEQ ID NO: 26, (ii) SEQ ID NO: 28, and (iii) SEQ ID NO: 30.

In some aspects, the polynucleotides of the disclosure comprise (i) a nucleotide sequence of SEQ ID NO: 19, or 20, encoding a variable TCR alpha chain complementarity determining region 1 (CDR1α), (ii) a nucleotide sequence of SEQ ID NO: 21, or 22, encoding a variable TCR alpha chain complementarity determining region 2 (CDR2α), (iii) a nucleotide sequence of SEQ ID NO: 23 or 24, encoding a variable TCR alpha chain complementarity determining region 3 (CDR3α), (iv) a nucleotide sequence of SEQ ID NO: 25, or 26, encoding a variable TCR beta chain complementarity determining region 1 (CDR1β), (v) a nucleotide sequence of SEQ ID NO: 27, or 28, encoding a variable TCR beta chain complementarity determining region 2 (CDR2β), and (vi) a nucleotide sequence of SEQ ID NO: 29, or 30, encoding a variable TCR beta chain complementarity determining region 3 (CDR3 β).

In some aspects, the polynucleotides of the disclosure comprise (i) a nucleotide sequence of SEQ ID NO: 19, encoding a variable TCR alpha chain complementarity determining region 1 (CDR1α), (ii) a nucleotide sequence of SEQ ID NO: 21, encoding a variable TCR alpha chain complementarity determining region 2 (CDR2α), (iii) a nucleotide sequence of SEQ ID NO: 23, encoding a variable TCR alpha chain complementarity determining region 3 (CDR3α), (iv) a nucleotide sequence of SEQ ID NO: 25, encoding a variable TCR beta chain complementarity determining region 1 (CDR1β), (v) a nucleotide sequence of SEQ ID NO: 27, encoding a variable TCR beta chain complementarity determining region 2 (CDR2β), and (vi) a nucleotide sequence of SEQ ID NO: 29, encoding a variable TCR beta chain complementarity determining region 3 (CDR3β).

In some aspects, the polynucleotides of the disclosure comprise (i) a nucleotide sequence of SEQ ID NO: 20, encoding a variable TCR alpha chain complementarity determining region 1 (CDR1α), (ii) a nucleotide sequence of SEQ ID NO: 22, encoding a variable TCR alpha chain complementarity determining region 2 (CDR2α), (iii) a nucleotide sequence of SEQ ID NO: 24, encoding a variable TCR alpha chain complementarity determining region 3 (CDR3α), (iv) a nucleotide sequence of SEQ ID NO: 26, encoding a variable TCR beta chain complementarity determining region 1 (CDR1β), (v) a nucleotide sequence of SEQ ID NO: 28, encoding a variable TCR beta chain complementarity determining region 2 (CDR2β), and (vi) a nucleotide sequence of SEQ ID NO: 30, encoding a variable TCR beta chain complementarity determining region 3 (CDR3 β).

In some aspects, the recombinant TCR of the disclosure, or antigen-binding fragments thereof, binds to an epitope of a Kirsten Rat Sarcoma Viral Oncogene Homolog (KRAS) protein comprising a G12V mutation. In some aspects, the recombinant TCR of the disclosure, or antigen-binding fragments thereof, binds to an epitope of a Kirsten Rat Sarcoma Viral Oncogene Homolog (KRAS) protein comprising a G12V mutation presented on a human leucocyte antigen-A (HLA-A). In some aspects, the recombinant TCR of the disclosure, or antigen-binding fragments thereof, binds to an epitope of a Kirsten Rat Sarcoma Viral Oncogene Homolog (KRAS) protein comprising a G12V mutation presented on a human leucocyte antigen-A (HLA-A)*03:01. In some aspects, Kirsten Rat Sarcoma Viral Oncogene Homolog (KRAS) protein comprising a G12V mutation is presented on a human leucocyte antigen-A (HLA-A) (e.g., HLA-A* 03:01) on a cell. In some aspects, Kirsten Rat Sarcoma Viral Oncogene Homolog (KRAS) protein comprising a G12V mutation is presented on a human leucocyte antigen-A (HLA-A)*03:01 on a cancer cell. In some aspects, Kirsten Rat Sarcoma Viral Oncogene Homolog (KRAS) protein comprising a G12V mutation is presented on a human leucocyte antigen-A (HLA-A) (e.g., HLA-A*03:01) on a lung cancer cell. In some aspects, Kirsten Rat Sarcoma Viral Oncogene Homolog (KRAS) protein comprising a G12V mutation is presented on a human leucocyte antigen-A (HLA-A) (e.g., HLA-A*03:01) on a non-small cell lung cancer (NSCLC) cancer cell. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the recombinant TCR, or antigen-binding fragments thereof, of the disclosure are expressed in a cancer cell. In some aspects, the cancer cell is derived from a cancer selected from the group consisting of: a lung cancer (e.g., a non-small cell lung cancer (NSCLC)), a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, and any combination thereof. In some aspects, the cancer is selected from the group consisting of: a lung cancer (e.g., a non-small cell lung cancer (NSCLC)), a colorectal cancer, a pancreatic cancer, or any combination thereof. In some aspects, the cancer is a non-small cell lung cancer (NSCLC). In some aspects, the cancer is a colorectal cancer. In some aspects, the cancer is a pancreatic cancer. In some aspects, the cancer is a tumor (e.g., a primary tumor or a metastatic tumor).

In some aspects, the recombinant TCR, or antigen-binding fragments thereof, of the disclosure are expressed in a cell. In some aspects, the recombinant TCR, or antigen-binding fragments thereof, of the disclosure are expressed in a host cell. In some aspects, the host cell is a T cell. In some aspects, the recombinant TCR, or the antigen-binding fragments thereof, of the disclosure, are expressed on the surface of the host cell (e.g., T cell). In some aspects, the recombinant TCR, or antigen-binding fragments thereof of the disclosure, direct the host cell (e.g., T cell) to a cancer cell. In some aspects, the cancer cell expresses a Kirsten Rat Sarcoma Viral Oncogene Homolog (KRAS) protein comprising a G12V mutation. In some aspects, the cancer cell expresses a Kirsten Rat Sarcoma Viral Oncogene Homolog (KRAS) protein comprising a G12V mutation comprising an epitope presented on a human leucocyte antigen-A (HLA-A). In some aspects, the cancer cell expresses a Kirsten Rat Sarcoma Viral Oncogene Homolog (KRAS) protein comprising a G12V mutation comprising an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the cancer cell is selected from the group consisting of: a lung cancer cell (e.g., a non-small cell lung cancer (NSCLC) cell), a colorectal cancer cell, a pancreatic cancer cell, an appendiceal cancer cell, a small bowel adenocarcinoma cell, an hepatobiliary cancer cell, a gynecological malignancy cell, a hematopoietic cancer cell, a breast cancer cell, a bladder cancer cell, a prostate cancer cell, a skin cancer cell, or any combination thereof. In some aspects, the cancer cell is selected from the group consisting of: a lung cancer cell (e.g., a non-small cell lung cancer (NSCLC) cell), a colorectal cancer cell, a pancreatic cancer cell, or any combination thereof.

In some aspects, provided herein is a method of treating cancer or a tumor in an subject, the method comprising administering to the subject a therapeutically effective amount of any of the recombinant T cell receptors (TCRs), or antigen-binding fragment thereof, the polynucleotides, the vectors, or the host cells (e.g., T cell) disclosed herein. In some aspects, the cancer comprises cancer cells expressing a Kirsten Rat Sarcoma Viral Oncogene Homolog (KRAS) protein comprising a G12V mutation. In some aspects, the cancer comprises cancer cells expressing a Kirsten Rat Sarcoma Viral Oncogene Homolog (KRAS) protein comprising a G12V mutation, which comprises an epitope presented on a human leucocyte antigen-A (HLA-A). In some aspects, the cancer comprises cancer cells expressing a Kirsten Rat Sarcoma Viral Oncogene Homolog (KRAS) protein comprising a G12V mutation, which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the subject is a human.

### Bispecific T Cell Engagers (BiTEs)

In some aspects, the disclosure is directed to a Bi-specific T-cell engager (BiTE) comprising the recombinant TCRs of the disclosure or antigen-binding fragment thereof.

In some aspects, the BiTE is capable of binding to an epitope of a Kirsten Rat Sarcoma Viral Oncogene Homolog (KRAS) protein comprising a G12V mutation. In some aspects, the BiTE is capable of binding to an epitope of a Kirsten Rat Sarcoma Viral Oncogene Homolog (KRAS) protein comprising a G12V mutation presented on a human leucocyte antigen-A (HLA-A). In some aspects, the BiTE is capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

A bi-specific antibody molecule as employed herein refers to a molecule with two antigen binding domains, which may bind the same or different antigens. A BiTE is a subclass of bispecific antibody molecules. “Non-IgG-like” antibodies include antibodies that lack an Fc portion such as bispecific T cell engagers (BiTE), DART, tetravalent antiparallel structures (TandAbs) and VH-only bi-nanobodies. The non-IgG-like antibodies due to their lack of an Fc portion are smaller and have shorter in vivo half-lives.

As used herein, the term “Bi-specific T-cell engagers”, or “BiTEs”, refer to a class of artificial bispecific monoclonal antibodies that are investigated for the use as anti-cancer drugs. They direct a host's immune system, more specifically the T cells' cytotoxic activity, against cancer cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Like other bispecific antibodies, and unlike ordinary monoclonal antibodies, BiTEs form a link between T cells and tumor cells. This causes T cells to exert cytotoxic activity on tumor cells by producing proteins like perforin and granzymes, independently of the presence of MHC I or co-stimulatory molecules. These proteins enter tumor cells and initiate the cell's apoptosis. This action mimics physiological processes observed during T cell attacks against tumor cells. BiTE is a registered trademark of Micromet AG (fully owned subsidiary of Amgen Inc).

BiTEs are able to form a link between T cells and tumor cells by virtue of their specificities for an antigen on the T cell and an antigen on the tumour cell. This leads to activation of the T-cells and triggers the T cells to exert their cytotoxic effects on tumour cells, independently of MHC I or co-stimulatory molecules.

In some aspects, the BiTe harbors specificity towards antibody or antigen fragments targets expressed on the surface of cells (e.g., cancer cells or targeted epitopes of interest) and exhibit specificity for CD3 coreceptor of a T cell in addition to the disclosed engineered or antigen binding fragment disclosed herein. In some aspects, the present disclosure BiTE is capable of activation of antigen specific T cells, which can kill targeted cancer cells expressing a particular epitopes of interest (e.g., a Kirsten Rat Sarcoma Viral Oncogene Homolog (KRAS) protein comprising a G12V mutation). In some aspects, the present disclosure BiTE is capable of activation of antigen specific T cells, which can kill targeted cancer cells expressing a particular epitopes of interest (e.g., a Kirsten Rat Sarcoma Viral Oncogene Homolog (KRAS) protein comprising a G12V mutation, which comprises an epitope presented on a human leucocyte antigen-A (HLA-A)). In some aspects, the present disclosure BiTE is capable of activation of antigen specific T cells, which can kill targeted cancer cells expressing a particular epitopes of interest (e.g., a Kirsten Rat Sarcoma Viral Oncogene Homolog (KRAS) protein comprising a G12V mutation, which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the BiTE is specific for at least a surface antigen on a T cell of interest. Examples of T cell surface antigens include but are not limited to: CD3, CD2, VLA-1, CD8, CD4, CCR6, CXCR5, CD25, CD31, CD45RO, CD197, CD127, CD38, CD27, CD196, CD277 and CXCR3, particularly CD2, CD3, CD31 and CD277.

In some aspects, the BiTE comprises (i) a binding region specific to a surface antigen on a T cell of interest (e.g., selected from CD3 (such as CD3 delta, CD3 epsilon or CD3 gamma), and (ii) an antigen binding region (e.g., specific to an antigen comprising a Kirsten Rat Sarcoma Viral Oncogene Homolog (KRAS) protein comprising a G12V mutation which comprises an epitope presented on a human leucocyte antigen-A (HLA-A)*03:01) comprising TCR-α chain and TCR-β chain disclosed herein. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the immune cell engager (such as T cell engager) is arranged is the format VL1-linker1-VH1-linker1-VH2-linker3-VL2.

In some aspects, the BiTEs disclosed herein bind to an epitope of a Kirsten Rat Sarcoma Viral Oncogene Homolog (KRAS) protein comprising a G12V mutation. In some aspects, the BiTEs disclosed herein bind to an epitope of a Kirsten Rat Sarcoma Viral Oncogene Homolog (KRAS) protein comprising a G12V mutation presented on a human leucocyte antigen-A (HLA-A). In some aspects, the BiTEs disclosed herein bind to an epitope of a Kirsten Rat Sarcoma Viral Oncogene Homolog (KRAS) protein comprising a G12V mutation presented on a human leucocyte antigen-A (HLA-A)*03:01. In some aspects, the epitope of the Kirsten Rat Sarcoma Viral Oncogene Homolog (KRAS) protein comprising a G12V mutation is presented on a human leucocyte antigen-A (HLA-A) (e.g., HLA-A *03:01) on a cell. In some aspects, the epitope of the Kirsten Rat Sarcoma Viral Oncogene Homolog (KRAS) protein comprising a G12V mutation is presented on a human leucocyte antigen-A (HLA-A)*03:01 on a cancer cell. In some aspects, the epitope of the Kirsten Rat Sarcoma Viral Oncogene Homolog (KRAS) protein comprising a G12V mutation is presented on a human leucocyte antigen-A (HLA-A) (e.g., HLA-A *03:01) on a lung cancer cell. In some aspects, the epitope of the Kirsten Rat Sarcoma Viral Oncogene Homolog (KRAS) protein comprising a G12V mutation is presented on a human leucocyte antigen-A (HLA-A) (e.g., HLA-A *03:01) on a non-small cell lung cancer (NSCLC) cancer cell. In some aspects, the epitope comprises the amino acid sequence of VVGAVGVGK (SEQ ID NO: 1). In some aspects, the epitope comprises the amino acid sequence of VVVGAVGVGK (SEQ ID NO: 2) In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the cancer cell is selected from the group consisting of: a lung cancer cell, a non-small cell lung cancer (NSCLC) cell, a colorectal cancer cell, a pancreatic cancer cell, an appendiceal cancer cell, a small bowel adenocarcinoma cell, an hepatobiliary cancer cell, a gynecological malignancy cell, a hematopoietic cancer cell, a breast cancer cell, a bladder cancer cell, a prostate cancer cell, a skin cancer cell, or any combination thereof. In some aspects, the cancer cell is selected from the group consisting of: a lung cancer cell, a non-small cell lung cancer (NSCLC) cell, a colorectal cancer cell, a pancreatic cancer cell, or any combination thereof.

In some aspects, the BiTEs disclosed herein are expressed in a cell. In some aspects, the BiTEs disclosed herein are expressed in a host cell. In some aspects, the host cell is a T cell. In some aspects, the recombinant TCR of the disclosure, or antigen-binding domains thereof, are expressed on the surface of the host cell (e.g., T cell). In some aspects, the BiTEs disclosed herein direct the host cell (e.g., T cell) to a cancer cell. In some aspects, the cancer cell expresses a Kirsten Rat Sarcoma Viral Oncogene Homolog (KRAS) protein comprising a G12V mutation. In some aspects, the cancer cell expresses a Kirsten Rat Sarcoma Viral Oncogene Homolog (KRAS) protein comprising a G12V mutation which comprises an epitope presented on a human leucocyte antigen-A (HLA-A). In some aspects, the cancer cell expresses a Kirsten Rat Sarcoma Viral Oncogene Homolog (KRAS) protein comprising a G12V mutation which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

Some aspects of the disclosure are directed to a polynucleotide encoding a BiTE disclosed herein. In some aspects, the polynucleotide of the disclosure encodes a BiTE comprising a TCR of the disclosure.

In some aspects, provided herein is a method of treating cancer or a tumor in an subject, the method comprising administering to the individual a therapeutically effective amount of any of the of the BiTE disclosed herein. In some aspects, the subject is a human. In some aspects, the subject is a human. In some aspects, the cancer comprises cancer cells expressing a Kirsten Rat Sarcoma Viral Oncogene Homolog (KRAS) protein comprising a G12V mutation. In some aspects, the subject is a human. In some aspects, the cancer comprises cancer cells expressing a Kirsten Rat Sarcoma Viral Oncogene Homolog (KRAS) protein comprising a G12V mutation, which comprises an epitope presented on a human leucocyte antigen-A (HLA-A). In some aspects, the cancer comprises cancer cells expressing a Kirsten Rat Sarcoma Viral Oncogene Homolog (KRAS) protein comprising a G12V mutation, which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the subject is a human.

### TCR T Cells

Certain aspects of the disclosure are directed to engineered cells comprising a recombinant T cell receptor (TCR), or an antigen-binding fragment thereof, of the disclosure, a polynucleotide encoding such a TCR, and/or an expression vector comprising such a polynucleotide. In some aspects, the cell is a T cell. In some aspects, the T cell is a CD4+ cell, a CD8+ cell, or a CD4+/CD8+ cell. In some aspects, the host cell is a T cell which is a CD4+ cell, a CD8+ cell, or a CD4+/CD8+ cell.

In some aspects, the cell is a modified cell. As used herein, the term “modified cell” can refer to any type of cell (e.g., a primary cell, a cell in culture, or a cell from a cell line). For example, a modified cell can be a cell which is manipulated in vitro, by any technique known in the art, to achieve expression of a specific protein (e.g., a cell exposed to an APC presenting a specific antigen to achieve expression of a specific TCR). In some aspects, the modified cell can be a cell which is transformed, transfected, or transduced with a nucleic acid molecule (e.g., and expression vector), and the progeny or potential progeny of such a cell. A modified cell transformed, transfected, or transduced with a nucleic acid molecule can be a host cell, a recombinant cell, or an engineered cell. For example an engineered cell can include a cell which has been transformed, transfected or transduced with an expression vector comprising a nucleic acid sequence encoding a specific TCR. The progeny of such a cell may not be identical to the parent cell transfected with the nucleic acid molecule, e.g., due to mutations or environmental influences that may occur in succeeding generations or integration of the nucleic acid molecule into the host cell genome.

As used herein, the term “transfected” or “transformed” or “transduced” refers to the process by which exogenous nucleic acid is transferred or introduced into a host cell. A “transfected” or “transformed” or “transduced” cell is a cell that has been transfected, transformed or transduced with exogenous nucleic acid. The cells include primary cell of a subject and progenies thereof. As used herein, the term “transfection” refers to the process of introducing nucleic acids into cells by non-viral methods. As used herein, the term “transduction” refers to the process whereby foreign DNA is introduced into another cell via a viral vector.

In some aspects, the host cells (e.g., T cells) of the disclosure are modified to comprise a recombinant TCR, or antigen-binding fragment thereof, of the disclosure. In some aspects, the recombinant T cell receptor (TCR), or antigen-binding fragment thereof, is capable of binding to an epitope of a Kirsten Rat Sarcoma Viral Oncogene Homolog (KRAS) protein comprising a G12V mutation. In some aspects, the recombinant T cell receptor (TCR), or antigen-binding fragment thereof, is capable of binding to an epitope of a Kirsten Rat Sarcoma Viral Oncogene Homolog (KRAS) protein comprising a G12V mutation presented on a human leucocyte antigen-A (HLA-A). In some aspects, the recombinant T cell receptor (TCR), or antigen-binding fragment thereof, is capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the recombinant T cell receptor (TCR), or antigen-binding fragment thereof, is expressed on the surface of the host cell (e.g., engineered T cell). In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the TCR is a recombinant TCR.

In some aspects, before expansion and genetic modification, a source of T cells is obtained from a subject. Examples of subjects include mammals such as humans, primates, dogs, cats, mice, rats, and transgenic species thereof. T cells can be obtained from many sources, including peripheral blood mononuclear cells, bone marrow, lymph node tissue, umbilical cord blood, thymus tissue, tissue from the site of infection, ascites, pleural effusion, spleen tissue, and tumors. In certain aspects of the disclosure, any number of T cell lines available in the art can be used. In some aspects, any number of techniques known to a skilled person, such as Ficoll separation technology, can be used to obtain T cells from blood units collected from a subject. In some aspects, cells from the circulating blood of an individual are obtained by apheresis. Products obtained by apheresis blood apheresis usually contain lymphocytes, including T cells, monocytes, granulocytes, B cells, other nucleated white blood cells, red blood cells and platelets. In some aspects, the cells collected by apheresis can be washed to remove the plasma fraction and placed in an appropriate buffer or medium for subsequent processing steps. Multiple rounds of selection can also be used in the context of the invention. In some aspects, it may be necessary to perform a selection procedure and use “unselected” cells during activation and expansion. “Unselected” cells can also undergo other rounds of selection.

In some aspects, the T cell of the present disclosure can be expanded by contacting with a surface to which an agent that stimulates the CD3/TCR complex-related signal and a ligand that stimulates a costimulatory molecule on the surface of the T cell are attached. In particular, the T cell population can be stimulated, for example by contacting with an anti-CD3 antibody or antigen-binding fragments thereof or an anti-CD2 antibody immobilized on a surface, or by contacting a protein kinase C activator (for example, bryostatin) and calcium ionophore.

CD3 (Cluster of Differentiation 3) T cell co-receptor is a protein complex composed of four different chains. In mammals, the complex contains one CD3 gamma chain, CD3 delta chain, and two CD3 epsilon chains. These chains have a molecule of accessory T cell receptor (TCR) and zeta-chain to generate activation signals for T lymphocytes. The TCR, zeta chain and CD3 molecule together constitute a T cell receptor complex. The CD3 molecule is connected to the T cell receptor (TCR) through a salt bridge to form a TCR-CD3 complex, which participates in the signaling of T cells, and is mainly used to label thymocytes, T lymphocytes and T cell lymphomas. The cytoplasmic segment of CD3 contains immunoreceptor tyrosine-based activation motif (ITAM). TCR recognizes and binds to the antigen peptide presented by the MHC (major histo-compatibility complex) molecule, resulting in the tyrosine residues in the conserved sequence of ITAM of CD3. Being phosphorylated by the tyrosine protein kinase p56lck in T cells, and then recruiting other tyrosine protein kinases (such as ZAP-70) containing SH2 (Scr homology 2) domain. The phosphorylation of ITAM and the binding to ZAP-70 are one of the important biochemical reactions in the early stages of the signaling process of T cell activation. Therefore, the function of the CD3 molecule is to transduce the activation signal generated by the TCR to recognize the antigen. In this application, the exogenous receptor that can bind to the target antigen and trigger CD3 signal activation includes at least one CD3 binding site and at least one additional antigen binding site specific to bacterial substance, viral protein, autoimmune marker, or antigen present specific cells (e.g., cell surface proteins of B cells, T cells, natural killer (NK) cells, bone marrow cells, phagocytes, or tumor cells). Such exogenous receptors can cross-link two kinds of cells and can be used to direct T cells to specific targets and trigger the cytotoxic activity of T cells on the target cells. Examples of such targets may be tumor cells or infectious agents, such as viral pathogens or bacterial pathogens.

Prior to in vitro manipulation or genetic modification of the immune effector cells disclosed herein, the source of cells can be obtained from a subject. In particular, the immune effector cells for use with the TCRs as disclosed herein comprise T cells. In some aspects, the T cells are obtained from peripheral blood mononuclear cells, bone marrow, lymph nodes tissue, cord blood, thymus issue, tissue from a site of an infection, ascites, pleural effusion, spleen tissue, or a tumor. In some aspects, the T cells are obtained from peripheral blood mononuclear cells. In some aspects, the T cells are obtained from tumor cells (e.g., NSCLC cells). In some aspects, T cells can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis can be washed to remove the plasma fraction and to place the cells in an appropriate buffer or media for subsequent processing. In some aspects, the cells are washed with PBS. In some aspects, the washed solution lacks calcium, and can lack magnesium or can lack many, if not all, divalent cations. As would be appreciated by those of ordinary skill in the art, a washing step can be accomplished by methods known to those in the art, such as by using a semiautomated flowthrough centrifuge. After washing, the cells can be resuspended in a variety of biocompatible buffers or other saline solution with or without buffer. In some aspects, the undesirable components of the apheresis sample can be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting can also be used to isolate cell populations of interest for use in the present disclosure.

PBMCs can be used directly for genetic modification with the various methods as disclosed herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells. In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+T helper cells can be sorted into naive, central memory, and effector cells by identifying cell populations that have cell surface antigens. CD4+ lymphocytes can be obtained by standard methods.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the T cell receptors disclosed herein (e.g., transduced with a viral vector comprising a nucleic acid encoding an a and 8 chain) and then are activated and expanded in vitro. Methods for activating and expanding T cells are known in the art and are disclosed, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells can be activated and stimulated to proliferate with feeder cells and appropriate antibodies and cytokines using methods such as those disclosed in U.S. Pat. Nos. 6,040,177; 5,827,642; and WO2012129514.

In some aspects, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The cells can be autologous or heterologous to the patient undergoing therapy. If desired, the treatment can also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) as disclosed herein to enhance induction of the immune response.

Humoral immune responses, mediated primarily by helper T cells capable of activating B cells thus leading to antibody production, may be induced. A variety of techniques may be used for analyzing the type of immune responses induced by the compositions of the present disclosure, which are well disclosed in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

In some aspects, the immune cell is a T cell. In some aspects, the immune cell is a T lymphocyte selected from an inflammatory T lymphocyte, a cytotoxic T lymphocyte, a regulatory T lymphocyte, or a helper T lymphocyte. In some cases, the immune cell is a CD8+ cytotoxic T lymphocyte.

Whether prior to or after genetic modification of the engineered cells (e.g., T cells), even if the genetically modified immune cells of the present disclosure are activated and proliferate independently of antigen binding mechanisms, the immune cells, particularly T-cells of the present disclosure can be further activated and expanded generally using methods as disclosed, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

In some aspects, the T cells of the disclosure are expanded by contact with an agent that stimulates a CD3 TCR complex and a costimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations can be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that can contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFp, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% O2). T cells that have been exposed to varied stimulation times can exhibit different characteristics.

In some aspects, the cells can be expanded by co-culturing with tissue or cells. The cells can also be expanded in vivo, for example in the subject's blood after administrating said cell into the subject.

In some aspects, the modified cells (e.g., engineered T cells) of the disclosure comprise a recombinant T cell receptor (TCR), or antigen-binding fragment thereof, capable of binding to an epitope of a Kirsten Rat Sarcoma Viral Oncogene Homolog (KRAS) protein comprising a G12V mutation. In some aspects, the modified cells (e.g., engineered T cells) of the disclosure comprise a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A). In some aspects, the modified cells (e.g., engineered T cells) of the disclosure comprise a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the modified cells (e.g., engineered T cells) of the disclosure comprise a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation. In some aspects, the modified cells (e.g., engineered T cells) of the disclosure comprise a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A). In some aspects, the modified cells (e.g., engineered T cells) of the disclosure comprise a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the modified cells (e.g., engineered T cells) of the disclosure comprise a vector comprising a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation. In some aspects, the modified cells (e.g., engineered T cells) of the disclosure comprise a vector comprising a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A). In some aspects, the modified cells (e.g., engineered T cells) of the disclosure comprise a vector comprising a polynucleotide encoding a recombinant T cell receptor (TCR), or antigen-binding fragment thereof, capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the modified cells (e.g., engineered T cells) comprise (α) a recombinant T cell receptor (TCR), or antigen-binding fragment thereof, and/or (b) a polynucleotide encoding a recombinant T cell receptor (TCR), or antigen-binding fragment thereof or a vector comprising a polynucleotide encoding a recombinant T cell receptor (TCR) or antigen-binding fragment thereof, wherein the recombinant the T-cell Receptor (TCR) comprises: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 634 and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68, wherein the engineered TCR binds an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence VVGAVGVGK (SEQ ID NO: 1). In some aspects, the epitope comprises the amino acid sequence VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the TCR alpha chain comprises CDR1α, CDR2α, and CDR3α of SEQ ID NO: 15, 31 or 63. In some aspects, the TCR alpha chain comprises CDR1α, CDR2α, and CDR3α of SEQ ID NO: 16, 32, or 64. In some aspects, the TCR beta chain comprises CDR1β, CDR2β, and CDR3β of SEQ ID NO: 17, 35, or 67. In some aspects, the TCR beta chain comprises CDR1β, CDR2β, and CDR3β of SEQ ID NO: 18, 36, or 68.

In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, and/or (c) a vector comprising a polynucleotide encoding a recombinant T cell receptor (TCR) or antigen-binding fragment thereof, wherein the recombinant T cell receptor is capable of binding to an epitope of a Kirsten Rat Sarcoma Viral Oncogene Homolog (KRAS) protein comprising a G12V mutation. In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, and/or (c) a vector comprising a polynucleotide encoding a recombinant T cell receptor (TCR) or antigen-binding fragment thereof, wherein the recombinant T cell receptor is capable of binding to an epitope of a Kirsten Rat Sarcoma Viral Oncogene Homolog (KRAS) protein comprising a G12V mutation presented on a human leucocyte antigen-A (HLA-A). In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, and/or (c) a vector comprising a polynucleotide encoding a recombinant T cell receptor (TCR) or antigen-binding fragment thereof, wherein the recombinant T cell receptor is capable of binding to an epitope of a Kirsten Rat Sarcoma Viral Oncogene Homolog (KRAS) protein comprising a G12V mutation presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the TCR comprises: (a) a TCR-alpha chain comprising (i) a variable TCR alpha chain complementarity determining region 1 (CDR1α) having at least 85%, at least 90%, at least 91%, at least 92%, at least 93%, at least 94%, at least 95%, at least 96%, at least 97%, at least 98%, at least 99%, sequence identity to SEQ ID NO: 3, or 4, (ii) a variable TCR alpha chain complementarity determining region 2 (CDR2α) having at least 85%, at least 90%, at least 91%, at least 92%, at least 93%, at least 94%, at least 95%, at least 96%, at least 97%, at least 98%, at least 99%, sequence identity to SEQ ID NO: 5, or 6, (iii) a variable TCR alpha chain complementarity determining region 3 (CDR3α) having at least 85%, at least 90%, at least 91%, at least 92%, at least 93%, at least 94%, at least 95%, at least 96%, at least 97%, at least 98%, at least 99%, sequence identity to SEQ ID NO: 7, or 8, and (b) a TCR beta chain comprising (iv) a variable TCR beta chain complementarity determining region 1 (CDR1β) having at least 85%, at least 90%, at least 91%, at least 92%, at least 93%, at least 94%, at least 95%, at least 96%, at least 97%, at least 98%, at least 99%, sequence identity to SEQ ID NO: 9, or 10, (v) a variable TCR beta chain complementarity determining region 2 (CDR2β) having at least 85%, at least 90%, at least 91%, at least 92%, at least 93%, at least 94%, at least 95%, at least 96%, at least 97%, at least 98%, at least 99%, sequence identity to SEQ ID NO: 11, or 12, (vi) variable TCR beta chain complementarity determining region 3 (CDR3β) having at least 85%, at least 90%, at least 91%, at least 92%, at least 93%, at least 94%, at least 95%, at least 96%, at least 97%, at least 98%, at least 99%, sequence identity to SEQ ID NO: 13, or 14.

In some aspects, (i) the CDR1α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 3, (ii) the CDR2α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 5, (iii) the CDR3α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 7, (iv) the CDR1β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 9, (v) the CDR2β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 11 (vi) the CDR3β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 13.

In some aspects, (i) the CDR1α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 4, (ii) the CDR2α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 6, (iii) the CDR3α comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 8, (iv) the CDR1β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 10, (v) the CDR2β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 12, (vi) the CDR3β comprises an amino acid sequence having at least 85%, at least 90%, at least 91%, at least 92%, at least 93%, at least 94%, at least 95%, at least 96%, at least 97%, at least 98%, at least 99%, sequence identity to SEQ ID NO: 14.

In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, and/or (c) a vector comprising a polynucleotide encoding a recombinant T cell receptor (TCR) or antigen-binding fragment thereof, wherein the recombinant T cell receptor (TCR) comprises (i) a variable TCR alpha chain complementarity determining region 1 (CDR1α) comprising the amino acid sequence of SEQ ID NO: 3, or 4, (ii) a variable TCR alpha chain complementarity determining region 2 (CDR2α) comprising the amino acid sequence of SEQ ID NO: 5, or 6, (iii) a variable TCR alpha chain complementarity determining region 3 (CDR3α) comprising the amino acid sequence of SEQ ID NO: 7, or 8, (iv) a variable TCR beta chain complementarity determining region 1 (CDR1β) comprising the amino acid sequence of SEQ ID NO: 9, or 10, (v) a variable TCR beta chain complementarity determining region 2 (CDR2β) comprising the amino acid sequence of SEQ ID NO: 11, or 12, and (vi) variable TCR beta chain complementarity determining region 3 (CDR3β) comprising the amino acid sequence of SEQ ID NO: 13, or 14. In some aspects, the CDR1α, CDR2α, and CDR3α comprise or consist of SEQ ID NOs: 3, 5, and 7, respectively. In some aspects, the CDR1α, CDR2α, and CDR3α comprise or consist of SEQ ID NOs: 4, 6, and 8, respectively. In some aspects, the CDR1β, CDR2β, and CDR3β comprise or consist of SEQ ID NOs: 9, 11, and 13, respectively. In some aspects, the CDR1β, CDR2β, and CDR3β comprise or consist of SEQ ID NOs: 10, 12, and 14, respectively.

In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, or a vector comprising a polynucleotide encoding a recombinant T cell receptor (TCR) or antigen-binding fragment thereof, wherein the recombinant T cell receptor (TCR), or antigen-binding fragment thereof, comprises an alpha chain (TCR-α) and a beta chain (TCR-β), and wherein the TCR-α comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64, and the TCR-β comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68.

In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31 or 63. In some aspects, the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 17, 35, or 67. In some aspects, the TCR beta chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 18, 36, or 68.

In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, or a vector comprising a polynucleotide encoding a recombinant T cell receptor (TCR) or antigen-binding fragment thereof, wherein the recombinant T cell receptor (TCR), or antigen-binding fragment thereof, comprises an alpha chain (TCR-α) of SEQ ID NO: 15, 16, 31, 32, 63, or 64, and a beta chain (TCR-β) of SEQ ID NO: 17, 18, 35, 36, 67, or 68.

In some aspects, the TCR alpha chain comprises an amino acid sequence of SEQ ID NO: 15, 31 or 63. In some aspects, the TCR alpha chain comprises an amino acid sequence of SEQ ID NO: 16, 32, or 64. In some aspects, the TCR beta chain comprises an amino acid sequence of SEQ ID NO: 17, 35, or 67. In some aspects, the TCR beta chain comprises an amino acid sequence of SEQ ID NO: 18, 36, or 68.

In some aspects, the modified cells (e.g., engineered T cells) comprise (a) a recombinant T cell receptor (TCR), or antigen-binding fragment thereof, and/or (b) a polynucleotide encoding a recombinant T cell receptor (TCR), or antigen-binding fragment thereof, or a vector comprising a polynucleotide encoding a recombinant T cell receptor (TCR) or antigen-binding fragment thereof, wherein the recombinant T cell receptor (TCR) is humanized or chimeric.

In some aspects, the modified cell (e.g., engineered T cells) is capable of recognition of a cell expressing a Kirsten Rat Sarcoma Viral Oncogene Homolog (KRAS) protein comprising a G12V mutation. In some aspects, the modified cell (e.g., engineered T cells) is capable of recognition of a cell expressing a Kirsten Rat Sarcoma Viral Oncogene Homolog (KRAS) protein comprising a G12V mutation, which comprises an epitope presented on a human leucocyte antigen-A (HLA-A). In some aspects, the modified cell (e.g., engineered T cells) is capable of recognition of a cell expressing a Kirsten Rat Sarcoma Viral Oncogene Homolog (KRAS) protein comprising a G12V mutation, which comprises an epitope presented on a human leucocyte antigen-A (HLA-A)*03:01. In some aspects, the recognition of the cell expressing a Kirsten Rat Sarcoma Viral Oncogene Homolog (KRAS) protein comprising a G12V mutation, which comprises an epitope presented on a human leucocyte antigen-A (HLA-A)*03:01 comprises a human leucocyte antigen-A (HLA-A)*03:01-restricted binding of the TCR to an epitope of the Kirsten Rat Sarcoma Viral Oncogene Homolog (KRAS) protein comprising a G12V mutation expressed by the cancer cell. In some aspects the cell expressing the Kirsten Rat Sarcoma Viral Oncogene Homolog (KRAS) protein comprising a G12V mutation, which comprises an epitope presented on a human leucocyte antigen-A (HLA-A)*03:01 is a cancer cell. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the cancer cell is selected from the group consisting of: a lung cancer cell, a non-small cell lung cancer (NSCLC) cell, a colorectal cancer cell, a pancreatic cancer cell, an appendiceal cancer cell, a small bowel adenocarcinoma cell, an hepatobiliary cancer cell, a gynecological malignancy cell, a hematopoietic cancer cell, a breast cancer cell, a bladder cancer cell, a prostate cancer cell, a skin cancer cell, and any combination thereof. In some aspects, the cancer cell is selected from the group consisting of: a lung cancer cell, a non-small cell lung cancer (NSCLC) cell, a colorectal cancer cell, a pancreatic cancer cell, and any combination thereof.

In some aspects, provided herein is a method of treating cancer or a tumor in a subject, the method comprising administering to the individual a therapeutically effective amount of any of the modified T cells disclosed herein. In some aspects, the subject is a human. In some aspects, the cancer comprises cancer cells expressing a Kirsten Rat Sarcoma Viral Oncogene Homolog (KRAS) protein comprising a G12V mutation. In some aspects, the subject is a human. In some aspects, the cancer comprises cancer cells expressing a Kirsten Rat Sarcoma Viral Oncogene Homolog (KRAS) protein comprising a G12V mutation which comprises an epitope presented on a human leucocyte antigen-A (HLA-A). In some aspects, the subject is a human. In some aspects, the cancer comprises cancer cells expressing a Kirsten Rat Sarcoma Viral Oncogene Homolog (KRAS) protein comprising a G12V mutation which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the cancer is selected from the group consisting of: a lung cancer (e.g., a non-small cell lung cancer (NSCLC)), a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, and any combination thereof. In some aspects, the cancer is selected from the group consisting of: a lung cancer (e.g., a non-small cell lung cancer (NSCLC)), a colorectal cancer, a pancreatic cancer, and any combination thereof.

In some aspects, the subject is a human.

### Methods of Treatment/Use

Certain aspects of the disclosure are directed to methods of killing a target cancer cell using the recombinant TCRs, polynucleotide, vectors, host cells (e.g. TCR T-cells), or BiTE molecules disclosed herein. In some aspects, the disclosure provides methods of killing a target cancer cell in subjects harboring G12V mutation on Kirsten Rat Sarcoma Viral Oncogene Homolog (KRAS) protein. In some aspects, the disclosure provides methods of killing a target cancer cell in subjects harboring G12V mutation on Kirsten Rat Sarcoma Viral Oncogene Homolog (KRAS) protein which comprises an epitope presented on a human leucocyte antigen-A (HLA-A). In particular, the disclosure provides methods of killing a target cancer cell in subjects harboring G12V mutation on Kirsten Rat Sarcoma Viral Oncogene Homolog (KRAS) protein which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

Certain aspects of the disclosure are directed to methods prophylaxis and/or therapy of a subject diagnosed with, suspected of having or at risk for developing or recurrence of a cancer, comprising administering to the subject any of the TCRs, polynucleotide, vectors, host cells (e.g. TCR T-cells), or BiTE molecules disclosed herein. In some aspects, the disclosure provides TCR based therapies for subject harboring G12V mutation on Kirsten Rat Sarcoma Viral Oncogene Homolog (KRAS) protein. In some aspects, the disclosure provides TCR based therapies for subject harboring G12V mutation on Kirsten Rat Sarcoma Viral Oncogene Homolog (KRAS) protein which comprises and epitope presented on a human leucocyte antigen-A (HLA-A). In particular, the disclosure provides TCR based therapies for subject harboring G12V mutation on Kirsten Rat Sarcoma Viral Oncogene Homolog (KRAS) protein which comprises and epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, the cancer is selected from the group consisting of: a lung cancer, a non-small cell lung cancer (NSCLC), a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, or any combination thereof. In some aspects, the cancer is selected from the group consisting of: a lung cancer, a non-small cell lung cancer (NSCLC), a colorectal cancer, a pancreatic cancer, or any combination thereof.

In some aspects, the subject is a human.

Cytotoxic T lymphocytes (CTLs) that recognize mutated peptides (neoantigens) presented at the tumor cell surface by HLA class I molecules represent powerful antitumor immune cells that are capable of causing regressions of large tumors in cancer patients. Recombinant T-cell receptors (TCRs) derived from such tumor antigen-specific T cells as disclosed herein can be cloned and isolated to create TCR-based therapeutics with anti-cancer targeting ability, including engineered TCR-T cells or BiTE therapeutics.

As a natural consequence of high HLA diversity and the private nature of most tumor-associated mutations, very few neoantigen targets are shared between patients, and neoantigen-specific TCRs are very few in number and at very early stages of development in clinical trials. The therapies herein can be directed to engineered T-cell receptors that target the shared Kirsten Rat Sarcoma Viral Oncogene Homolog (KRAS) protein G12V mutation, which is expresses by 25% of NSCLC patients, and is presented by the most prevalent HLA-A*03:01 allele. Given the high prevalence of KRAS mutations in lung cancer (e.g., NSCLC) and the high prevalence of this HLA molecules in the human population, the engineered TCR based therapies disclosed herein can have therapeutic value for many lung cancer patients that present a G12V mutation on the Kirsten Rat Sarcoma Viral Oncogene Homolog (KRAS) protein, e.g., lung cancer patients that present a G12V mutation on the Kirsten Rat Sarcoma Viral Oncogene Homolog (KRAS) protein, which comprises an epitope presented on a HLA-A*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2).

In some aspects, provided herein is method for prophylaxis and/or therapy of an individual diagnosed with, suspected of having or at risk for developing or recurrence of a cancer, comprising administering to the individual any of the recombinant molecules or modified cells disclosed herein (e.g., the recombinant T cell receptors (TCRs), or antigen-binding fragment thereof, the polynucleotides, the vectors, or the host cells (e.g., TCR-T cell)).

In some aspects, the cancer comprises cancer cells expressing a Kirsten Rat Sarcoma Viral Oncogene Homolog (KRAS) protein comprising a G12V mutation. In some aspects, the cancer comprises cancer cells expressing a Kirsten Rat Sarcoma Viral Oncogene Homolog (KRAS) protein comprising a G12V mutation which comprises an epitope presented on a human leucocyte antigen-A (HLA-A). In some aspects, the cancer comprises cancer cells expressing a Kirsten Rat Sarcoma Viral Oncogene Homolog (KRAS) protein comprising a G12V mutation which comprises an epitope presented on a human leucocyte antigen-A (HLA-A)*03:01. In some aspects, the epitope comprises the amino acid sequence of VVGAVGVGK (SEQ ID NO: 1). In some aspects, the epitope comprises the amino acid sequence of VVVGAVGVGK (SEQ ID NO: 2). In some aspects, the epitope consists of the amino acid sequence of VVGAVGVGK (SEQ ID NO: 1). In some aspects, the epitope consists of the amino acid sequence of VVVGAVGVGK (SEQ ID NO: 2). In some aspects, the cancer is selected from the group consisting of: a lung cancer, a non-small cell lung cancer (NSCLC), a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, or any combination thereof. In some aspects, the cancer is selected from the group consisting of: a lung cancer, a non-small cell lung cancer (NSCLC), a colorectal cancer, a pancreatic cancer, or any combination thereof.

In some aspects, the method comprises administering a therapeutically effective dose of the recombinant T cell receptors (TCRs), or antigen-binding fragment thereof, the polynucleotides, the vectors, or the host cells (e.g., modified cells, e.g., TCR-T cell) to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the bispecific antibody is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In certain aspects, the bispecific antibody is administered intratumorally.

The amount of the recombinant T cell receptors (TCRs), or of the antigen-binding fragment thereof, or of the polynucleotides, or of the vectors, or of the host cells (e.g., modified cells, e.g., TCR-T cell), which will be effective in the treatment or prevention of a condition will depend on the nature of the disease. The precise dose to be employed in a composition will also depend on the route of administration, and the seriousness of the disease.

In some aspects, the subject is a human.

### EXAMPLES

### Example 1. Analysis of Antigen-Specific T Cells from Healthy Donor Blood

Normal donor's peripheral blood mononuclear cells (PBMCs) were cultured with Dendritic Cell (DC) growth factor granulocyte-macrophage colony-stimulating factor (GM-CSF) and IL-4 for one week to get autologous dendritic cells.

After treatment with Tumor Necrosis Factor alpha (TNF-α), IL-6 and IL-1b for 24 hours, the matured DC were pulsed with G12V peptide. The peptide pulsed DC together with IL-21 were subsequently co-cultured with the PBMC from the same donor, at a ratio 1:35 of DC to PBMC, for one week to stimulate antigen specific T cell growth. The same procedure was then repeated twice.

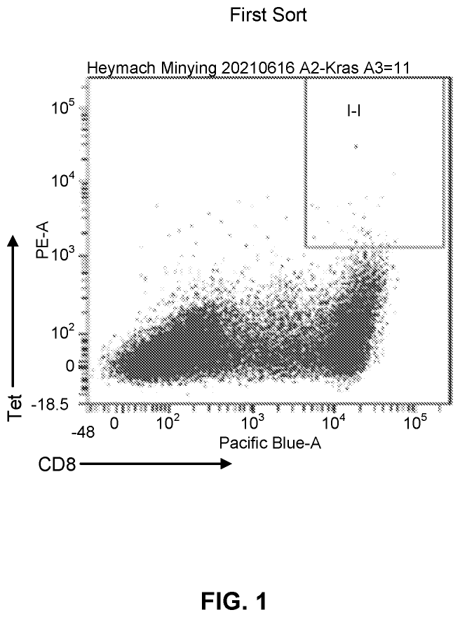

After 2 weeks of co-culture, T cells were collected and stained with PE-labeled G12V tetramer and a CD8 antibody. The Tetramer/CD8 double positive T cells were then sorted by flowcytometry after a first stimulation (FIG. 1)

Following DC stimulation and antigen specific T cell sorting, the number of G12V tetramer/CD8 double positive T cell consistently resulted in low volume.

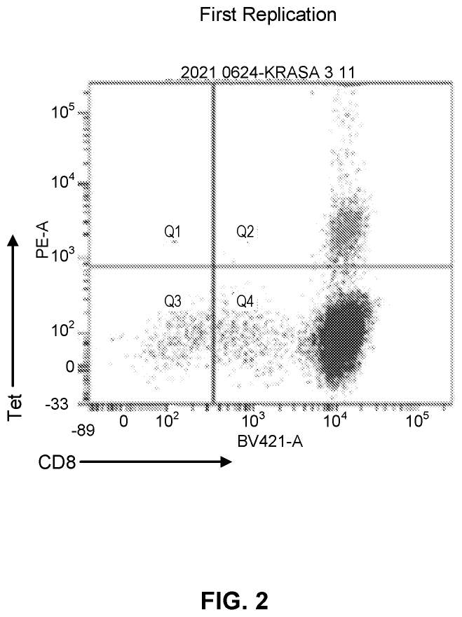

To achieve a suitable level of T cells for a functional assay function and TCR identification, double positive T cells from the first stimulation and sorting were collected and utilized in rapid T cell replication procedure by stimulation with anti-CD3 (OKT) antibody and IL-2 for two weeks (FIG. 2).

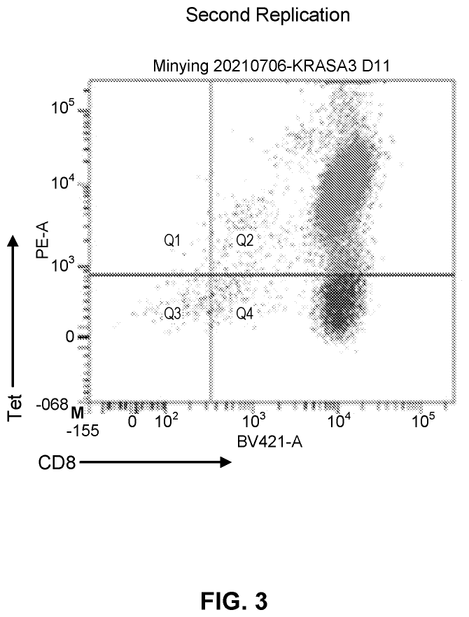

The T cells were then stained and sorted again for second replication and used in killing and TCR α/β determination, as shown in FIG. 3.

### Example 2. TCR Transduction into Donor PBMCs

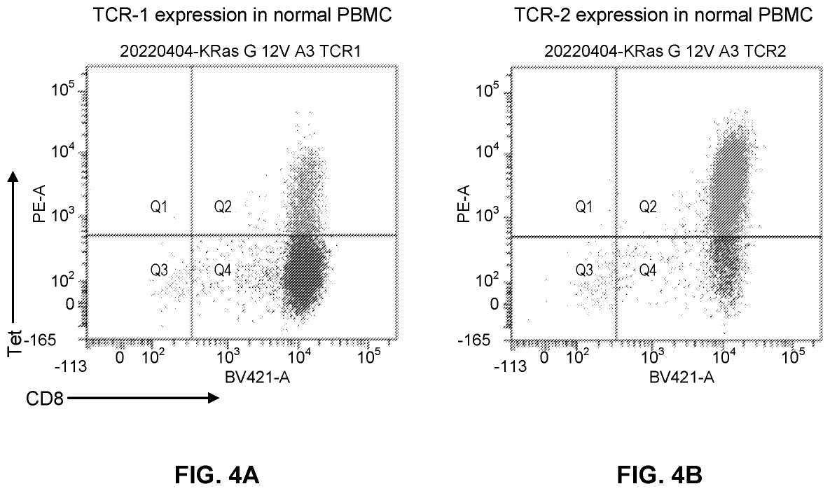

TCR α/β chains were then cloned into a retroviral vector pMSGV1 and stably transduced into normal donor's PBMC (FIG. 4A-B).

Following a 5 day transduction period, the G12V tetramer/CD8 double positive T cells were then sorted out for rapid T cell replication. Sorting of the G12V TCR expression T cells after rapid T cell replication then took place.

### Example 3. Analysis of the Differences in Killing Ability of T Cells

To analyze G12V specific T cell activity, the Human lung cancer cell line NCI-1650 and NCI-H2444 (Dr. A. Gazdar) which carry KRAS G12V mutation, were used to determine TCR-1 killing activity. Human Caucasian lung small cell carcinoma, large cell variant SHP-77 (NCI-SHTP77 as described in Edwin R. et al., Cancer Res 38:3830, (1978)), which exhibits the endogenous G12V mutation, was stably transduced with HLAA0301 and used as a target for cytotoxicity killing assay assessment for TCR-2.

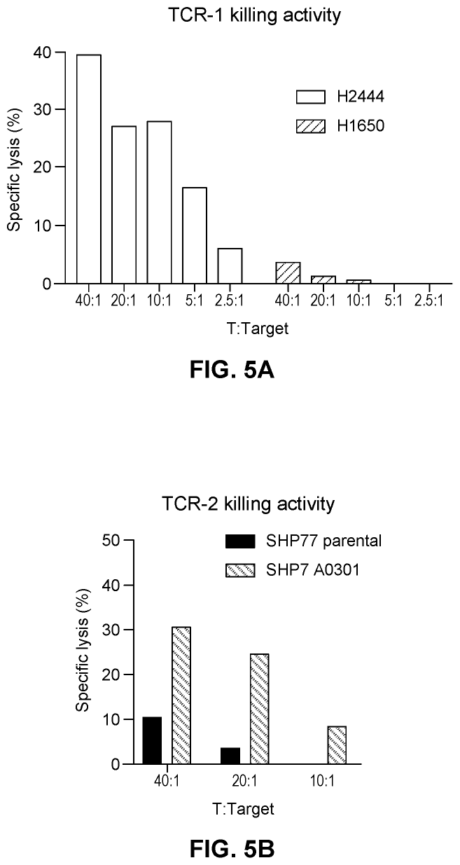

The SHP77 parental cell, HLAA0301 expressing SHP77 cell (SHP77A03), H1650 and H2444 cell were then appropriately labeled with chromium Cr51 for two hours, and co-cultured with G12V specific TCR-T cells at different effectors to target ratios for a total duration of 4 hours. The released Cr51 was then measured by a gamma counter and calculated for killing efficacy as shown in FIG. 5A-B.

### Example 4. Isolated TCR Sequences

TCRs in G12V specific T cell population were determined by single T cell RNA sequencing using 10× Genomics Chromium Service at the MID Anderson DNA core facility. The TCR α/β pair sequence (for two TCR α/β pair versions) was analyzed using the Cell Ranger program and sequenced. The full length V region (corresponding to SEQ ID NOs. 33, 34, 37 and 38) was sequenced. A partial sequence for the C region (corresponding to SEQ ID NOs. 41, 42, 45, and 46) was also obtained.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties. Any examples provided herein are offered by way of illustration and not by way of limitation.

TABLE 1
Sequences

| SEQ ID NO. | DESCRIPTION | SEQUENCE |
|---|---|---|
| SEQ ID NO. 1 | KRAS G12V peptide | VVGAVGVGK |
| | bound by the HLA- | |
| | A*03:01 allele (9-mer) | |
| SEQ ID NO. 2 | KRAS G12V peptide | VVVGAVGVGK |
| | bound by the HLA- | |
| | A*03:01 allele (10-mer) | |
| SEQ ID NO. 3 | TCR A CDR 1-1 | TSGFNG |
| SEQ ID NO. 4 | TCR A CDR 1-2 | NSMFDY |
| SEQ ID NO. 5 | TCR A CDR 2-1 | NVLDGL |
| SEQ ID NO. 6 | TCR A CDR 2-2 | ISSIKDK |
| SEQ ID NO. 7 | TCR A CDR 3-1 | AVKGRNSGYALN |
| SEQ ID NO. 8 | TCR A CDR 3-2 | AASERGRDDKII |
| SEQ ID NO. 9 | TCR B CDR 1-1 | SGDLS |
| SEQ ID NO. 10 | TCR B CDR 1-2 | SEHNR |
| SEQ ID NO. 11 | TCR B CDR 2-1 | YYNGEE |
| SEQ ID NO. 12 | TCR B CDR 2-2 | FQNEAQ |
| SEQ ID NO. 13 | TCR B CDR 3-1 | ASTIQGAAQETQY |
| SEQ ID NO. 14 | TCR B CDR 3-2 | ASSFSRGPETQY |
| SEQ ID NO. 15 | ALPHA chain C/V (not | QNIDQPTEMTATEGAIVQINCTYQTSGFNGLFWYQQHA |
| | including signal | GEAPTFLSYNVLDGLEEKGRFSSFLSRSKGYSYLLLKELQMK |
| | peptide)-1 | DSASYLCAVKGRNSGYALNFGKGTSLLVTPHIQNPDPAVY |
| | | QLRDSKSSDKSVCLFTDFDSQTNVSQSKDSDVYITDKCVLD |
| | | MRSMDFKSNSAVAWSNKSDFACANAFNNSIIPEDTFFPS |
| | | PESSCDVKLVEKSFETDTNLNFQNLSVIGFRILLLKVAGFNL |
| | | LMTLRLWSS |
| SEQ ID NO. 16 | ALPHA chain C/V (not | QQKNDDQQVKQNSPSLSVQEGRISILNCDYTNSMFDYFL |
| | including signal | WYKKYPAEGPTFLISISSIKDKNEDGRFTVFLNKSAKHLSLHI |
| | peptide)-2 | VPSQPGDSAVYFCAASERGRDDKIIFGKGTRLHILPNIQNP |
| | | DPAVYQLRDSKSSDKSVCLFTDFDSQTNVSQSKDSDVYIT |
| | | DKCVLDMRSMDFKSNSAVAWSNKSDFACANAFNNSIIPE |
| | | DTFFPSPESSCDVKLVEKSFETDTNLNFQNLSVIGFRILLLKV |
| | | AGFNLLMTLRLWSS |
| SEQ ID NO. 17 | Beta Chain C/V (not | GVTQTPKHLITATGQRVTLRCSPRSGDLSVYWYQQSLDQ |
| | including signal | GLQFLIQYYNGEERAKGNILERFSAQQFPDLHSELNLSSLEL |
| | peptide)-1 | GDSALYFCASTIQGAAQETQYFGPGTRLLVLEDLKNVFPPE |
| | | VAVFEPSEAEISHTQKATLVCLATGFYPDHVELSWWVNGK |
| | | EVHSGVCTDPQPLKEQPALNDSRYCLSSRLRVSATFWQN |
| | | PRNHFRCQVQFYGLSENDEWTQDRAKPVTQIVSAEAWG |
| | | RADCGFTSESYQQGVLSATILYEILLGKATLYAVLVSALVLM |
| | | AMVKRKDSRG |
| SEQ ID NO. 18 | Beta Chain C/V (not | GVSQDPRHKITKRGQNVTFRCDPISEHNRLYWYRQTLGQ |
| | including signal | GPEFLTYFQNEAQLEKSRLLSDRFSAERPKGSFSTLEIQRTE |
| | peptide)-2 | QGDSAMYLCASSFSRGPETQYFGPGTRLLVLEDLKNVFPP |
| | | EVAVFEPSEAEISHTQKATLVCLATGFYPDHVELSWWVNG |
| | | KEVHSGVCTDPQPLKEQPALNDSRYCLSSRLRVSATFWQ |
| | | NPRNHFRCQVQFYGLSENDEWTQDRAKPVTQIVSAEAW |
| | | GRADCGFTSESYQQGVLSATILYEILLGKATLYAVLVSALVL |
| | | MAMVKRKDSRG |
| SEQ ID NO. 19 | TCR A CDR 1-1 | ACATCTGGGTTCAACGGG |
| SEQ ID NO. 20 | TCR A CDR 1-2 | AACAGCATGTTTGATTAT |
| SEQ ID NO. 21 | TCR A CDR 2-1 | AATGTTCTGGATGGTTTG |
| SEQ ID NO. 22 | TCR A CDR 2-2 | ATAAGTTCCATTAAGGATAAA |
| SEQ ID NO. 23 | TCR A CDR 3-1 | GCTGTGAAGGGTAGGAATTCCGGGTATGCACTCAAC |
| SEQ ID NO. 24 | TCR A CDR 3-2 | GCAGCAAGCGAGCGAGGCAGAGATGACAAGATCATC |
| SEQ ID NO. 25 | TCR B CDR 1-1 | TCTGGAGACCTCTCT |
| SEQ ID NO. 26 | TCR B CDR 1-2 | TCTGAACACAACCGC |
| SEQ ID NO. 27 | TCR B CDR 2-1 | TATTATAATGGAGAAGAG |
| SEQ ID NO. 28 | TCR B CDR 2-2 | TTCCAGAATGAAGCTCAA |
| SEQ ID NO. 29 | TCR B CDR 3-1 | GCCAGCACTATACAGGGGGGGGCTCAAGAGACCCAGT |

AC

| | | |
|---|---|---|
| SEQ ID NO. 30 | TCR B CDR 3-2 | GCCAGCAGCTTTTCCAGGGGGCCAGAGACCCAGTAC |
| SEQ ID NO. 31 | TCR A Variable-1 | QNIDQPTEMTATEGAIVQINCTYQTSGFNGLFWYQQHA |
| | | GEAPTFLSYNVLDGLEEKGRFSSFLSRSKGYSYLLLKELQMK |
| | | DSASYLCAVKGRNSGYALNFGKGTSLLVTPH |
| SEQ ID NO. 32 | TCR A Variable-2 | QQKNDDQQVKQNSPSLSVQEGRISILNCDYTNSMFDYFL |
| | | WYKKYPAEGPTFLISISSIKDKNEDGRFTVFLNKSAKHLSLHI |
| | | VPSQPGDSAVYFCAASERGRDDKIIFGKGTRLHILPN |
| SEQ ID NO. 33 | TCR A Variable-1 | CAAAACATTGACCAGCCCACTGAGATGACAGCTACGGA |
| | | AGGTGCCATTGTCCAGATCAACTGCACGTACCAGACAT |
| | | CTGGGTTCAACGGGCTGTTCTGGTACCAGCAACATGCT |
| | | GGCGAAGCACCCACATTTCTGTCTTACAATGTTCTGGAT |
| | | GGTTTGGAGGAGAAAGGTCGTTTTTCTTCATTCCTTAGT |
| | | CGGTCTAAAGGGTACAGTTACCTCCTTTTGAAGGAGCTC |
| | | CAGATGAAAGACTCTGCCTCTTACCTCTGTGCTGTGAAG |
| | | GGTAGGAATTCCGGGTATGCACTCAACTTCGGCAAAGG |
| | | CACCTCGCTGTTGGTCACACCCCAT |
| SEQ ID NO. 34 | TCR A Variable-2 | CAACAGAAGAATGATGACCAGCAAGTTAAGCAAAATTC |
| | | ACCATCCCTGAGCGTCCAGGAAGGAAGAATTTCTATTCT |
| | | GAACTGTGACTATACTAACAGCATGTTTGATTATTTCCT |
| | | ATGGTACAAAAAATACCCTGCTGAAGGTCCTACATTCCT |
| | | GATATCTATAAGTTCCATTAAGGATAAAAATGAAGATG |
| | | GAAGATTCACTGTCTTCTTAAACAAAAGTGCCAAGCACC |
| | | TCTCTCTGCACATTGTGCCCTCCCAGCCTGGAGACTCTG |
| | | CAGTGTACTTCTGTGCAGCAAGCGAGCGAGGCAGAGAT |
| | | GACAAGATCATCTTTGGAAAAGGGACACGACTTCATAT |
| | | TCTCCCCAAT |
| SEQ ID NO. 35 | TCR B Variable-1 | GVTQTPKHLITATGQRVTLRCSPRSGDLSVYWYQQSLDQ |
| | | GLQFLIQYYNGEERAKGNILERFSAQQFPDLHSELNLSSLEL |
| | | GDSALYFCASTIQGAAQETQYFGPGTRLLVLE |
| SEQ ID NO. 36 | TCR B Variable-2 | GVSQDPRHKITKRGQNVTFRCDPISEHNRLYWYRQTLGQ |
| | | GPEFLTYFQNEAQLEKSRLLSDRFSAERPKGSFSTLEIQRTE |
| | | QGDSAMYLCASSFSRGPETQYFGPGTRLLVLE |
| SEQ ID NO. 37 | TCR B Variable-1 | GGAGTCACACAAACCCCAAAGCACCTGATCACAGCAAC |
| | | TGGACAGCGAGTGACGCTGAGATGCTCCCCTAGGTCTG |
| | | GAGACCTCTCTGTGTACTGGTACCAACAGAGCCTGGAC |
| | | CAGGGCCTCCAGTTCCTCATTCAGTATTATAATGGAGAA |
| | | GAGAGAGCAAAAGGAAACATTCTTGAACGATTCTCCGC |
| | | ACAACAGTTCCCTGACTTGCACTCTGAACTAAACCTGAG |
| | | CTCTCTGGAGCTGGGGGACTCAGCTTTGTATTTCTGTGC |
| | | CAGCACTATACAGGGGGGGGCTCAAGAGACCCAGTACT |
| | | TCGGGCCAGGCACGCGGCTCCTGGTGCTCGAG |
| SEQ ID NO. 38 | TCR B Variable-2 | GGAGTCTCCCAGGACCCCAGACACAAGATCACAAAGAG |
| | | GGGACAGAATGTAACTTTCAGGTGTGATCCAATTTCTG |
| | | AACACAACCGCCTTTATTGGTACCGACAGACCCTGGGG |
| | | CAGGGCCCAGAGTTTCTGACTTACTTCCAGAATGAAGCT |
| | | CAACTAGAAAAATCAAGGCTGCTCAGTGATCGGTTCTCT |
| | | GCAGAGAGGCCTAAGGGATCTTTCTCCACCTTGGAGAT |
| | | CCAGCGCACAGAGCAGGGGGACTCGGCCATGTATCTCT |
| | | GTGCCAGCAGCTTTTCCAGGGGGCCAGAGACCCAGTAC |
| | | TTCGGGCCAGGCACGCGGCTCCTGGTGCTCGAG |
| SEQ ID NO. 39 | Alpha chain constant | IQNPDPAVYQLRDSKSSDKSVCLFTDFDSQTNVSQSKDSD |
| | region-1 | VYITDKCVLDMRSMDFKSNSAVAWSNKSDFACANAFNN |
| | | SIIPEDTFFPSPESSCDVKLVEKSFETDTNLNFQNLSVIGFRIL |
| | | LLKVAGFNLLMTLRLWSS |
| SEQ ID NO. 40 | Alpha chain constant | IQNPDPAVYQLRDSKSSDKSVCLFTDFDSQTNVSQSKDSD |
| | region-2 | VYITDKCVLDMRSMDFKSNSAVAWSNKSDFACANAFNN |
| | | SIIPEDTFFPSPESSCDVKLVEKSFETDTNLNFQNLSVIGFRIL |
| | | LLKVAGFNLLMTLRLWSS |
| SEQ ID NO. 41 | Alpha chain constant | ATCCAGAACCCTGACCCTGCCGTGTACCAGCTGAGAGA |
| | region-1 | CTCTAAATCCAGTGACAAGTCTGTCTGCCTATTCACCGA |
| | | TTTTGATTCTCAAACAAATGTGTCACAAAGTAAGGATTC |
| | | TGATGTGTATATCACAGACAAATGCGTGCTAGACATGA |
| | | GGTCTATGGACTTCAAGAGCAACAGTGCTGTGGCCTGG |
| | | AGCAACAAATCTGACTTTGCATGTGCAAACGCCTTCAAC |
| | | AACAGCATTATTCCAGAAGACACCTTCTTCCCCAGCCCA |
| | | GAAAGTTCCTGTGATGTCAAGCTGGTCGAGAAAAGCTT |
| | | TGAAACAGATACGAACCTAAACTTTCAAAACCTGTCAGT |
| | | GATTGGGTTCCGAATCCTCCTCCTGAAAGTGGCCGGGT |
| | | TTAATCTGCTCATGACGCTGCGGCTGTGGTCCAGCTGA |
| SEQ ID NO. 42 | Alpha chain constant | ATCCAGAACCCTGACCCTGCCGTGTACCAGCTGAGAGA |
| | region-2 | CTCTAAATCCAGTGACAAGTCTGTCTGCCTATTCACCGA |
| | | TTTTGATTCTCAAACAAATGTGTCACAAAGTAAGGATTC |
| | | TGATGTGTATATCACAGACAAATGCGTGCTAGACATGA |
| | | GGTCTATGGACTTCAAGAGCAACAGTGCTGTGGCCTGG |
| | | AGCAACAAATCTGACTTTGCATGTGCAAACGCCTTCAAC |
| | | AACAGCATTATTCCAGAAGACACCTTCTTCCCCAGCCCA |
| | | GAAAGTTCCTGTGATGTCAAGCTGGTCGAGAAAAGCTT |
| | | TGAAACAGATACGAACCTAAACTTTCAAAACCTGTCAGT |
| | | GATTGGGTTCCGAATCCTCCTCCTGAAAGTGGCCGGGT |
| | | TTAATCTGCTCATGACGCTGCGGCTGTGGTCCAGCTGA |
| SEQ ID NO. 43 | Beta Chain constant | DLKNVFPPEVAVFEPSEAEISHTQKATLVCLATGFYPDHVE |
| | region-1 | LSWWVNGKEVHSGVCTDPQPLKEQPALNDSRYCLSSRLR |
| | | VSATFWQNPRNHFRCQVQFYGLSENDEWTQDRAKPVT |
| | | QIVSAEAWGRADCGFTSESYQQGVLSATILYEILLGKATLY |
| | | AVLVSALVLMAMVKRKDSRG |
| SEQ ID NO. 44 | Beta Chain constant | DLKNVFPPEVAVFEPSEAEISHTQKATLVCLATGFYPDHVE |
| | region-2 | LSWWVNGKEVHSGVCTDPQPLKEQPALNDSRYCLSSRLR |
| | | VSATFWQNPRNHFRCQVQFYGLSENDEWTQDRAKPVT |
| | | QIVSAEAWGRADCGFTSESYQQGVLSATILYEILLGKATLY |
| | | AVLVSALVLMAMVKRKDSRG |
| SEQ ID NO. 45 | Beta Chain constant | GACCTGAAAAACGTGTTCCCACCCGAGGTCGCTGTGTTT |
| | region-1 | GAGCCATCAGAAGCAGAGATCTCCCACACCCAAAAGGC |
| | | CACACTGGTGTGCCTGGCCACAGGCTTCTACCCCGACCA |
| | | CGTGGAGCTGAGCTGGTGGGTGAATGGGAAGGAGGTG |
| | | CACAGTGGGGTCTGCACAGACCCGCAGCCCCTCAAGGA |
| | | GCAGCCCGCCCTCAATGACTCCAGATACTGCCTGAGCA |
| | | GCCGCCTGAGGGTCTCGGCCACCTTCTGGCAGAACCCC |
| | | CGCAACCACTTCCGCTGTCAAGTCCAGTTCTACGGGCTC |
| | | TCGGAGAATGACGAGTGGACCCAGGATAGGGCCAAAC |
| | | CTGTCACCCAGATCGTCAGCGCCGAGGCCTGGGGTAGA |
| | | GCAGACTGTGGCTTCACCTCCGAGTCTTACCAGCAAGG |
| | | GGTCCTGTCTGCCACCATCCTCTATGAGATCTTGCTAGG |
| | | GAAGGCCACCTTGTATGCCGTGCTGGTCAGTGCCCTCG |
| | | TGCTGATGGCCATGGTCAAGAGAAAGGATTCCAGAGG |
| | | C |
| SEQ ID NO. 46 | Beta Chain constant | GACCTGAAAAACGTGTTCCCACCCGAGGTCGCTGTGTTT |
| | region-2 | GAGCCATCAGAAGCAGAGATCTCCCACACCCAAAAGGC |
| | | CACACTGGTGTGCCTGGCCACAGGCTTCTACCCCGACCA |
| | | CGTGGAGCTGAGCTGGTGGGTGAATGGGAAGGAGGTG |
| | | CACAGTGGGGTCTGCACAGACCCGCAGCCCCTCAAGGA |
| | | GCAGCCCGCCCTCAATGACTCCAGATACTGCCTGAGCA |
| | | GCCGCCTGAGGGTCTCGGCCACCTTCTGGCAGAACCCC |
| | | CGCAACCACTTCCGCTGTCAAGTCCAGTTCTACGGGCTC |
| | | TCGGAGAATGACGAGTGGACCCAGGATAGGGCCAAAC |
| | | CTGTCACCCAGATCGTCAGCGCCGAGGCCTGGGGTAGA |
| | | GCAGACTGTGGCTTCACCTCCGAGTCTTACCAGCAAGG |
| | | GGTCCTGTCTGCCACCATCCTCTATGAGATCTTGCTAGG |
| | | GAAGGCCACCTTGTATGCCGTGCTGGTCAGTGCCCTCG |
| | | TGCTGATGGCCATGGTCAAGAGAAAGGATTCCAGAGG |
| | | C |
| SEQ ID NO. 47 | Linker-1 | RAKRSGSGATNFSLLKQAGDVEENPGP |
| SEQ ID NO. 48 | Linker-2 | RAKRSGSGATNFSLLKQAGDVEENPGP |
| SEQ ID NO. 49 | Linker-1 | CGT GCC AAG CGA AGC GGA TCT GGC GCC ACG AAC |
| | | TTC TCT CTG TTA AAG CAA GCA GGA GAT GTT GAA |
| | | GAA AAC CCC GGG CCT |
| SEQ ID NO. 50 | Linker-2 | CGT GCC AAG CGA AGC GGA TCT GGC GCC ACG AAC |
| | | TTC TCT CTG TTA AAG CAA GCA GGA GAT GTT GAA |
| | | GAA AAC CCC GGG CCT |
| SEQ ID NO. 51 | Signal peptide alpha-1 | MWGVFLLYVSMKMGGTTG |
| SEQ ID NO. 52 | Signal peptide alpha-2 | MAMLLGASVLILWLQPDWVNS |
| SEQ ID NO. 53 | Signal peptide alpha-1 | ATGTGGGGAGTTTTCCTTCTTTATGTTTCCATGAAGATG |
| | | GGAGGCACTACAGGA |
| SEQ ID NO. 54 | Signal peptide alpha-2 | ATGGCCATGCTCCTGGGGGCATCAGTGCTGATTCTGTG |
| | | GCTTCAGCCAGACTGGGTAAACAGT |
| SEQ ID NO. 55 | Signal peptide beta-1 | MGFRLLCCVAFCLLGAGPVDS |
| SEQ ID NO. 56 | Signal peptide beta-2 | MGTSLLCWMALCLLGADHADT |
| SEQ ID NO. 57 | Signal peptide beta-1 | ATGGGCTTCAGGCTCCTCTGCTGTGTGGCCTTTTGTCTC |
| | | CTGGGAGCAGGCCCAGTGGATTCT |
| SEQ ID NO. 58 | Signal peptide beta-2 | ATGGGCACCAGCCTCCTCTGCTGGATGGCCCTGTGTCTC |
| | | CTGGGGGCAGATCACGCAGATACT |
| SEQ ID NO. 59 | full TCR sequence-1 | MGFRLLCCVAFCLLGAGPVDSGVTQTPKHLITATGQRVTL |
| | | RCSPRSGDLSVYWYQQSLDQGLQFLIQYYNGEERAKGNIL |
| | | ERFSAQQFPDLHSELNLSSLELGDSALYFCASTIQGAAQET |
| | | QYFGPGTRLLVLEDLKNVFPPEVAVFEPSEAEISHTQKATL |
| | | VCLATGFYPDHVELSWWVNGKEVHSGVCTDPQPLKEQP |
| | | ALNDSRYCLSSRLRVSATFWQNPRNHFRCQVQFYGLSEN |
| | | DEWTQDRAKPVTQIVSAEAWGRADCGFTSESYQQGVLS |
| | | ATILYEILLGKATLYAVLVSALVLMAMVKRKDSRGRAKRSG |
| | | SGATNFSLLKQAGDVEENPGPMWGVFLLYVSMKMGGTT |
| | | GQNIDQPTEMTATEGAIVQINCTYQTSGFNGLFWYQQH |
| | | AGEAPTFLSYNVLDGLEEKGRFSSFLSRSKGYSYLLLKELQM |
| | | KDSASYLCAVKGRNSGYALNFGKGTSLLVTPHIQNPDPAV |
| | | YQLRDSKSSDKSVCLFTDFDSQTNVSQSKDSDVYITDKCVL |
| | | DMRSMDFKSNSAVAWSNKSDFACANAFNNSIIPEDTFFP |
| | | SPESSCDVKLVEKSFETDTNLNFQNLSVIGFRILLLKVAGFN |
| | | LLMTLRLWSS |
| SEQ ID NO. 60 | full TCR sequence-2 | MGTSLLCWMALCLLGADHADTGVSQDPRHKITKRGQNV |
| | | TFRCDPISEHNRLYWYRQTLGQGPEFLTYFQNEAQLEKSR |
| | | LLSDRFSAERPKGSFSTLEIQRTEQGDSAMYLCASSFSRGP |
| | | ETQYFGPGTRLLVLEDLKNVFPPEVAVFEPSEAEISHTQKA |
| | | TLVCLATGFYPDHVELSWWVNGKEVHSGVCTDPQPLKEQ |
| | | PALNDSRYCLSSRLRVSATFWQNPRNHFRCQVQFYGLSE |
| | | NDEWTQDRAKPVTQIVSAEAWGRADCGFTSESYQQGVL |
| | | SATILYEILLGKATLYAVLVSALVLMAMVKRKDSRGRAKRS |
| | | GSGATNFSLLKQAGDVEENPGPMAMLLGASVLILWLQPD |
| | | WVNSQQKNDDQQVKQNSPSLSVQEGRISILNCDYTNSM |
| | | FDYFLWYKKYPAEGPTFLISISSIKDKNEDGRFTVFLNKSAK |
| | | HLSLHIVPSQPGDSAVYFCAASERGRDDKIIFGKGTRLHILP |
| | | NIQNPDPAVYQLRDSKSSDKSVCLFTDFDSQTNVSQSKDS |
| | | DVYITDKCVLDMRSMDFKSNSAVAWSNKSDFACANAFN |
| | | NSIIPEDTFFPSPESSCDVKLVEKSFETDTNLNFQNLSVIGFR |
| | | ILLLKVAGFNLLMTLRLWSS |
| SEQ ID NO. 61 | full TCR sequence-1 | ATGGGCTTCAGGCTCCTCTGCTGTGTGGCCTTTTGTCTC |
| | | CTGGGAGCAGGCCCAGTGGATTCTGGAGTCACACAAAC |
| | | CCCAAAGCACCTGATCACAGCAACTGGACAGCGAGTGA |
| | | CGCTGAGATGCTCCCCTAGGTCTGGAGACCTCTCTGTGT |
| | | ACTGGTACCAACAGAGCCTGGACCAGGGCCTCCAGTTC |
| | | CTCATTCAGTATTATAATGGAGAAGAGAGAGCAAAAGG |
| | | AAACATTCTTGAACGATTCTCCGCACAACAGTTCCCTGA |
| | | CTTGCACTCTGAACTAAACCTGAGCTCTCTGGAGCTGGG |
| | | GGACTCAGCTTTGTATTTCTGTGCCAGCACTATACAGGG |
| | | GGCGGCTCAAGAGACCCAGTACTTCGGGCCAGGCACG |
| | | CGGCTCCTGGTGCTCGAGGACCTGAAAAACGTGTTCCC |
| | | ACCCGAGGTCGCTGTGTTTGAGCCATCAGAAGCAGAGA |
| | | TCTCCCACACCCAAAAGGCCACACTGGTGTGCCTGGCC |
| | | ACAGGCTTCTACCCCGACCACGTGGAGCTGAGCTGGTG |
| | | GGTGAATGGGAAGGAGGTGCACAGTGGGGTCTGCACA |
| | | GACCCGCAGCCCCTCAAGGAGCAGCCCGCCCTCAATGA |
| | | CTCCAGATACTGCCTGAGCAGCCGCCTGAGGGTCTCGG |
| | | CCACCTTCTGGCAGAACCCCCGCAACCACTTCCGCTGTC |
| | | AAGTCCAGTTCTACGGGCTCTCGGAGAATGACGAGTGG |
| | | ACCCAGGATAGGGCCAAACCTGTCACCCAGATCGTCAG |
| | | CGCCGAGGCCTGGGGTAGAGCAGACTGTGGCTTCACCT |
| | | CCGAGTCTTACCAGCAAGGGGTCCTGTCTGCCACCATCC |
| | | TCTATGAGATCTTGCTAGGGAAGGCCACCTTGTATGCC |
| | | GTGCTGGTCAGTGCCCTCGTGCTGATGGCCATGGTCAA |
| | | GAGAAAGGATTCCAGAGGC |
| | | CGT GCC AAG CGA AGC GGA TCT GGC GCC ACG AAC |
| | | TTC TCT CTG TTA AAG CAA GCA GGA GAT GTT GAA |
| | | GAA AAC CCC GGG CCT |
| | | ATGTGGGGAGTTTTCCTTCTTTATGTTTCCATGAAGATG |
| | | GGAGGCACTACAGGACAAAACATTGACCAGCCCACTGA |
| | | GATGACAGCTACGGAAGGTGCCATTGTCCAGATCAACT |
| | | GCACGTACCAGACATCTGGGTTCAACGGGCTGTTCTGG |
| | | TACCAGCAACATGCTGGCGAAGCACCCACATTTCTGTCT |
| | | TACAATGTTCTGGATGGTTTGGAGGAGAAAGGTCGTTT |
| | | TTCTTCATTCCTTAGTCGGTCTAAAGGGTACAGTTACCTC |
| | | CTTTTGAAGGAGCTCCAGATGAAAGACTCTGCCTCTTAC |
| | | CTCTGTGCTGTGAAGGGTAGGAATTCCGGGTATGCACT |
| | | CAACTTCGGCAAAGGCACCTCGCTGTTGGTCACACCCCA |
| | | TATCCAGAACCCTGACCCTGCCGTGTACCAGCTGAGAG |
| | | ACTCTAAATCCAGTGACAAGTCTGTCTGCCTATTCACCG |
| | | ATTTTGATTCTCAAACAAATGTGTCACAAAGTAAGGATT |
| | | CTGATGTGTATATCACAGACAAATGCGTGCTAGACATG |
| | | AGGTCTATGGACTTCAAGAGCAACAGTGCTGTGGCCTG |
| | | GAGCAACAAATCTGACTTTGCATGTGCAAACGCCTTCAA |
| | | CAACAGCATTATTCCAGAAGACACCTTCTTCCCCAGCCC |
| | | AGAAAGTTCCTGTGATGTCAAGCTGGTCGAGAAAAGCT |
| | | TTGAAACAGATACGAACCTAAACTTTCAAAACCTGTCAG |
| | | TGATTGGGTTCCGAATCCTCCTCCTGAAAGTGGCCGGG |
| | | TTTAATCTGCTCATGACGCTGCGGCTGTGGTCCAGCTGA |
| SEQ ID NO. 62 | full TCR sequence-2 | ATGGGCACCAGCCTCCTCTGCTGGATGGCCCTGTGTCTC |
| | | CTGGGGGCAGATCACGCAGATACTGGAGTCTCCCAGGA |
| | | CCCCAGACACAAGATCACAAAGAGGGGACAGAATGTA |
| | | ACTTTCAGGTGTGATCCAATTTCTGAACACAACCGCCTT |
| | | TATTGGTACCGACAGACCCTGGGGCAGGGCCCAGAGTT |
| | | TCTGACTTACTTCCAGAATGAAGCTCAACTAGAAAAATC |
| | | AAGGCTGCTCAGTGATCGGTTCTCTGCAGAGAGGCCTA |
| | | AGGGATCTTTCTCCACCTTGGAGATCCAGCGCACAGAG |
| | | CAGGGGGACTCGGCCATGTATCTCTGTGCCAGCAGCTT |
| | | TTCCAGGGGGCCAGAGACCCAGTACTTCGGGCCAGGCA |
| | | CGCGGCTCCTGGTGCTCGAGGACCTGAAAAACGTGTTC |
| | | CCACCCGAGGTCGCTGTGTTTGAGCCATCAGAAGCAGA |
| | | GATCTCCCACACCCAAAAGGCCACACTGGTGTGCCTGG |
| | | CCACAGGCTTCTACCCCGACCACGTGGAGCTGAGCTGG |
| | | TGGGTGAATGGGAAGGAGGTGCACAGTGGGGTCTGCA |
| | | CAGACCCGCAGCCCCTCAAGGAGCAGCCCGCCCTCAAT |
| | | GACTCCAGATACTGCCTGAGCAGCCGCCTGAGGGTCTC |
| | | GGCCACCTTCTGGCAGAACCCCCGCAACCACTTCCGCTG |
| | | TCAAGTCCAGTTCTACGGGCTCTCGGAGAATGACGAGT |
| | | GGACCCAGGATAGGGCCAAACCTGTCACCCAGATCGTC |
| | | AGCGCCGAGGCCTGGGGTAGAGCAGACTGTGGCTTCA |
| | | CCTCCGAGTCTTACCAGCAAGGGGTCCTGTCTGCCACCA |
| | | TCCTCTATGAGATCTTGCTAGGGAAGGCCACCTTGTATG |
| | | CCGTGCTGGTCAGTGCCCTCGTGCTGATGGCCATGGTC |
| | | AAGAGAAAGGATTCCAGAGGC |
| | | CGT GCC AAG CGA AGC GGA TCT GGC GCC ACG AAC |
| | | TTC TCT CTG TTA AAG CAA GCA GGA GAT GTT GAA |
| | | GAA AAC CCC GGG CCT |
| | | ATGGCCATGCTCCTGGGGGCATCAGTGCTGATTCTGTG |
| | | GCTTCAGCCAGACTGGGTAAACAGTCAACAGAAGAATG |
| | | ATGACCAGCAAGTTAAGCAAAATTCACCATCCCTGAGC |
| | | GTCCAGGAAGGAAGAATTTCTATTCTGAACTGTGACTAT |
| | | ACTAACAGCATGTTTGATTATTTCCTATGGTACAAAAAA |
| | | TACCCTGCTGAAGGTCCTACATTCCTGATATCTATAAGT |
| | | TCCATTAAGGATAAAAATGAAGATGGAAGATTCACTGT |
| | | CTTCTTAAACAAAAGTGCCAAGCACCTCTCTCTGCACAT |
| | | TGTGCCCTCCCAGCCTGGAGACTCTGCAGTGTACTTCTG |
| | | TGCAGCAAGCGAGCGAGGCAGAGATGACAAGATCATC |
| | | TTTGGAAAAGGGACACGACTTCATATTCTCCCCAATATC |
| | | CAGAACCCTGACCCTGCCGTGTACCAGCTGAGAGACTC |
| | | TAAATCCAGTGACAAGTCTGTCTGCCTATTCACCGATTT |
| | | TGATTCTCAAACAAATGTGTCACAAAGTAAGGATTCTGA |
| | | TGTGTATATCACAGACAAATGCGTGCTAGACATGAGGT |
| | | CTATGGACTTCAAGAGCAACAGTGCTGTGGCCTGGAGC |
| | | AACAAATCTGACTTTGCATGTGCAAACGCCTTCAACAAC |
| | | AGCATTATTCCAGAAGACACCTTCTTCCCCAGCCCAGAA |
| | | AGTTCCTGTGATGTCAAGCTGGTCGAGAAAAGCTTTGA |
| | | AACAGATACGAACCTAAACTTTCAAAACCTGTCAGTGAT |
| | | TGGGTTCCGAATCCTCCTCCTGAAAGTGGCCGGGTTTAA |
| | | TCTGCTCATGACGCTGCGGCTGTGGTCCAGCTGA |
| SEQ ID NO. 63 | Full Alpha Chain | MWGVFLLYVSMKMGGTTGQNIDQPTEMTATEGAIVQI |
| | (including signal | NCTYQTSGFNGLFWYQQHAGEAPTFLSYNVLDGLEEKGR |
| | peptide)-1 | FSSFLSRSKGYSYLLLKELQMKDSASYLCAVKGRNSGYALN |
| | | FGKGTSLLVTPHIQNPDPAVYQLRDSKSSDKSVCLFTDFDS |
| | | QTNVSQSKDSDVYITDKCVLDMRSMDFKSNSAVAWSNK |
| | | SDFACANAFNNSIIPEDTFFPSPESSCDVKLVEKSFETDTNL |
| | | NFQNLSVIGFRILLLKVAGFNLLMTLRLWSS |
| SEQ ID NO. 64 | Full Alpha Chain | MAMLLGASVLILWLQPDWVNSQQKNDDQQVKQNSPSL |
| | (including signal | SVQEGRISILNCDYTNSMFDYFLWYKKYPAEGPTFLISISSIK |
| | peptide)-2 | DKNEDGRFTVFLNKSAKHLSLHIVPSQPGDSAVYFCAASER |
| | | GRDDKIIFGKGTRLHILPNIQNPDPAVYQLRDSKSSDKSVCL |
| | | FTDFDSQTNVSQSKDSDVYITDKCVLDMRSMDFKSNSAV |
| | | AWSNKSDFACANAFNNSIIPEDTFFPSPESSCDVKLVEKSF |
| | | ETDTNLNFQNLSVIGFRILLLKVAGFNLLMTLRLWSS |
| SEQ ID NO. 65 | Full Alpha Chain | ATGTGGGGAGTTTTCCTTCTTTATGTTTCCATGAAGATG |
| | (including signal | GGAGGCACTACAGGACAAAACATTGACCAGCCCACTGA |
| | peptide)-1 | GATGACAGCTACGGAAGGTGCCATTGTCCAGATCAACT |
| | | GCACGTACCAGACATCTGGGTTCAACGGGCTGTTCTGG |
| | | TACCAGCAACATGCTGGCGAAGCACCCACATTTCTGTCT |
| | | TACAATGTTCTGGATGGTTTGGAGGAGAAAGGTCGTTT |
| | | TTCTTCATTCCTTAGTCGGTCTAAAGGGTACAGTTACCTC |
| | | CTTTTGAAGGAGCTCCAGATGAAAGACTCTGCCTCTTAC |
| | | CTCTGTGCTGTGAAGGGTAGGAATTCCGGGTATGCACT |
| | | CAACTTCGGCAAAGGCACCTCGCTGTTGGTCACACCCCA |
| | | TATCCAGAACCCTGACCCTGCCGTGTACCAGCTGAGAG |
| | | ACTCTAAATCCAGTGACAAGTCTGTCTGCCTATTCACCG |
| | | ATTTTGATTCTCAAACAAATGTGTCACAAAGTAAGGATT |
| | | CTGATGTGTATATCACAGACAAATGCGTGCTAGACATG |
| | | AGGTCTATGGACTTCAAGAGCAACAGTGCTGTGGCCTG |
| | | GAGCAACAAATCTGACTTTGCATGTGCAAACGCCTTCAA |
| | | CAACAGCATTATTCCAGAAGACACCTTCTTCCCCAGCCC |
| | | AGAAAGTTCCTGTGATGTCAAGCTGGTCGAGAAAAGCT |
| | | TTGAAACAGATACGAACCTAAACTTTCAAAACCTGTCAG |
| | | TGATTGGGTTCCGAATCCTCCTCCTGAAAGTGGCCGGG |
| | | TTTAATCTGCTCATGACGCTGCGGCTGTGGTCCAGCTGA |
| SEQ ID NO. 66 | Full Alpha Chain | ATGGCCATGCTCCTGGGGGCATCAGTGCTGATTCTGTG |
| | (including signal | GCTTCAGCCAGACTGGGTAAACAGTCAACAGAAGAATG |
| | peptide)-2 | ATGACCAGCAAGTTAAGCAAAATTCACCATCCCTGAGC |
| | | GTCCAGGAAGGAAGAATTTCTATTCTGAACTGTGACTAT |
| | | ACTAACAGCATGTTTGATTATTTCCTATGGTACAAAAAA |
| | | TACCCTGCTGAAGGTCCTACATTCCTGATATCTATAAGT |
| | | TCCATTAAGGATAAAAATGAAGATGGAAGATTCACTGT |
| | | CTTCTTAAACAAAAGTGCCAAGCACCTCTCTCTGCACAT |
| | | TGTGCCCTCCCAGCCTGGAGACTCTGCAGTGTACTTCTG |
| | | TGCAGCAAGCGAGCGAGGCAGAGATGACAAGATCATC |
| | | TTTGGAAAAGGGACACGACTTCATATTCTCCCCAATATC |
| | | CAGAACCCTGACCCTGCCGTGTACCAGCTGAGAGACTC |
| | | TAAATCCAGTGACAAGTCTGTCTGCCTATTCACCGATTT |
| | | TGATTCTCAAACAAATGTGTCACAAAGTAAGGATTCTGA |
| | | TGTGTATATCACAGACAAATGCGTGCTAGACATGAGGT |
| | | CTATGGACTTCAAGAGCAACAGTGCTGTGGCCTGGAGC |
| | | AACAAATCTGACTTTGCATGTGCAAACGCCTTCAACAAC |
| | | AGCATTATTCCAGAAGACACCTTCTTCCCCAGCCCAGAA |
| | | AGTTCCTGTGATGTCAAGCTGGTCGAGAAAAGCTTTGA |
| | | AACAGATACGAACCTAAACTTTCAAAACCTGTCAGTGAT |
| | | TGGGTTCCGAATCCTCCTCCTGAAAGTGGCCGGGTTTAA |
| | | TCTGCTCATGACGCTGCGGCTGTGGTCCAGCTGA |
| SEQ ID NO. 67 | Full Beta Chain | MGFRLLCCVAFCLLGAGPVDSGVTQTPKHLITATGQRVTL |
| | (including signal | RCSPRSGDLSVYWYQQSLDQGLQFLIQYYNGEERAKGNIL |
| | peptide)-1 | ERFSAQQFPDLHSELNLSSLELGDSALYFCASTIQGAAQET |
| | | QYFGPGTRLLVLEDLKNVFPPEVAVFEPSEAEISHTQKATL |
| | | VCLATGFYPDHVELSWWVNGKEVHSGVCTDPQPLKEQP |
| | | ALNDSRYCLSSRLRVSATFWQNPRNHFRCQVQFYGLSEN |
| | | DEWTQDRAKPVTQIVSAEAWGRADCGFTSESYQQGVLS |
| | | ATILYEILLGKATLYAVLVSALVLMAMVKRKDSRG |
| SEQ ID NO. 68 | Full Beta Chain | MGTSLLCWMALCLLGADHADTGVSQDPRHKITKRGQNV |
| | (including signal | TFRCDPISEHNRLYWYRQTLGQGPEFLTYFQNEAQLEKSR |
| | peptide)-2 | LLSDRFSAERPKGSFSTLEIQRTEQGDSAMYLCASSFSRGP |
| | | ETQYFGPGTRLLVLEDLKNVFPPEVAVFEPSEAEISHTQKA |
| | | TLVCLATGFYPDHVELSWWVNGKEVHSGVCTDPQPLKEQ |
| | | PALNDSRYCLSSRLRVSATFWQNPRNHFRCQVQFYGLSE |
| | | NDEWTQDRAKPVTQIVSAEAWGRADCGFTSESYQQGVL |
| | | SATILYEILLGKATLYAVLVSALVLMAMVKRKDSRG |
| SEQ ID NO. 69 | Full Beta chain | ATGGGCTTCAGGCTCCTCTGCTGTGTGGCCTTTTGTCTC |
| | (including signal | CTGGGAGCAGGCCCAGTGGATTCTGGAGTCACACAAAC |
| | peptide)-1 | CCCAAAGCACCTGATCACAGCAACTGGACAGCGAGTGA |
| | | CGCTGAGATGCTCCCCTAGGTCTGGAGACCTCTCTGTGT |
| | | ACTGGTACCAACAGAGCCTGGACCAGGGCCTCCAGTTC |
| | | CTCATTCAGTATTATAATGGAGAAGAGAGAGCAAAAGG |
| | | AAACATTCTTGAACGATTCTCCGCACAACAGTTCCCTGA |
| | | CTTGCACTCTGAACTAAACCTGAGCTCTCTGGAGCTGGG |
| | | GGACTCAGCTTTGTATTTCTGTGCCAGCACTATACAGGG |
| | | GGCGGCTCAAGAGACCCAGTACTTCGGGCCAGGCACG |
| | | CGGCTCCTGGTGCTCGAGGACCTGAAAAACGTGTTCCC |
| | | ACCCGAGGTCGCTGTGTTTGAGCCATCAGAAGCAGAGA |
| | | TCTCCCACACCCAAAAGGCCACACTGGTGTGCCTGGCC |
| | | ACAGGCTTCTACCCCGACCACGTGGAGCTGAGCTGGTG |
| | | GGTGAATGGGAAGGAGGTGCACAGTGGGGTCTGCACA |
| | | GACCCGCAGCCCCTCAAGGAGCAGCCCGCCCTCAATGA |
| | | CTCCAGATACTGCCTGAGCAGCCGCCTGAGGGTCTCGG |
| | | CCACCTTCTGGCAGAACCCCCGCAACCACTTCCGCTGTC |
| | | AAGTCCAGTTCTACGGGCTCTCGGAGAATGACGAGTGG |
| | | ACCCAGGATAGGGCCAAACCTGTCACCCAGATCGTCAG |
| | | CGCCGAGGCCTGGGGTAGAGCAGACTGTGGCTTCACCT |
| | | CCGAGTCTTACCAGCAAGGGGTCCTGTCTGCCACCATCC |
| | | TCTATGAGATCTTGCTAGGGAAGGCCACCTTGTATGCC |
| | | GTGCTGGTCAGTGCCCTCGTGCTGATGGCCATGGTCAA |
| | | GAGAAAGGATTCCAGAGGC |
| SEQ ID NO. 70 | Full Beta chain | ATGGGCACCAGCCTCCTCTGCTGGATGGCCCTGTGTCTC |
| | (including signal | CTGGGGGCAGATCACGCAGATACTGGAGTCTCCCAGGA |
| | peptide)-2 | CCCCAGACACAAGATCACAAAGAGGGGACAGAATGTA |
| | | ACTTTCAGGTGTGATCCAATTTCTGAACACAACCGCCTT |
| | | TATTGGTACCGACAGACCCTGGGGCAGGGCCCAGAGTT |
| | | TCTGACTTACTTCCAGAATGAAGCTCAACTAGAAAAATC |
| | | AAGGCTGCTCAGTGATCGGTTCTCTGCAGAGAGGCCTA |
| | | AGGGATCTTTCTCCACCTTGGAGATCCAGCGCACAGAG |
| | | CAGGGGGACTCGGCCATGTATCTCTGTGCCAGCAGCTT |
| | | TTCCAGGGGGCCAGAGACCCAGTACTTCGGGCCAGGCA |
| | | CGCGGCTCCTGGTGCTCGAGGACCTGAAAAACGTGTTC |
| | | CCACCCGAGGTCGCTGTGTTTGAGCCATCAGAAGCAGA |
| | | GATCTCCCACACCCAAAAGGCCACACTGGTGTGCCTGG |
| | | CCACAGGCTTCTACCCCGACCACGTGGAGCTGAGCTGG |
| | | TGGGTGAATGGGAAGGAGGTGCACAGTGGGGTCTGCA |
| | | CAGACCCGCAGCCCCTCAAGGAGCAGCCCGCCCTCAAT |
| | | GACTCCAGATACTGCCTGAGCAGCCGCCTGAGGGTCTC |
| | | GGCCACCTTCTGGCAGAACCCCCGCAACCACTTCCGCTG |
| | | TCAAGTCCAGTTCTACGGGCTCTCGGAGAATGACGAGT |
| | | GGACCCAGGATAGGGCCAAACCTGTCACCCAGATCGTC |
| | | AGCGCCGAGGCCTGGGGTAGAGCAGACTGTGGCTTCA |
| | | CCTCCGAGTCTTACCAGCAAGGGGTCCTGTCTGCCACCA |
| | | TCCTCTATGAGATCTTGCTAGGGAAGGCCACCTTGTATG |
| | | CCGTGCTGGTCAGTGCCCTCGTGCTGATGGCCATGGTC |
| | | AAGAGAAAGGATTCCAGAGGC |
| SEQ ID NO. 71 | Alpha Chain C/V (not | CAAAACATTGACCAGCCCACTGAGATGACAGCTACGGA |
| | including signal | AGGTGCCATTGTCCAGATCAACTGCACGTACCAGACAT |
| | peptide)-1 | CTGGGTTCAACGGGCTGTTCTGGTACCAGCAACATGCT |
| | | GGCGAAGCACCCACATTTCTGTCTTACAATGTTCTGGAT |
| | | GGTTTGGAGGAGAAAGGTCGTTTTTCTTCATTCCTTAGT |
| | | CGGTCTAAAGGGTACAGTTACCTCCTTTTGAAGGAGCTC |
| | | CAGATGAAAGACTCTGCCTCTTACCTCTGTGCTGTGAAG |
| | | GGTAGGAATTCCGGGTATGCACTCAACTTCGGCAAAGG |
| | | CACCTCGCTGTTGGTCACACCCCATATCCAGAACCCTGA |
| | | CCCTGCCGTGTACCAGCTGAGAGACTCTAAATCCAGTG |
| | | ACAAGTCTGTCTGCCTATTCACCGATTTTGATTCTCAAAC |
| | | AAATGTGTCACAAAGTAAGGATTCTGATGTGTATATCAC |
| | | AGACAAATGCGTGCTAGACATGAGGTCTATGGACTTCA |
| | | AGAGCAACAGTGCTGTGGCCTGGAGCAACAAATCTGAC |
| | | TTTGCATGTGCAAACGCCTTCAACAACAGCATTATTCCA |
| | | GAAGACACCTTCTTCCCCAGCCCAGAAAGTTCCTGTGAT |
| | | GTCAAGCTGGTCGAGAAAAGCTTTGAAACAGATACGAA |
| | | CCTAAACTTTCAAAACCTGTCAGTGATTGGGTTCCGAAT |
| | | CCTCCTCCTGAAAGTGGCCGGGTTTAATCTGCTCATGAC |
| | | GCTGCGGCTGTGGTCCAGCTGA |
| SEQ ID NO. 72 | Alpha Chain C/V (not | CAACAGAAGAATGATGACCAGCAAGTTAAGCAAAATTC |
| | including signal | ACCATCCCTGAGCGTCCAGGAAGGAAGAATTTCTATTCT |
| | peptide)-2 | GAACTGTGACTATACTAACAGCATGTTTGATTATTTCCT |
| | | ATGGTACAAAAAATACCCTGCTGAAGGTCCTACATTCCT |
| | | GATATCTATAAGTTCCATTAAGGATAAAAATGAAGATG |
| | | GAAGATTCACTGTCTTCTTAAACAAAAGTGCCAAGCACC |
| | | TCTCTCTGCACATTGTGCCCTCCCAGCCTGGAGACTCTG |
| | | CAGTGTACTTCTGTGCAGCAAGCGAGCGAGGCAGAGAT |
| | | GACAAGATCATCTTTGGAAAAGGGACACGACTTCATAT |
| | | TCTCCCCAATATCCAGAACCCTGACCCTGCCGTGTACCA |
| | | GCTGAGAGACTCTAAATCCAGTGACAAGTCTGTCTGCCT |
| | | ATTCACCGATTTTGATTCTCAAACAAATGTGTCACAAAG |
| | | TAAGGATTCTGATGTGTATATCACAGACAAATGCGTGCT |
| | | AGACATGAGGTCTATGGACTTCAAGAGCAACAGTGCTG |
| | | TGGCCTGGAGCAACAAATCTGACTTTGCATGTGCAAAC |
| | | GCCTTCAACAACAGCATTATTCCAGAAGACACCTTCTTC |
| | | CCCAGCCCAGAAAGTTCCTGTGATGTCAAGCTGGTCGA |
| | | GAAAAGCTTTGAAACAGATACGAACCTAAACTTTCAAA |
| | | ACCTGTCAGTGATTGGGTTCCGAATCCTCCTCCTGAAAG |
| | | TGGCCGGGTTTAATCTGCTCATGACGCTGCGGCTGTGG |
| | | TCCAGCTGA |
| SEQ ID NO. 73 | Beta Chain C/V (not | GGAGTCACACAAACCCCAAAGCACCTGATCACAGCAAC |
| | including signal | TGGACAGCGAGTGACGCTGAGATGCTCCCCTAGGTCTG |
| | peptide)-1 | GAGACCTCTCTGTGTACTGGTACCAACAGAGCCTGGAC |
| | | CAGGGCCTCCAGTTCCTCATTCAGTATTATAATGGAGAA |
| | | GAGAGAGCAAAAGGAAACATTCTTGAACGATTCTCCGC |
| | | ACAACAGTTCCCTGACTTGCACTCTGAACTAAACCTGAG |
| | | CTCTCTGGAGCTGGGGGACTCAGCTTTGTATTTCTGTGC |
| | | CAGCACTATACAGGGGGGGGCTCAAGAGACCCAGTACT |
| | | TCGGGCCAGGCACGCGGCTCCTGGTGCTCGAGGACCTG |
| | | AAAAACGTGTTCCCACCCGAGGTCGCTGTGTTTGAGCC |
| | | ATCAGAAGCAGAGATCTCCCACACCCAAAAGGCCACAC |
| | | TGGTGTGCCTGGCCACAGGCTTCTACCCCGACCACGTG |
| | | GAGCTGAGCTGGTGGGTGAATGGGAAGGAGGTGCACA |
| | | GTGGGGTCTGCACAGACCCGCAGCCCCTCAAGGAGCA |
| | | GCCCGCCCTCAATGACTCCAGATACTGCCTGAGCAGCC |
| | | GCCTGAGGGTCTCGGCCACCTTCTGGCAGAACCCCCGC |
| | | AACCACTTCCGCTGTCAAGTCCAGTTCTACGGGCTCTCG |
| | | GAGAATGACGAGTGGACCCAGGATAGGGCCAAACCTG |
| | | TCACCCAGATCGTCAGCGCCGAGGCCTGGGGTAGAGCA |
| | | GACTGTGGCTTCACCTCCGAGTCTTACCAGCAAGGGGT |
| | | CCTGTCTGCCACCATCCTCTATGAGATCTTGCTAGGGAA |
| | | GGCCACCTTGTATGCCGTGCTGGTCAGTGCCCTCGTGCT |
| | | GATGGCCATGGTCAAGAGAAAGGATTCCAGAGGC |
| SEQ ID NO. 74 | Beta Chain C/V (not | GGAGTCTCCCAGGACCCCAGACACAAGATCACAAAGAG |
| | including signal | GGGACAGAATGTAACTTTCAGGTGTGATCCAATTTCTG |
| | peptide)-2 | AACACAACCGCCTTTATTGGTACCGACAGACCCTGGGG |
| | | CAGGGCCCAGAGTTTCTGACTTACTTCCAGAATGAAGCT |
| | | CAACTAGAAAAATCAAGGCTGCTCAGTGATCGGTTCTCT |
| | | GCAGAGAGGCCTAAGGGATCTTTCTCCACCTTGGAGAT |
| | | CCAGCGCACAGAGCAGGGGGACTCGGCCATGTATCTCT |
| | | GTGCCAGCAGCTTTTCCAGGGGGCCAGAGACCCAGTAC |
| | | TTCGGGCCAGGCACGCGGCTCCTGGTGCTCGAGGACCT |
| | | GAAAAACGTGTTCCCACCCGAGGTCGCTGTGTTTGAGC |
| | | CATCAGAAGCAGAGATCTCCCACACCCAAAAGGCCACA |
| | | CTGGTGTGCCTGGCCACAGGCTTCTACCCCGACCACGT |
| | | GGAGCTGAGCTGGTGGGTGAATGGGAAGGAGGTGCAC |
| | | AGTGGGGTCTGCACAGACCCGCAGCCCCTCAAGGAGCA |
| | | GCCCGCCCTCAATGACTCCAGATACTGCCTGAGCAGCC |
| | | GCCTGAGGGTCTCGGCCACCTTCTGGCAGAACCCCCGC |
| | | AACCACTTCCGCTGTCAAGTCCAGTTCTACGGGCTCTCG |
| | | GAGAATGACGAGTGGACCCAGGATAGGGCCAAACCTG |
| | | TCACCCAGATCGTCAGCGCCGAGGCCTGGGGTAGAGCA |
| | | GACTGTGGCTTCACCTCCGAGTCTTACCAGCAAGGGGT |
| | | CCTGTCTGCCACCATCCTCTATGAGATCTTGCTAGGGAA |
| | | GGCCACCTTGTATGCCGTGCTGGTCAGTGCCCTCGTGCT |
| | | GATGGCCATGGTCAAGAGAAAGGATTCCAGAGGC |

What is claimed is:

1. A recombinant T-cell Receptor (TCR) comprising: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 68, and (iii) a CDR3β of SEQ ID NO: 17, 18, 35, 36, 67, or 68, wherein the recombinant TCR binds an epitope of a Kirsten Rat Sarcoma Viral Oncogene Homolog (KRAS) protein comprising a G12V mutation presented on a human leucocyte antigen-A (HLA-A)*03:01, wherein the epitope comprises the amino acid sequence of VVGAVGVGK (SEQ ID NO: 1).

2. The recombinant T-cell Receptor (TCR) of claim 1, wherein the TCR alpha chain comprises the CDR1α, the CDR2α, and the CDR3α of SEQ ID NO: 15, 31 or 63, and the TCR beta chain comprises the CDR1β, the CDR2β, and the CDR3β of SEQ ID NO: 17, 35, or 67.

3. The recombinant T-cell Receptor (TCR) of claim 1, wherein the TCR alpha chain comprises the CDR1α, the CDR2α, and the CDR3α of SEQ ID NO: 16, 32, or 64, and the TCR beta chain comprises the CDR1β, the CDR2β, and the CDR3β of SEQ ID NO: 18, 36, or 68.

4. The recombinant T cell receptor (TCR) of any one of claims 1-3, wherein
(i) the CDR1α comprises the sequence of SEQ ID NO: 3, or 4,
(ii) the CDR2α comprises the sequence of SEQ ID NO: 5, or 6,
(iii) the CDR3α comprises the sequence of SEQ ID NO: 7, or 8,
(iv) the CDR1β comprises the sequence of SEQ ID NO: 9, or 10,
(v) the CDR2β comprises the sequence of SEQ ID NO: 11, or 12, and
(vi) the CDR3β comprises the sequence of SEQ ID NO: 13, or 14.

5. The recombinant T cell receptor (TCR) of claim 4, wherein
(i) the CDR1α comprises the sequence of SEQ ID NO: 3,
(ii) the CDR2α comprises the sequence of SEQ ID NO: 5,
(iii) the CDR3α comprises the sequence of SEQ ID NO: 7,
(iv) the CDR1β comprises the sequence of SEQ ID NO: 9,
(v) the CDR2β comprises the sequence of SEQ ID NO: 11, and
(vi) the CDR3β comprises the sequence of SEQ ID NO: 13.

6. The recombinant T cell receptor (TCR) of claim 4, wherein
(i) the CDR1α comprises the sequence of SEQ ID NO: 4,
(ii) the CDR2α comprises the sequence of SEQ ID NO: 6,
(iii) the CDR3α comprises the sequence of SEQ ID NO: 8,
(iv) the CDR1β comprises the sequence of SEQ ID NO: 10,
(v) the CDR2β comprises the sequence of SEQ ID NO: 12, and
(vi) the CDR3β comprises the sequence of SEQ ID NO: 14.

7. The recombinant T cell receptor (TCR) of any one of claims 1-6, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68.

8. The recombinant T cell receptor (TCR) of claim 7, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31, or 63; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 35, or 67.

9. The recombinant T cell receptor (TCR) of claim 7, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 18, 36, or 68.

10. The recombinant T cell receptor (TCR) of any one of claims 1-9, wherein the TCR alpha chain comprises an amino acid sequence of SEQ ID NO: 15, 16, 31, 32, 63, or 64, and the TCR beta chain comprises an amino acid sequence of SEQ ID NO: 17, 18, 35, 36, 67, or 68.

11. The recombinant T cell receptor (TCR) of claim 10, wherein the TCR alpha chain comprises an amino acid sequence of SEQ ID NO: 15, 31, or 63, and the TCR beta chain comprises an amino acid sequence of SEQ ID NO: 17, 35, or 67.

12. The recombinant T cell receptor (TCR) of any one of claim 10, wherein the TCR alpha chain comprises an amino acid sequence of SEQ ID NO: 16, 32, or 64, and the TCR beta chain comprises an amino acid sequence of SEQ ID NO: 18, 36, or 68.

13. The recombinant TCR of any one of claims 1-12, wherein the TCR is humanized.

14. The recombinant TCR of any one of claims 1-12, wherein the TCR is chimeric.

15. A polynucleotide encoding the recombinant TCR of any one of claims 1-14.

16. A polynucleotide encoding the TCR alpha chain of the recombinant TCR of any one of claims 1-14.

17. A polynucleotide encoding the TCR beta chain of the recombinant TCR of any one of claims 1-14.

18. An expression vector comprising the polynucleotide of any one of claims 15-17.

19. A host cell comprising the T cell receptor (TCR) of any one of claims 1-14, the polynucleotide of any one of claims 15-17, and/or the expression vector of claim 18.

20. The host cell of claim 19, wherein the host cell is a T cell.

21. The host cell of any one of claims 19 or 20, wherein the host cell is a CD4+ cell.

22. The host cell of any one of claims 19-21, wherein the host cell is a CD8+ cell.

23. A T cell comprising the TCR of any one of claims 1-14, the polynucleotide of any one of claims 15-17, or the expression vector of claim 18.

24. The T cell of claim 23, wherein the T cell comprises a CD4+ cell.

25. The T cell of any one of claims 23 or 24, wherein the T cell comprises a CD8+ cell.

26. A modified T cell comprising the recombinant T cell receptor (TCR) of any one of claims 1-14 or the polynucleotide of any one of claims 15-17, or the expression vector of claim 18.

27. The modified T cell of claim 26, wherein the T cell receptor (TCR) is expressed on the surface of the modified T cell.

28. The modified T cell of any one of claims 26 or 27, wherein the modified T cell is capable of recognition of a cancer cell expressing the Kirsten Rat Sarcoma Viral Oncogene Homolog (KRAS) protein comprising the G12V mutation which comprises an epitope presented on the human leucocyte antigen-A (HLA-A)*03:01, wherein the epitope comprises the amino acid sequence of VVGAVGVGK (SEQ ID NO: 1).

29. The modified T cell of claim 28, wherein the recognition of the cancer cell comprises a human leucocyte antigen-A (HLA-A)*03:01-restricted binding of the TCR to the epitope of the Kirsten Rat Sarcoma Viral Oncogene Homolog (KRAS) protein comprising the G12V mutation expressed by the cancer cell.

30. The modified T cell of any one of claims 26-29, wherein the recombinant TCR is humanized.

31. The modified T cell of any one of claims 26-29, wherein the recombinant TCR is chimeric.

32. A Bi-specific T-cell engager (BiTE) comprising the recombinant TCR of any one of claims 1-14.

33. A modified T cell comprising a T cell receptor (TCR) comprising: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2β of SEQ ID NO: 17, 18, 35, 36, 67, or 87, and (iii) a CDR3β of SEQ ID NO: 17, 18, 35, 36, 67, or 68, wherein the TCR binds an epitope of a Kirsten Rat Sarcoma Viral Oncogene Homolog (KRAS) protein comprising a G12V mutation presented on a human leucocyte antigen-A (HLA-A)*03:01, wherein the epitope comprises the amino acid sequence of VVGAVGVGK (SEQ ID NO: 1).

34. The modified T cell of claim 33, wherein the TCR alpha chain comprises the CDR1α, the CDR2α, and the CDR3α of SEQ ID NO: 15, 31 or 63, and wherein the TCR beta chain comprises the CDR1β, the CDR2β, and the CDR3β of SEQ ID NO: 17, 35, or 67.

35. The modified T cell of claim 33, wherein the TCR alpha chain comprises the CDR1α, the CDR2α, and the CDR3α of SEQ ID NO: 16, 32, or 64, and wherein the TCR beta chain comprises the CDR1β, the CDR2β, and the CDR3β of SEQ ID NO: 18, 36, or 68.

36. The modified T cell of any one of claims 33-35, wherein the TCR is expressed on the surface of the modified T cell.

37. The modified T cell of any one of claims 33-36, wherein the modified T cell is capable of recognition of a cancer cell expressing the Kirsten Rat Sarcoma Viral Oncogene Homolog (KRAS) protein comprising the G12V mutation which comprises the epitope presented on the human leucocyte antigen-A (HLA-A)*03:01.

38. The modified T cell of claim 37, wherein the recognition of the cancer cell comprises a human leucocyte antigen-A (HLA-A)*03:01-restricted binding of the TCR to the epitope of the Kirsten Rat Sarcoma Viral Oncogene Homolog (KRAS) protein comprising the G12V mutation expressed by the cancer cell.

39. The modified T cell of any one of claims 33-38, wherein the TCR is humanized.

40. The modified T cell of any one of claims 33-38, wherein the TCR is chimeric.

41. A bi-specific T-cell engager (BiTE) comprising a T cell receptor (TCR) comprising: (a) a TCR alpha chain comprising (i) a variable TCR alpha chain complementary determining region 1 (CDR1α) of SEQ ID NO: 15, 16, 31, 32, 63, or 64, (ii) a CDR2α of SEQ ID NO: 15, 16, 31, 32, 63, or 64, and (iii) a CDR3α of SEQ ID NO: 15, 16, 31, 32, 63, or 64; and (b) a TCR beta chain comprising (i) a variable TCR beta chain complementary determining region 1 (CDR1β) of SEQ ID NO: 17, 18, 35, 36, 67, or 68, (ii) a CDR2B of SEQ ID NO: 17, 18, 35, 36, 67, or 68, and (iii) a CDR3β of SEQ ID NO: 17, 18, 35, 36, 67, or 68, wherein the TCR binds an epitope of a Kirsten Rat Sarcoma Viral Oncogene Homolog (KRAS) protein comprising a G12V mutation presented on a human leucocyte antigen-A (HLA-A)*03:01, wherein the epitope comprises the amino acid sequence of VVGAVGVGK (SEQ ID NO: 1).

42. The bi-specific T-cell engager (BiTE) of claim 41, wherein the TCR alpha chain comprises the CDR1α, the CDR2α, and the CDR3α of SEQ ID NO: 15, 31 or 63, and wherein the TCR beta chain comprises the CDR1β, the CDR2β, and the CDR3β of SEQ ID NO: 17, 35, or 67.

43. The bi-specific T-cell engager (BiTE) of claim 41, wherein the TCR alpha chain comprises the CDR1α, the CDR2α, and the CDR3α of SEQ ID NO: 16, 32, or 64, and wherein the TCR beta chain comprises the CDR1β, the CDR2β, and the CDR3β of SEQ ID NO: 18, 36, or 68.

44. The modified T cell of any one of claims 33-40 or the BiTE of any one of claims 41-43, wherein
(i) the CDR1α comprises the sequence of SEQ ID NO: 3, or 4,
(ii) the CDR2α comprises the sequence of SEQ ID NO: 5, or 6,
(iii) the CDR3α comprises the sequence of SEQ ID NO: 7, or 8,
(iv) the CDR1β comprises the sequence of SEQ ID NO: 9, or 10,
(v) the CDR2β comprises the sequence of SEQ ID NO: 11, or 12, and
(vi) the CDR3β comprises the sequence of SEQ ID NO: 13, or 14.

45. The modified T cell or the BiTE of claim 44, wherein
(i) the CDR1α comprises the sequence of SEQ ID NO: 3,
(ii) the CDR2α comprises the sequence of SEQ ID NO: 5,
(iii) the CDR3α comprises the sequence of SEQ ID NO: 7,
(iv) the CDR1β comprises the sequence of SEQ ID NO: 9,
(v) the CDR2β comprises the sequence of SEQ ID NO: 11, and
(vi) the CDR3β comprises the sequence of SEQ ID NO: 13.

46. The modified T cell or the BiTE of claim 44, wherein
(i) the CDR1α comprises the sequence of SEQ ID NO: 4,
(ii) the CDR2α comprises the sequence of SEQ ID NO: 6,
(iii) the CDR3α comprises the sequence of SEQ ID NO: 8,
(iv) the CDR1β comprises the sequence of SEQ ID NO: 10,
(v) the CDR2β comprises the sequence of SEQ ID NO: 12, and
(vi) the CDR3β comprises the sequence of SEQ ID NO: 14.

47. The modified T cell or the BiTE of any one of claims 33-46, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 16, 31, 32, 63, or 64; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 18, 35, 36, 67, or 68.

48. The modified T cell or the BiTE of claim 47, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 15, 31, or 63; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 17, 35, or 67.

49. The modified T cell or the BiTE of claim 47, wherein the TCR alpha chain comprises an amino acid sequence having at least 85%, at least 90%, at least 91% at least 92%, at least 93%, at least 94%, at least 95%, at least 96%, at least 97%, at least 98%, or at least 99% sequence identity to SEQ ID NO: 16, 32, or 64; and the TCR beta chain comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 18, 36, or 68.

50. The modified T cell or the BiTE of any one of claims 33-49, wherein the TCR alpha chain comprises an amino acid sequence of SEQ ID NO: 15, 16, 31, 32, 63, or 64, and the TCR beta chain comprises an amino acid sequence of SEQ ID NO: 17, 18, 35, 36, 67, or 68.

51. The modified T cell or the BiTE of claim 50, wherein the TCR alpha chain comprises an amino acid sequence of SEQ ID NO: 15, 31, or 63, and the TCR beta chain comprises an amino acid sequence of SEQ ID NO: 17, 35, or 67.

52. The modified T cell or the BiTE of claim 50, wherein the TCR alpha chain comprises an amino acid sequence of SEQ ID NO: 16, 32, or 64, and the TCR beta chain comprises an amino acid sequence of SEQ ID NO: 18, 36, or 68.

53. A method for prophylaxis and/or therapy of a subject diagnosed with, suspected of having or at risk for developing or recurrence of a cancer, comprising administering to the subject the recombinant TCR of any one of claims 1-14, the polynucleotide of any one of claims 15-17, the vector of claim 18, the host cell of any one of claims 19-22, the T cell of any one of claims 23-25, the modified T cell of any one of claims 26-31, 33-40 and 44-52, or the BiTE of any one of claims 32 and 41-52.

54. The method of claim 53, wherein the cancer comprises cancer cells expressing a Kirsten Rat Sarcoma Viral Oncogene Homolog (KRAS) protein comprising a G12V mutation which comprises the epitope presented on a human leucocyte antigen-A (HLA-A)*03:01.

55. The method of any one of claims 53 or 54, wherein the cancer is selected from the group consisting of: a lung cancer, a colorectal cancer, a pancreatic cancer, an appendiceal cancer, a small bowel adenocarcinoma, an hepatobiliary cancer, a gynecological malignancy, a hematopoietic cancer, a breast cancer, a bladder cancer, a prostate cancer, a skin cancer, or any combination thereof.

56. The method of any one of claims 53-55, wherein the cancer is selected from the group consisting of: a lung cancer, a non-small cell lung cancer (NSCLC), a colorectal cancer, a pancreatic cancer, or any combination thereof.

57. The method of any one of claims 53-56, wherein the cancer is a non-small cell lung cancer (NSCLC).

58. The method of any one of claims 53-57, comprising removing a T cell from the subject prior to the administering, and modifying the T cell by introducing into the T cell the recombinant TCR of any one of claims 1-14, the polynucleotide of any one of claims 15-17, or the vector of claim 18.

59. The method of any one of claims 53-58, wherein the subject is a human.
